(19) United States
(12) Patent Application Publication
(10) Pub. No.: US 2023/0313301 A1
(43) Pub. Date: Oct. 5, 2023

(54) METHODS OF IDENTIFYING AUTISM SPECTRUM DISORDER

(71) Applicant: WASHINGTON STATE UNIVERSITY, Pullman, WA (US)

(72) Inventor: Michael SKINNER, Pullman, WA (US)

(21) Appl. No.: 18/006,200

(22) PCT Filed: Aug. 18, 2021
(86) PCT No.: PCT/US2021/046422

§ 371 Date: Jan. 20, 2023

Related U.S. Application Data
(60) Provisional application No. 63/055,199, filed on Jul. 22, 2020.

(51) Int. Cl. C12Q 1/6883; C12Q 1/6869; C12N 15/10

(52) U.S. Cl. CPC C12Q 1/6883; C12Q 1/6869; C12N 15/10; C12Q 2600/112

(57) ABSTRACT

Described herein are methods of identifying one or more epigenetic modifications in a nucleic acid sequence of a sample of a subject's father to identify a risk of autism spectrum disorder (ASD), to diagnose ASD early in a subject based at least in part of the epigenetic modification identified in the nucleic acid sequence of the sample of the subject's father. Also disclosed herein are methods of treating a subject with autism or ASD.

### CROSS REFERENCE TO RELATED APPLICATIONS

This application claims the benefit of U.S. Provisional Application Ser. No. 63/055,199, filed Jul. 22, 2020, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Autism spectrum disorder (ASD) is a complex neurological disorder involving deficits in communication, social behaviors and stereotypic movements. The prevalence of ASD in 1975 was reported as 1 in 5000 and then in 2009 as 1 in 110. The American Centers for Disease Control and Prevention reported a 1 in 88 prevalence in 2012 and then a 1 in 68 in 2014. Although improved diagnosis and current awareness have played a role in this increase, particularly in the first couple decades (1975-2000), the increase in the last two decades is thought to be due to environmental and molecular factors. This is supported by twin studies and numerous environmental studies. Genetic studies using genome-wide association studies (GWAS) have identified multiple genetic mutations, but they are correlated with a small percentage of the autism patients. Combining genetic mutations and altered epigenetics appear to improve this association. Many specific toxicants and factors have been suggested to be involved, but generally more extensive analysis is required. Environmental factors are now believed to be involved in the etiology of autism, however, the specific environmental factors, molecular processes, and etiology of autism remain to be fully elucidated.

### SUMMARY

Disclosed herein are methods, comprising: obtaining a sperm sample from a human male subject; isolating deoxyribonucleic acid (DNA) from the sample; determining a methylation level of a differential DNA methylation region (DMR) comprised in the isolated DNA; and comparing the methylation level of the DMR to a reference level of a corresponding reference DMR. In some embodiments, the comparing can comprise comparing employing a computer comprising a computer processor and computer readable memory comprising computer readable instructions contained thereon In some embodiments, the determining can comprise a methylated DNA immunoprecipitation (MeDIP), a sequencing, a bisulfite treatment, a bisulfite conversion, a deamination of an unmethylated cytosine base, employing an array, or any combination of these. In some embodiments, about 90 to about 1000 distinct DMRs can be detected and compared; and the about 90 to about 1000 distinct DMRs can be selected from the DMRs in Table 3. In some embodiments, about 200 to about 1000 distinct DMRs, about 300 to about 1000 distinct DMRs, about 400 to about 1000 distinct DMRs, about 500 to about 1000 distinct DMRs, about 600 to about 1000 distinct DMRs, about 700 to about 1000 distinct DMRs, about 800 to about 1000, or about 900 to about 1000 distinct DMRs can be detected. In some embodiments, the method can comprise sequencing, and the sequencing can comprise sequencing by synthesis, ion semiconductor sequencing, single molecule real time sequencing, nanopore sequencing, next-generation sequencing, or any combination thereof. In some embodiments, the detected DMRs can comprise DMRs from at least about: 1, 2, 3, 4, 5, 6, 7, 8, 9, 10, 11, 12, 13, 14, 15, 16, 17, 18 19, 20, 21, 22, or 23, chromosomes; or the detected DMRs are DMRs are from at least about: 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes. In some embodiments, the sperm sample can be obtained from a human male subject at least about: 1 day, 2, days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 51 years, 52 years, 53 years, 54 years, 55 years, 56 years, 57 years, 58 years, 59 years, 60 years, 61 years, 62 years, 63 years, 64 years, 65 years, 66 years, 67 years, 68 years, 69 years, 70 years, 71 years, 72 years, 73 years, 74 years, 75 years, 76 years, 77 years, 78 years, 79 years, 80 years, 81 years, 82 years, 83 years, 84 years, 85 years, 86 years, 87 years, 88 years, 89 years, 90 years, 91 years, 92 years, 93 years, 94 years, 95 years, 96 years, 97 years, 98 years, 99 years, or 100 years of age. In some embodiments, the sperm sample can be obtained from a human male subject of an age ranging from about 15 years to about 80 years of age. In some embodiments, DMRs that are determined and compared, individually, can range from about 100 to about 17000 adjacent nucleotides. In some embodiments, at least a plurality of the DMRs that are determined and compared can comprise a CpG density of less than about 10 CpG per 100 nucleotides. In some embodiments, at least a plurality of the DMRs that are determined and compared can comprise a CpG density of less than about 3 CpG per 100 nucleotides. In some embodiments, at least about: 50, 60, or 70 percent of the DMRs that are determined and compared can be hypermethylated when compared, individually, to individual reference methylation levels of corresponding individual reference DMRs. In some embodiments, at least about: 30, 40, or 50 percent of the DMRs that are determined and compared can be hypomethylated when compared, individually, to individual reference methylation levels of corresponding individual reference DMRs. In some embodiments, a method can further comprise, determining with a computer, a risk of an offspring of the human male subject having a disease or condition. In some embodiments, a method can further comprise, determining with a computer, a severity of autism spectrum disorder of an offspring of the human male subject. In some embodiments, a method can further comprise, determining with a computer, a severity of autism spectrum disorder of the human male subject. In some embodiments, a disease or condition can comprise autism or autism spectrum disorder. In some embodiments, a disease or condition can be selected from the group consisting of a disease related to autism or a neurodegenerative disease, such as Asperger's syndrome. In some embodiments, a method can further comprise performing a further analysis using a computer. In some embodiments, a further analysis can comprise a principle component analysis (PCA), a dendrogram analysis, a machine learning analysis, or any combination thereof. In some embodiments, a further analysis can generate data points, and the data points in the further analysis can be grouped into two spatially distinct categories — a first category which can indicate the subject or an offspring of the subject is at increased risk of having a disease or condition and second category which can indicate the subject or the offspring of the subject is not at increased risk of having the disease or condition.

Also disclosed herein are method, comprising: obtaining a sperm sample from a human male subject; isolating deoxyribonucleic acid (DNA) from the sample; determining a methylation level of a differential DNA methylation region (DMR) comprised in the isolated DNA; and comparing the methylation level of the DMR to a reference level of a corresponding reference DMR. In some embodiments, the comparing can comprise comparing employing a computer comprising a computer processor and computer readable memory comprising computer readable instructions contained thereon. In some embodiments, the determining can comprise a methylated DNA immunoprecipitation (MeDIP), a sequencing, a bisulfite treatment, a bisulfite conversion, a deamination of an unmethylated cytosine base, employing an array, or any combination of these. In some embodiments, a number of determined DMRs can be sufficient to determine, from a process comprising the comparing and employing a computer, whether the human male subject, or an offspring of the human male subject, has or is at increased risk of having autism or autism spectrum disorder, or determine a severity of autism spectrum disorder. In some embodiments, about 90 to about 1000 distinct DMRs can be determined and compared. In some embodiments, about 90 to about 1000 distinct DMRs can be determined and compared, and the about 90 to about 1000 distinct DMRs can be selected from the DMRs in Table 3. In some embodiments, the method can further comprise treating a human male subject or an offspring thereof. In some embodiments, the method can comprise treating the offspring of a human male subject. In some embodiments, treating the offspring can comprise treating at least one cell, treating a human male subject, or treating a sperm cell of the human male subject or a male offspring of the human male subject. In some embodiments, the offspring is less than about 2 years old. In some embodiments, treating can comprise administering an applied behavior analysis, a cognitive behavior therapy, an educational therapy, a joint attention therapy, a nutritional therapy, an occupational therapy, a physical therapy, a social skills training, a speech language therapy, an antipsychotic drug or a salt thereof, risperidone or a salt thereof, aripiprazole or a salt thereof, a selective serotonin re-uptake inhibitor or a salt thereof, citalopram or a salt thereof, escitalopram or a salt thereof, fluoxetine or a salt thereof, fluvoxamine or a salt thereof, paroxetine or a salt thereof, sertraline or a salt thereof, dapoxetine or a salt thereof, indalpine or a salt thereof, zimelidine or a salt thereof, alaproclate or a salt thereof, centpropazine or a salt thereof, femoxetine or a salt thereof, omiloxetine or a salt thereof, panuramine or a salt thereof, seproxetine or a salt thereof, venlafaxine or a salt thereof, clomipramine or a salt thereof, methylphenidate or a salt thereof, mixed amphetamine salts, a psychoactive medication or a salt thereof, a stimulant or a salt thereof, a valproic acid or a salt thereof, phenytoin or a salt thereof, clonazepam or a salt thereof, carbamazepine or a salt thereof, a social skills therapy, speech therapy, supplementing a vitamin or a salt thereof, a mineral or a salt thereof, or both, a restricted diet, a risperidone or a salt thereof, or any combination thereof. In some embodiments, treating can comprise administering a therapeutically effective amount of a pharmaceutical formulation to the subject. In some embodiments, a pharmaceutical formulation can comprise a pharmaceutically acceptable: excipient, diluent, or carrier. In some embodiments, a pharmaceutical formulation can be in unit dose form. In some embodiments, a pharmaceutical formulation can be administered orally, intranasally, by inhalation, sublingually, by injection, by a transdermally, intravenously, subcutaneously, intramuscularly, in an eye, in an ear, in a rectum, intrathecally, or any combination thereof. In some embodiments, a pharmaceutical formulation can be administered in an amount ranging from about 0.0001 to about 100,000 mg of pharmaceutical formulation per kg of subject body weight or offspring of subject body weight. In some embodiments, a method can further comprise transmitting data, a result, or both, via an electronic communication medium.

Also disclosed herein are kits comprising at least about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence present in Table 3; and a container. In some embodiments, the distinct primers or pairs of primers can each further comprise a unique barcode.

Also disclosed herein are kits comprising at least about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct probes, each distinct probe complementary to a distinct DMR sequence present in Table 3; and a container. In some embodiments, distinct probes can further comprise at least one of: a fluorophore, a chromophore, a barcode, or any combination thereof. In some embodiments, each probe can comprise a unique: fluorophore, chromophore, barcode, or any combination thereof. In some embodiments, the probes or the primers may not be bound to an array or a microarray. In some embodiments, the probes or the primers can be bound to an array or a microarray. In some embodiments, wherein the probes, the primers, or both comprise DNA.

Also disclosed herein are methods, comprising: obtaining a sperm sample from a human male subject; isolating deoxyribonucleic acid (DNA) from the sample; fragmenting the DNA; isolating fragmented methylated DNA. In some embodiments, a method can comprise determining a methylation level of a differential DNA methylation region (DMR) comprised in the isolated fragmented methylated DNA; and comparing the methylation level of the DMR to a reference level of a corresponding reference DMR. In some embodiments, the comparing can comprise comparing employing a computer comprising a computer processor and computer readable memory comprising computer readable instructions contained thereon. In some embodiments, the determining can comprise amplification of the isolated fragmented methylated DNA, sequencing the isolated fragmented methylated DNA, an amplicon thereof, or both, employing an array, or any combination of these. In some embodiments, about 100 to about 1000 distinct DMRs can be detected and compared. In some embodiments, the about 100 to about 1000 distinct DMRs can be selected from the DMRs in Table 3. In some embodiments, isolating the fragmented methylated DNA can comprise methylated DNA immunoprecipitation (MeDIP).

Also disclosed herein are methods, comprising: obtaining a sperm sample from a human male subject; isolating deoxyribonucleic acid (DNA) from the sample; fragmenting the DNA; isolating fragmented methylated DNA. In some embodiments, a method can comprise determining a methylation level of a differential DNA methylation region (DMR) comprised in the isolated fragmented methylated DNA; and comparing the methylation level of the DMR to a reference level of a corresponding reference DMR. In some embodiments, the comparing can comprise comparing employing a computer comprising a computer processor and computer readable memory comprising computer readable instructions contained thereon. In some embodiments, the determining can comprise amplification of the isolated fragmented methylated DNA, sequencing the isolated fragmented methylated DNA, an amplicon thereof, or both, employing an array, or any combination of these. In some embodiments, a number of determined DMRs are sufficient to determine, from a process comprising the comparing and employing a computer, whether the human male subject, or an offspring of the human male subject, has or may be at increased risk of having autism or autism spectrum disorder, or determine a severity of autism spectrum disorder. In some embodiments, isolating the fragmented methylated DNA can comprise methylated DNA immunoprecipitation (MeDIP).

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows DMR identifications. FIG. 1A shows autism case versus control sperm DMR analysis. The number of DMRs found using different p-value cutoff thresholds. The all window column shows all DMRs. The multiple window column shows the number of DMRs containing at least two adjacent significant windows and the number of DMRs with each specific number of significant windows at a p-value threshold of 1e-05. FIG. 1B shows autism case versus control patient DMR analysis. The DMR locations on the individual chromosomes. All DMRs at a p-value threshold of p<1e-05 are shown with the arrowhead (triangles) and clusters of DMRs with the black boxes. FIG. 1C shows DMR CpG density in the autism case versus control patient DMRs. The number of DMRs at different CpG densities. All DMRs at a p-value threshold of p<1e-05. FIG. 1D shows autism case versus control patient DMR lengths in kilobases. All DMRs at a p-value threshold of 1e-05 are shown.

FIG. 2 shows DMR associated genes. FIG. 2A shows DMR associated gene categories. DMRs at a p-value threshold p<1e-05 are shown. FIG. 2B shows DMR associated genes and autism. The paternal offspring autism susceptible DMRs previously shown to correlate with autism and associated neurodegenerative disease are presented. DMR associated genes from the current study were compared to genes associated with autism in the published literature using Pathway Studio software (Elsivier, Inc.). Those that were in common are depicted. FIG. 2C shows autism case versus control DMR PCA. PCA analysis for DMRs at p<1e-05. The first three principal components used and samples color code index indicated. The underlying data is the RPKM read depth for all DMRs.

FIG. 3 shows a permutation analysis. The number of DMR for autism case versus control patient comparison for all permutation analyses. The vertical red line shows the number of DMR found in the original analysis. All DMRs are defined using an edgeR p-value threshold of p<1e-05.

### DETAILED DESCRIPTION

### Overview

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Early diagnosis and intervention for Autism Spectrum Disorder (ASD) can be significantly beneficial to the development of the ASD individual and lessens the burden on the families and direct caregivers. The identification of a predictive epigenetic biomarker for ASD from the father's sperm may provide physicians and parents with information that can drive earlier identification of ASD and better care. Presence of an ASD methylation signature in paternal sperm cells may encourage parents and physicians to seek early testing and intervention for children in the early years of life.

ASD has increased over ten-fold over the past several decades, and appears predominantly associated with paternal transmission. Although genetics is anticipated to be a component of ASD etiology, environmental epigenetics is now thought to be an important factor. Epigenetic alterations, such as DNA methylation have been correlated with ASD. The current study was designed to identify a DNA methylation signature in sperm as a potential biomarker to identify paternal offspring autism susceptibility. Sperm samples were obtained from fathers, many undergoing in vitro fertilization (IVF) procedures, that have children with or without autism, and the sperm then assessed for alterations in DNA methylation. Differential DNA methylation regions (DMRs) were identified in the sperm of fathers with autistic children in comparison to those without ASD children. The genomic features and genes associated with the DMRs were identified. The potential sperm DMR biomarker/diagnostic was validated with a blinded test set of individuals. Observations demonstrate a highly significant and reproducible set of 800 DMRs in sperm that can act as a biomarker for paternal offspring autism susceptibility. Ancestral or early life paternal exposures that alter germline epigenetics is anticipated to be a molecular component of ASD etiology.

Although there is both paternal and maternal transmission of ASD, the prevalence of paternal transmission can be higher in most populations. One of the main factors proposed to be involved can be paternal age, with an increased percentage of 28% between 40-49 years and nearly 70% when greater than 50 years of age. Increased paternal age has been associated with epigenetic DNA methylation alterations in sperm, with specific genes associated with autism, and with offspring abnormal behavior. Paternal age associated DNA methylation alterations have been shown to impact offspring health and disease susceptibility. In addition to paternal age effects, ancestral and early life exposures to toxicants, abnormal nutrition and stress can also impact sperm DNA methylation to affect disease susceptibility of offspring. The current disclosure can be directed to examine the father's sperm epigenetics (DNA methylation) in families with or without autistic children.

The prevalence of Autism Spectrum Disorder (ASD) in the United States has doubled since 2000 and currently affects 1 in 59 children, with boys being four-times more likely to be diagnosed (1 in 37 boys are diagnosed with ASD). The increase in ASD prevalence can be due to a combination of factors including increased awareness of the condition in schools and medical environments, and better diagnosis guidelines. Additionally, biological factors such as an increased trend of rising paternal age and improved survival of babies born prematurely have been linked to increased ASD rates. Every individual with ASD can be affected differently however some common challenges associated with ASD include non-verbal communication, difficulty with social engagement, trouble with emotional connection or understanding, and restrictive and repetitive behavior patterns. There is no cure for ASD and, especially for the more severe cases, it is considered a life-long disorder that can place significant emotional and financial burdens on families. A higher incidence of depression and decreased quality of family-life has been associated with familial caregivers of ASD children. Additionally, the burden on caregivers associated with ASD children is persistent from childhood to adolescence and often all the way to adulthood (REF). Medical costs of children with ASD are 4-6 times higher than children without ASD, while behavioral therapies cost families ˜$50,000 per child per year. In 2011 the total cost per year for children with ASD in the US was estimated to cost between $11.5 billion and $60.9 billion. These costs are estimated to grow to $461 billion per year by 2025. Methods and platforms described herein include development of an epigenetic test that may be utilized by a rheumatologist to order prior to prescribing a therapeutic, that can predict which TNF inhibitor a patient is most likely to respond to—and thus may eliminate a trial and error approach for treatment and may ease the debilitating symptoms of RA sufferers. Methods and platforms described herein may use epigenetics as a tool for diagnosis of chronic diseases (such as autoimmune diseases) and prediction of therapeutic response.

Recent research has shown dramatic benefits for early diagnosis and treatment of ASD. Early behavior, communication, and social therapies can greatly improve the associated skills leading to reduced care needs and financial burden through adolescence and adulthood. The skills taught from Early and Intensive Behavioral Intervention (EIBI) have been shown to last for more than a decade and lead to significantly decreased symptoms of ASD. To maximize these benefits intervention needs to start as early as possible, before a child's brain has fully developed. ASD can be diagnosed as early as 12-18 months, and EIBI at these ages has been shown to have the most dramatic benefits. Unfortunately, the average age of diagnosis in the US can be 4-5 years old, after the child's brain has already significantly developed and created permanent connections. Better awareness of ASD risk factors and symptoms can be important for early intervention and improving long-term outcomes for language, daily living skills, social behavior, and cognition.

The cause of ASD is unknown, however, research has identified both genetic and environmental factors that are associated with increased occurrences of ASD. Increased risk has been linked to families with a history of ASD, increased paternal age, prenatal chemical exposures, and preterm birth. Developing a reliable test for ASD prediction in offspring can both help uncover the causes and empower parents to seek earlier diagnosis and treatment.

With advancing molecular diagnostic tools, the identification of novel genetic and epigenetic markers for ASD is becoming a realistic option. Significant funding has driven research on the genetic basis of, and diagnostics for, ASD. Through large-scale genome-wide-analyses several genetic variants have been shown to substantially contribute to the susceptibility of ASD, however these fall short of being predictive for most of the population. Disclosed herein are methods of detecting and treating autism, ASD and similar disorders.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present disclosure.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values can be described in the application and claims, unless otherwise stated the term “about” meaning within an acceptable error range for the particular value should be assumed. Also, where ranges, subranges, or both, of values can be provided, the ranges or subranges can include the endpoints of the ranges or subranges. The terms “substantially”, “substantially no”, “substantially not”, “substantially free”, and “approximately” can be used when describing a magnitude, a position or both to indicate that the value described can be within a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

As used herein, the terms “treating, ” “ treatment” and the like are used herein to mean obtaining a desired pharmacologic and/or physiologic effect. The effect may be prophylactic in terms of completely or partially preventing a disease, disorder, or condition or sign or symptom thereof, and/or may be therapeutic in terms of a partial or complete cure for a disease or condition. In some instances, a disease or condition can comprise Autism Spectrum Disorder, Autism, or any combination thereof. In some instances, an individual can be treated therapeutically, such therapeutic treatment can cause a partial or a complete cure for the disease or disorder. In some cases, therapeutic treatment can comprise a pharmaceutical composition disclosed herein, a behavioral therapy (e.g. psychological therapy), or a combination of both. In some instances, a treatment can reverse an adverse effect attributable to the disease or disorder. In some cases, treating can comprise treating the offspring of a male subject. In some instances, treating can comprise treating at least one cell, treating a human male subject, or treating a sperm cell of the human male subject or a male offspring of the human male subject. In some cases, treating can comprise treating an offspring that is less than about: 1, 2, 3, 4, 5, 6, 7, 8, 9, 10, 11, 12, 13, 14, 15, 16, 17, 18, 19 or 20 years of age. In some cases, treating can comprise treating an offspring that is more than about: 1, 2, 3, 4, 5, 6, 7, 8, 9, 10, 11, 12, 13, 14, 15, 16, 17, 18, 19 or 20 years of age. In some cases, the treatment can stabilize the disease or disorder. In some cases, the treatment can delay progression of the disease or disorder. In some instances, the treatment can cause regression of the disease or disorder. In some cases, a treatment's effect can be measured. In some cases, measurements can be compared before and after administration of the composition. For example, a subject can have Autism Diagnostic Observation Schedule (ADOS) and its Severity Score recorded before therapy and compared to the ADOS after treatment to show improvement in ASD. In some instances, measurements can be compared to a standard.

An “effective amount” can be an amount of a therapeutic agent sufficient to achieve an intended purpose. An effective amount of a composition to treat or ameliorate a disease (e.g. ASD) can be an amount of the composition sufficient to reduce or remove the symptoms of the disorder.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

Epigenetics, as used herein, generally refers to “molecular factors and processes around DNA that regulate genome activity independent of DNA sequence and are mitotically stable.” The molecular factors and processes currently known are DNA methylation, histone modifications, chromatin structural changes, non-coding RNA, and RNA methylation. When the epigenetic alterations become programmed in the germ cells (sperm or egg), they have the ability to promote the epigenetic transgenerational inheritance of disease and phenotypic alterations. Environmental factors that promote these early life epigenetic alterations have the ability to promote epigenetic inheritance to subsequent generations, and dramatically increase disease susceptibility and prevalence. The current study was designed to use a genome-wide approach and develop a potential paternal sperm biomarker for offspring with autism susceptibility.

The term “subject,” as used herein, generally refers to any individual that has, may have, or may be suspected of having a disease condition (e.g., Autism Spectrum Disorder (ASD)). The subject may be an animal. The animal can be a mammal, such as a human, non-human primate, a rodent such as a mouse or rat, a dog, a cat, pig, sheep, or rabbit. Animals can be fish, reptiles, or others Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as ASD. The subject may be a patient being treated for a disease, such as ASD. The subject may be predisposed to a risk of developing a disease such as ASD. The subject may be in remission from a disease, such as ASD. The subject may not have ASD. The subject may be healthy.

The term “sample,” as used herein, generally refers to any sample of a subject (such as a blood sample, a plasma sample, a urine sample, a sperm sample, a vaginal swab, a sweat sample, a saliva sample, a biological fluid sample, a cell-free sample, a tissue sample, a buccal swab, or a nasal swab). Genomic data may be obtained from the sample. A sample may be a sample suspected or confirmed of having a disease or condition such as ASD. A sample may be a sample removed from a subject, such as a tissue brushing, a swabbing, a tissue biopsy, an excised tissue, a fine needle aspirate, a tissue washing, a cytology specimen, a bronchoscopy, or any combination thereof.

The term “increased risk” in the context of developing or having ASD, as used herein, generally refers to an increased risk or probability associated with the occurrence of ASD in a subject. An increased risk of developing ASD can include a first occurrence of the condition in a subject or can include subsequent occurrences, such as a second, third, fourth, or subsequent occurrence. An increased risk of developing ASD can include a) a risk of developing the condition for a first time, b) a risk of developing the condition in the future, c) a risk of being predisposed to developing the condition in the subject's lifetime, or d) a risk of being predisposed to developing the condition as an infant, adolescent, or adult.

As used herein, a “biosimilar” or a “biosimilar product” may refer to a biological product that is licensed based on a showing that it is substantially similar to an FDA-approved biological product, known as a reference product, and has no clinically meaningful differences in terms of safety and effectiveness from the reference product. Only minor differences in clinically inactive components may be allowable in biosimilar products. A “biosimilar” of an approved reference product/biological drug refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In some embodiments, the biosimilar biological product and reference product utilize the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held may meet standards designed to assure that the biological product continues to be safe, pure, and potent. The reference product may be approved in at least one of the U.S., Europe, or Japan. In some embodiments, a response rate of human subjects administered the biosimilar product can be 50%-150% of the response rate of human subjects administered the reference product. For example, the response rate of human subjects administered the biosimilar product can be 50%-100%, 50%-110%, 50%-120%, 50%-130%, 50%-140%, 50%-150%, 60%-100%, 60%-110%, 60%-120%, 60%-130%, 60%-140%, 60%-150%, 70%-100%, 70%-110%, 70%-120%, 70%-130%, 70%-140%, 70%-150%, 80%-100%, 80%-110%, 80%-120%, 80%-130%, 80%-140%, 80%-150%, 90%-100%, 90%-110%, 90%-120%, 90%-130%, 90%-140%, 90%-150%, 100%-110%, 100%-120%, 100%-130%, 100%-140%, 100%-150%, 110%-120%, 110%-130%, 110%-140%, 110%-150%, 120%-130%, 120%-140%, 120%-150%, 130%-140%, 130%-150%, or 140%-150% of the response rate of human subjects administered the reference product. In some embodiments, a biosimilar product and a reference product can utilize the same mechanism or mechanisms of action for the condition or conditions of use prescribed, recommended, or suggested in the proposed labeling, but only to extent the mechanism or mechanisms are known for the reference product. To obtain approval for biosimilar drugs, studies and data of structure, function, animal toxicity, pharmacokinetics, pharmacodynamics, immunogenicity, and clinical safety and efficacy may be needed. A biosimilar may also be known as a follow-on biologic or a subsequent entry biologic. In some embodiments, a biosimilar product may be substantially similar to the reference product notwithstanding minor different in clinically inactive components.

As used herein, a “interchangeable biological product” may refer to a biosimilar of an FDA-approved reference product and may meet additional standards for interchangeability. In some embodiments, an interchangeable biological product can, for example, produce the same clinical result as the reference product in any given subject. In some embodiments, an interchangeable product may contain the same amount of the same active ingredients, may possess comparable pharmacokinetic properties, may have the same clinically significant characteristics, and may be administered in the same way as the reference compound. In some embodiments, an interchangeable product can be a biosimilar product that meets additional standards for interchangeability. In some embodiments, an interchangeable product can produce the same clinical result as a reference product in all the reference product's licensed conditions of use. In some embodiments, an interchangeable product can be substituted for the reference product by a pharmacist without the intervention of the health care provider who prescribed the reference product. In some embodiments, when administered more than once to an individual, the risk in terms of safety or diminished efficacy of alternating or switching between use of the biological product and the reference product is not greater than the risk of using the reference product without such alternation or switch. In some embodiments, an interchangeable product can be a regulatory agency approved product. In some embodiments, a response rate of human subjects administered the interchangeable product can be 80%-120% of the response rate of human subjects administered the reference product. For example, the response rate of human subjects administered the interchangeable product can be 80%-100%, 80%-110%, 80%-120%, 90%-100%, 90%-110%, 90%-120%, 100%-110%, 100%-120%, or 110%-120 of the response rate of human subjects administered the reference product.

The term “sequencing” as used herein, may comprise high-throughput sequencing, next-gen sequencing, Maxam-Gilbert sequencing, massively parallel signature sequencing, Polony sequencing, 454 pyrosequencing, pH sequencing, Sanger sequencing (chain termination), Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, sequencing-by-hybridization, sequencing by synthesis, sequencing-by-ligation, or any combination thereof. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system. In some cases, a nucleic acid of a sample may be sequenced without an associated label or tag. In some cases, a nucleic acid of a sample may be sequenced, the nucleic acid of which may have a label or tag associated with it.

### Methods of Detection and Treatment

Methods described herein can be used to detect a neurodegenerative disease, autism or autism spectrum disorder. In some embodiments, a method can comprise obtaining a sperm sample from a human male subject; isolating deoxyribonucleic acid (DNA) from the sample; determining a methylation level of a differential DNA methylation region (DMR) comprised in the isolated DNA; and comparing the methylation level of the DMR to a reference level of a corresponding reference DMR. In some instances, DNA can be fragmented. In some instances, a methylation level can comprise hypomethylation, hypermethylation, or no change in methylation. In some cases, comparing can comprise comparing employing a computer comprising a computer processor and computer readable memory comprising computer readable instructions contained thereon. In some cases, the determining can comprise a methylated DNA immunoprecipitation (MeDIP), a sequencing, a bisulfite treatment, a bisulfite conversion, a deamination of an unmethylated cytosine base, employing an array, or any combination of these. In some instances, MeDIP can be used to isolate methylated DNA from a sample. In some cases, determining can comprise amplification of an isolated fragmented methylated DNA, sequencing the isolated fragmented methylated DNA, an amplicon thereof, or both, employing an array (e.g. a microarray), or any combination of these. In some cases, a number of determined DMRs can be sufficient to determine, from a process comprising comparing and employing a computer, whether the human male subject, or an offspring of the human male subject, has or is at increased risk of having autism or autism spectrum disorder, or determine a severity of autism spectrum disorder. In some cases, about 90 to about 1000 distinct DMRs are detected and compared. In the distinct DMRs can be selected from the DMRs in Table 3. In some cases, about 200 to about 1000 distinct DMRs, about 300 to about 1000 distinct DMRs, about 400 to about 1000 distinct DMRs, about 500 to about 1000 distinct DMRs, about 600 to about 1000 distinct DMRs, about 700 to about 1000 distinct DMRs, about 800 to about 1000, or about 900 to about 1000 distinct DMRs can be detected. In some cases, more than 1000 distinct DMRs can be detected, for example about: 1500, 2000, 2500, 3000, 3500, 4500, 5000, 5500, 6000, 6500, 7000, 7500, 8000, 8500, 9000, 9500 or more distinct DMRs can be detected. In some cases, about: 1000 to about 2000, 2000 to about 3000, 3000 to about 5000, 4000 to about 7000, 5000 to about 7500, 6000 to about 8500, or 8500 to about 10000 distinct DMRs can be detected. In some cases, less than about 200 distinct DMRs can be detected for example about: 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 distinct DMRs can be detected. In some cases, about: 1 to about 10, 10 to about 50, 25 to about 75, 40 to about 100, 80 to about 140, 120 to about 180, or 140 to about 200 distinct DMRs can be detected.

In some embodiments, the detected DMRs can comprise DMRs from at least about: 1, 2, 3, 4, 5, 6, 7, 8, 9, 10, 11, 12, 13, 14, 15, 16, 17, 18 19, 20, 21, 22, or 23, chromosomes. In some cases, the detected DMRs can be DMRs are from at least about: 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes. In some cases, the detected DMRs can be detected from any part of a genome. In some cases, the detected DMRs can be from a specific part of the genome, for example a specific chromosome. In some cases, the DMRs that are determined and compared, individually, range from about: 10 to about 1000, 25 to about 1500, 50 to about 500, 1000 to about 2500, 100 to about 17000, 2500 to about 7500, 5000 to about 20000, 7500 to about 15000 or 10000 to about 25000 adjacent nucleotides. In some embodiments, at least a plurality of the DMRs that can be determined and compared comprise a CpG density of less than about: 1, 2, 3, 4, 5, 6, 7, 8, 9, 10, 11, 12, 13, 14, 15, 16, 17, 18, 19, 20, 21, 22, 23, 24, or 25 CpG per 100 nucleotides. In some embodiments, at least a plurality of the DMRs that can be determined and compared comprise a CpG density of more than about: 1, 2, 3, 4, 5, 6, 7, 8, 9, 10, 11, 12, 13, 14, 15, 16, 17, 18, 19, 20, 21, 22, 23, 24, or 25 CpG per 100 nucleotides.

In some embodiments about: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 percent of the DMRs that are determined and compared can be hypermethylated when compared, individually, to individual reference methylation levels of corresponding individual reference DMRs. In some embodiments about: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 percent of the DMRs that are determined and compared can be hypomethylated when compared, individually, to individual reference methylation levels of corresponding individual reference DMRs.

In some embodiments, a method can comprise, determining with a computer, a risk of an offspring of a human male subject having a disease or condition. In some cases, a disease or condition can comprise autism or autism spectrum disorder. In some cases, a disease or condition can be a neurodegenerative disease such as Asperger's syndrome or any disease or condition related to autism or autism spectrum disorder. In some cases, a method can comprise determining with a computer, a severity of autism spectrum disorder of an offspring of a human male subject. In some cases, a method can comprise using a computer for further analysis. In some cases, further analysis can comprise a principle component analysis (PCA), a dendrogram analysis, a machine learning analysis, or any combination thereof. In some cases, further analysis can generate data points, and the data points can be grouped into two spatially distinct categories—a first category which indicates the subject or an offspring of the subject is at increased risk of having a disease or condition and second category which indicates the subject or the offspring of the subject is not at increased risk of having a disease or condition. In some cases, a method can comprise transmitting data, a result or both via an electronic communication medium.

In some embodiments, a cell (e.g. a sperm sample) can be obtained from a subject. In some cases, a subject can be a human male or a human female subject. In some cases, a cell can be a stem cell, a cartilage cell, a bone cell, a blood cell, a muscle cell, a fat cell, a skin cell, a nerve cell, an endothelial cell, an epithelial cell, a sex cell, a pancreatic cell, a cancer cell, or any combination thereof. In some cases, a cell can be a sperm cell. In some cases, a cell sample can be obtained from a subject at least about: 1 day, 2, days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 51 years, 52 years, 53 years, 54 years, 55 years, 56 years, 57 years, 58 years, 59 years, 60 years, 61 years, 62 years, 63 years, 64 years, 65 years, 66 years, 67 years, 68 years, 69 years, 70 years, 71 years, 72 years, 73 years, 74 years, 75 years, 76 years, 77 years, 78 years, 79 years, 80 years, 81 years, 82 years, 83 years, 84 years, 85 years, 86 years, 87 years, 88 years, 89 years, 90 years, 91 years, 92 years, 93 years, 94 years, 95 years, 96 years, 97 years, 98 years, 99 years, or 100 years of age. In some cases, a sample can be obtained from a subject who can be about: 1 day to about 1 week old, 1 week to about 5 weeks old, 5 weeks to about 12 months old, 1 year to about 6 years old, 6 years to about 100 years old, 6 years to about 12 years old, 12 years to about 60 years old, 15 years to about 80 years old, 20 years to about 70 years old, or 30 years to about 120 years old.

An epigenetic modification may comprise a 5-methylated base, such as a 5-methylated cytosine (5-mC). An epigenetic modification may comprise a 5-hydroxymethylated base, such as a 5-hydroxymethylated cytosine (5-hmC). An epigenetic modification may comprise a 5-formylated base, such as a 5-formylated cytosine (5-fC). An epigenetic modification may comprise a 5-carboxylated base or a salt thereof, such as a 5-carboxylated cytosine (5-caC). A nucleic acid sequence may comprise an epigenetic modification. A nucleic acid sequence may comprise a plurality of epigenetic modifications. A nucleic acid sequence may comprise an epigenetic modification positioned within a CG site, a CpG island, a CpG desert (i.e., a nucleotide sequence region with lower CpG density) or a combination thereof. A nucleic acid sequence may comprise different epigenetic modifications, such as a methylated base, a hydroxymethylated base, a formylated base, a carboxylic acid containing base or a salt thereof, a plurality of any of these, or any combination thereof.

The use of a sperm epigenetic biomarkers for paternal offspring autism susceptibility could be used in an assisted reproduction setting. Although genetic tests are common in assisted reproduction and preimplantation diagnostics, epigenetic analysis may be less common. Sperm DNA methylation diagnostics have been proposed for use in assisted reproduction. The availability of a sperm DNA methylation biomarker for offspring autism susceptibility would allow improved clinical management and early treatment options to be considered. A genome-wide analysis of DNA methylation alterations in sperm from fathers with or without autistic children was used to identify a potential sperm epigenetic biomarker for paternal offspring autism susceptibility.

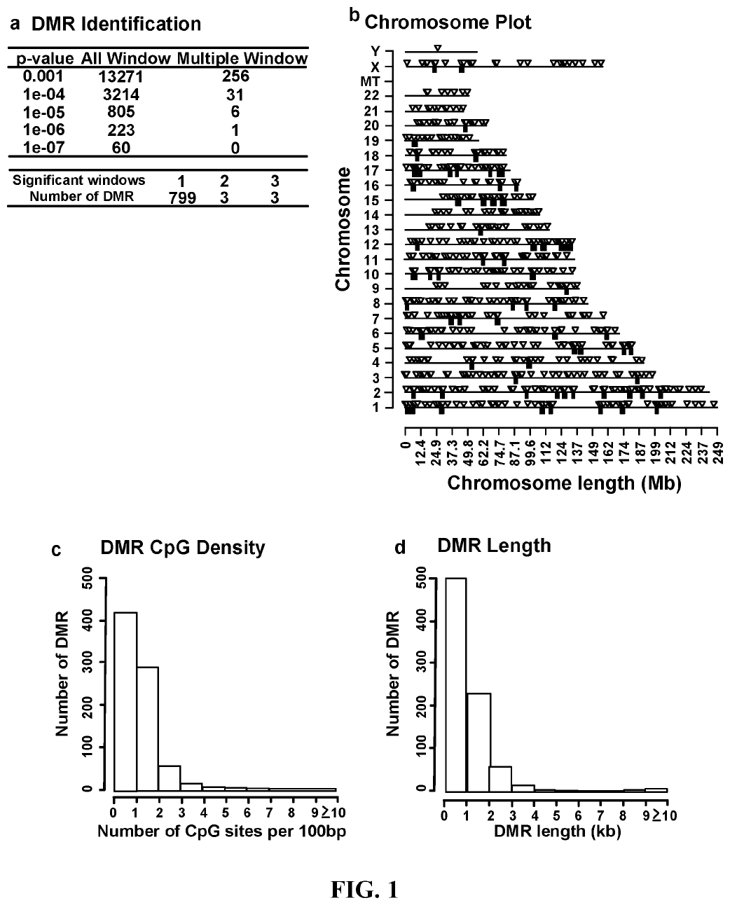

FIG. 1B shows data linking paternal-sperm DNA methylation to ASD risk in offspring. In a cohort of fathers with offspring diagnosed with ASD and a matching control group of fathers with healthy offspring, a consistent and unique DNA methylation pattern was found at 223 locations (p=1e-06) throughout the genome.

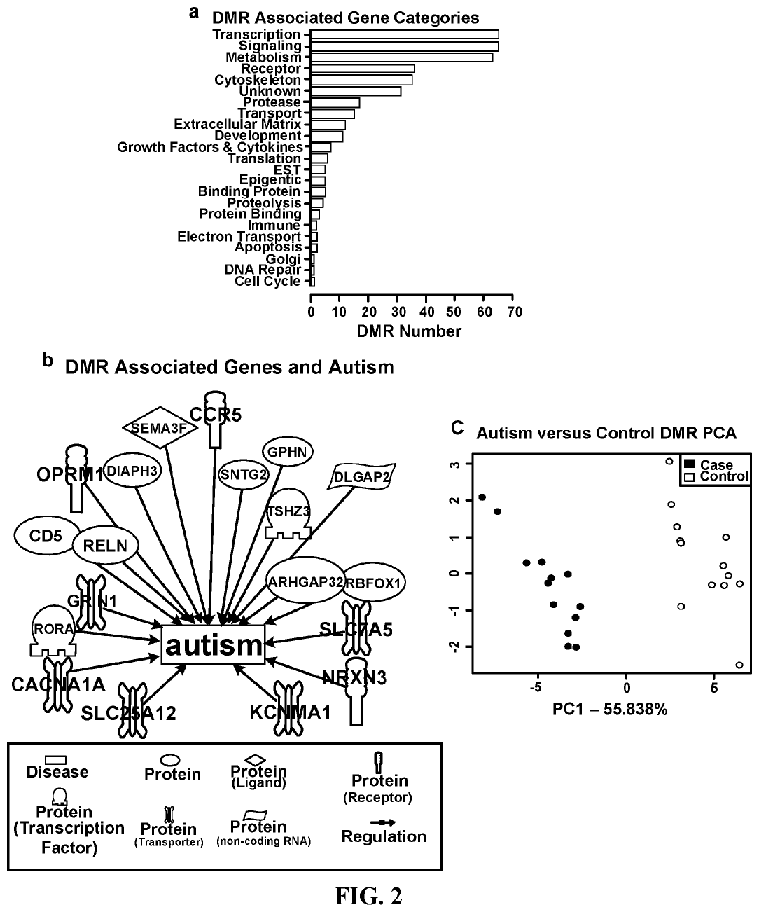

Referring to FIG. 2C, it shows control and study participant/subjects methylation data separated and clustered into their own cohorts in an unsupervised statistical analysis, PCA plot, showing evidence of a unique methylation pattern between groups. Further, FIG. 2A outlines the gene categories that had differential methylation patterns in the study samples versus the control samples where changes in DNA methylation significantly change in genes associated with transcription, signaling and metabolism.

In some embodiments, a method can comprise treating a disease or condition. In some cases, a method can comprise treating a male subject or the offspring of the male subject thereof. In some cases, the method can comprise treating at least one cell such as a sperm cell. In some cases, the method can comprise treating a human male subject or the offspring of the human male subject. In some cases, treating can comprise administering a therapy. In some cases, a therapy can comprise a applied behavior analysis, a cognitive behavior therapy, an educational therapy, a joint attention therapy, a nutritional therapy, an occupational therapy, a physical therapy, a social skills training, a social skills therapy, speech therapy, a speech language therapy, or any combination thereof. In some cases, treating can comprise administering an antipsychotic drug or a salt thereof, risperidone or a salt thereof, aripiprazole or a salt thereof, a selective serotonin re-uptake inhibitor or a salt thereof, citalopram or a salt thereof, escitalopram or a salt thereof, fluoxetine or a salt thereof, fluvoxamine or a salt thereof, paroxetine or a salt thereof, sertraline or a salt thereof, dapoxetine or a salt thereof, indalpine or a salt thereof, zimelidine or a salt thereof, alaproclate or a salt thereof, centpropazine or a salt thereof, femoxetine or a salt thereof, omiloxetine or a salt thereof, panuramine or a salt thereof, seproxetine or a salt thereof, venlafaxine or a salt thereof, clomipramine or a salt thereof, methylphenidate or a salt thereof, mixed amphetamine salts, a psychoactive medication or a salt thereof, a stimulant or a salt thereof, a valproic acid or a salt thereof, phenytoin or a salt thereof, clonazepam or a salt thereof, carbamazepine or a salt thereof, risperidone or a salt thereof, an attention-deficit/hyperactivity disorder medication, an amphetamine mixed salts or any combination thereof. In some cases, treating can comprise administering clozapine or a salt thereof, haloperidol or a salt thereof, oxytocin or a salt thereof, secretin or a salt thereof, bumetanide or a salt thereof, memantine or a salt thereof, rivastigmine or a salt thereof, mirtazapine or a salt thereof, melatonin or a salt thereof. In some cases, treatment can comprise supplementing a vitamin or a salt thereof, a mineral or a salt thereof, or both, a restricted diet, or any combination thereof. In some cases, treating can comprise administering a therapeutically effective amount of a pharmaceutical formulation (e.g. pharmaceutical composition) to a subject. In some cases, a pharmaceutical formulation can be administered in unit dose form. In some case a pharmaceutical formulation can be administered orally, intranasally, by inhalation, sublingually, by injection, by a transdermally, intravenously, subcutaneously, intramuscularly, in an eye, in an ear, in a rectum, intrathecally, or any combination thereof.

In some embodiments, a pharmaceutical formulation can be administered in an amount ranging from about: 0.0001 mg to about 100,000 mg, 0.001 mg to about 10,000 mg, 0.01 mg to about 1,000 mg, 0.1 mg to about 100 mg, or about 1 mg to about 10 mg of pharmaceutical formulation per kg of subject body weight or offspring of subject body weight.

In some embodiments, compositions disclosed herein can be in unit dose forms or multiple dose forms. For example, a pharmaceutical composition described herein can be in unit dose form. Unit dose forms, as used herein, refer to physically discrete units suitable for administration to human or non-human subjects (e.g. pets) and packaged individually. Each unit dose can contain a predetermined quantity of an active ingredient(s) that may be sufficient to produce the desired therapeutic effect in association with pharmaceutical carriers, diluents, excipients or any combination thereof. Examples of unit dose forms can include ampules, syringes, and individually packaged tablets and capsules.

In some embodiments, a composition disclosed herein can be formulated as a pharmaceutical composition. In some cases, a composition can comprise an excipient, a diluent, a carrier or any combination thereof. In some cases, the compositions can be made by mixing a composition described herein, and a pharmaceutically acceptable excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent or any combination thereof. In some instances, the excipient comprising one or more of cellulose, disodium hydrogen phosphate, hydroxypropyl cellulose, hypromellose, lactose, mannitol, or sodium lauryl sulfate. In some instances, the compositions further comprise glyceryl monostearate 40-50, hydroxypropyl cellulose, hypromellose, magnesium stearate, methacrylic acid copolymer type C, polysorbate 80, sugar spheres, talc, or triethyl citrate. In some instances, a composition can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, or yellow ferric oxide. In some instances, a composition can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate. Examples of carriers for the composition include any degradable, partially degradable or non-degradable and generally biocompatible polymer, e.g., polystirex, polypropylene, polyethylene, polacrilex, poly-lactic acid (PLA), polyglycolic acid (PGA) and/or poly-lactic polyglycolic acid (PGLA), e.g., in the form or a liquid, matrix, or bead. In some instances, a binder can comprise starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides or any combination thereof.

In some embodiments, a pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some embodiments, a salt can include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N, N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. In some cases, a salt can comprise a pharmaceutically acceptable salt. In some instances, a salt of a polypeptide or derivative thereof or a compound can be a Zwitterionic salt

Administration disclosed herein to a subject in need of treatment can be achieved by, for example and not by way of limitation, oral administration, topical administration, intravenous administration, inhalation administration, or any combination thereof. In some cases, delivery can include injection, catheterization, gastrostomy tube administration, intraosseous administration, ocular administration, otic administration, transdermal administration, oral administration, rectal administration, nasal administration, intravaginal administration, intracavernous administration, transurethral administration, sublingual administration, or a combination thereof. Delivery can include direct application to the affect tissue or region of the body. Delivery can include a parenchymal injection, an intra-thecal injection, an intra-ventricular injection, or an intra-cisternal injection. A composition provided herein can be administered by any method. A method of administration can be by intraarterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump or a combination thereof. In some cases, delivery can be by an enema, an eye drop, a nasal spray, an ear drop, or any combination thereof.

In some embodiments, a healthcare provider can administer a composition herein to a subject in need thereof. In some cases, a healthcare provider or the subject can administer the method of detecting a DMR.

Administration of a composition or therapy disclosed herein can be performed for a duration of at least about at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000 days consecutive or nonconsecutive days. In some cases, the composition or therapy can be administered for life. In some cases, administration of the composition or therapy described herein can be from about 1 to about 30 days, from about 1 to about 60 days, from about 1 to about 90 days, from about 1 to about 300 days, from about 1 to about 3000 days, from about 30 day to about 90 days, from about 60 days to about 900 days, from about 30 days to about 900 days, or from about 90 days to about 1500 days. In some cases, administration of the composition described herein can be from about: 1 week to about 5 weeks, 1 month to about 12 months, 1 year to about 3 years, 2 years to about 8 years, 3 years to about 10 years, 10 years to about 50 years, 15 years to about 40 years, 25 years to about 100 years, 30 years to about 75 years, 60 years to about 110 years, or about 50 years to about 130 years.

Administration of a composition or therapy disclosed herein can be performed for a duration of at least about: 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a day, once a week, or once a month for the lifetime of a subject Administration can be performed repeatedly over a substantial portion of a subject's life, such as once a day, once a week, or once a month for at least about: 1 year, 5 years, 10 years, 15 years, 20 years, 25 years, 30 years, or more.

Administration of composition or therapy disclosed herein can be performed at least about: 1, 2, 3, 4, 5, 6, 7, 8, 9, 10, 11, 12, 13, 14, 15, 16, 17, 18, 19, 20, 21, 22, 23, or 24 times a in a 24-hour period. In some instances, administration can comprise administration of a pharmaceutical formulation, a supplement, a therapy or any combination thereof. In some cases, administration of a composition can be performed continuously throughout a 24-hour period. In some cases, administration of composition or therapy disclosed herein can be performed at least about: 1, 2, 3, 4, 5, 6, 7, 8, 9, 10, 11, 12, 13, 14, 15, 16, 17, 18, 19, 20, or 21 times a week. In some cases, administration of a composition or therapy disclosed herein can be performed at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, or more times a month. In some cases, a composition can be administered as a single dose or as divided doses. In some cases, the compositions described herein can be administered at a first time point and a second time point. In some cases, a composition can be administered such that a first administration can be administered before the other with a difference in administration time of about: 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some cases, a subject may have diagnosed prior to treatment. In some cases, a method described herein can further comprise diagnosing a subject.

### Kits

Also described herein are kits comprising distinct primers or pairs of primers and a container. In some cases, a kit can comprise about more than about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, 1000, 1100, 1200, 1300, 1400, 1500, 1600, 1700, 1800, 1900 or 2000 distinct primers or pairs of primers. In some cases, a kit can comprise about less than about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, 1000, 1100, 1200, 1300, 1400, 1500, 1600, 1700, 1800, 1900 or 2000 distinct primers or pairs of primers. In some cases, each distinct primer or pairs of primers can comprise a distinct sequence complementary to a distinct DMR sequence or a region comprising a distinct DMR sequence present in Table 3. In some cases, a kit can comprise about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, 1100, 1200, 1300, 1400, 1500, 1600, 1700, 1800, 1900 or 2000 distinct probes. In some cases, a distinct probe can be complementary to a distinct DMR sequence or region comprising a DMR sequence in Table 3. In some cases, a probe can comprise at least one of a fluorophore, a chromophore, a barcode or a combination thereof. In some cases, a primer or a pair of primers can comprise a unique barcode. In some cases, a probe, a primer, or a pair of primers may not be bound do an array or a microarray. In some cases, a probe, a primer, or a pair of primers can be bound do an array or a microarray. In some cases, a probe and/or primer can comprise a nucleic acid. In some cases, a nucleic acid can comprise DNA.

EXAMPLES

### Example 1

### Results

Paternal males with children affected by autism (case) or without (control) were recruited and paternal sperm samples were collected at the Andrology Laboratory of IVIRMA Clinic in Valencia, Spain. The sperm sample was collected upon enrollment. Thirty-two patients were enrolled, which included thirteen in the control group, thirteen in the autism case group, and six for the blinded test group. The differences (mean±SD) between the semen analysis for both control and case group are shown in Table 1. Observations from the groups showed no significant difference in sperm volume, concentration, or sperm concentration between the groups. Progressive sperm motility was greater in the autism case group, with no difference in non-progressive sperm motility, Table 1. The motile percentage was higher in the control group, and no difference was observed in the total motile sperm count. One of the control samples IVI 14 had a very high sperm count of 396.62 million that was outside two standard deviations of the mean (2±SD), so the analysis was redone without this sample. When the IVI 14 sample was not used in the analysis, the total sperm number was increased in the autism case group (p<0.02), and the total motile sperm count (Time) was increased in the autism case group (p<0.017), as well as the progressive spermatozoa (%) (p<0.019) and immotile % (p<0.019) parameters. The ethnicity of all the fathers was Caucasian. The date of the patient sperm collection, age of the father, and age of the case father at pregnancy are all provided in Table 1. All the autistic children were males. The human subjects' approval was obtained prior to the initiation of the study and approved by the Ethics Committee of IVIRMA Valencia, with code, #1311-VLC-136-FC.

TABLE 1
Sperm Samples and Clinical Analysis

| | | | | | | Paternal Sperm Analysis | | | | | | |
|---|---|---|---|---|---|---|---|---|---|---|---|---|
| Study Sample | | | | | | | | | | | | TOTAL |
| | | FATHER | FATHER | | OFFSPRING | | | | | | | MOTILE |
| | | AGE | AGE | COLLECTION | AUTISM | | | TOTAL OF | PROGRESSIVE | NON-PROGRESSIVE | | SPERM |
| | AGE | (yrs) UPON | (yrs) AT | DATE | CASE/ | VOLUME | CONCENTRATION | SPERMATOZOA | SPERMATOZOA | SPERMATOZOA | INMOTILE | COUNT |
| SAMPLE | (yrs) | COLLECTION | PREGNANCY | OF SAMPLE | CONTROL | (mL) | (mill/mL) | (mill) | (%) | (%) | (%) | (Time) |
| IVI 1 | 42 | 42 | 31 | Jul. 27, 2015 | CASE | 2.2 | 83 | 182.6 | 58 | 11 | 31 | 105.91 |
| IVI 2 | 44 | 45 | 43 | Jul. 28, 2015 | CASE | 1.5 | 38 | 57 | 42 | 11 | 47 | 23.94 |
| IVI 3 | 41 | 41 | 38 | Jul. 29, 2015 | CASE | 3 | 68 | 204 | 44 | 11 | 45 | 89.76 |
| IVI 4 | 38 | 38 | 34 | Jul. 29, 2015 | CASE | 3.4 | 66 | 224.4 | 35 | 11 | 54 | 78.54 |
| IVI 5 | 37 | 37 | 33 | Jul. 30, 2015 | CASE | 1.4 | 10 | 14 | 50 | 14 | 36 | 7 |
| IVI 6 | 39 | 49 | 40 | Aug. 4, 2015 | CASE | 2.2 | 23 | 50.6 | 31 | 26 | 43 | 15.69 |
| IVI 7 | 41 | 41 | 31 | Aug. 12, 2015 | CASE | 6 | 47 | 282 | 69 | 14 | 17 | 194.58 |
| IVI 8 | 42 | 42 | 32 | Aug. 14, 2015 | CASE | 2.5 | 9 | 227.5 | 60 | 15 | 25 | 136.5 |
| IVI 9 | 45 | 45 | 35 | Sep. 9, 2015 | CASE | 3.5 | 87 | 304.5 | 50 | 13 | 37 | 152.25 |
| IVI 10 | — | 39 | 31 | Sep. 28, 2015 | CASE | 3 | 33.3 | 99.9 | 44 | 9 | 47 | 43.96 |
| IVI 11 | — | 45 | 39 | Dec. 21, 2015 | CASE | 4 | 120 | 480 | 50 | 12 | 38 | 240 |
| IVI 12 | 35 | 37 | 24 | Sep. 5, 2017 | CASE | 5.4 | 36.3 | 196.02 | 43 | 3 | 54 | 84.29 |
| IVI 13 | 46 | 46 | 35 | Mar. 7, 2016 | CASE | 4.2 | 17.6 | 73.92 | 60 | 17 | 23 | 44.35 |
| IVI 14 | 40 | 40 | | Oct. 11, 16 | CONTROL | 2.8 | 141.65 | 396.62 | 55 | 10 | 35 | 218.14 |
| IVI 15 | 41 | 41 | | Oct. 11, 2016 | CONTROL | 1 | 34.3 | 34.3 | 31 | 18 | 51 | 10.63 |
| IVI 16 | 46 | 46 | | Oct. 17, 2016 | CONTROL | 2.2 | 5.5 | 12.1 | 29 | 3 | 68 | 3.51 |
| IVI 17 | 44 | 44 | | Oct. 20, 2016 | CONTROL | 4.8 | 10.5 | 50.4 | 42 | 12 | 46 | 21.17 |
| IVI 18 | 38 | 38 | | Oct. 21, 2016 | CONTROL | 3.2 | 1.6 | 5.12 | 28 | 13 | 59 | 1.43 |
| IVI 19 | 36 | 36 | | Nov. 3, 2016 | CONTROL | 6.8 | 4 | 95.2 | 32 | 3 | 65 | 30.46 |
| IVI 20 | 41 | 41 | | Mar. 22, 2017 | CONTROL | 2.2 | 91.8 | 201.96 | 49 | 14 | 37 | 98.96 |
| IVI 21 | 42 | 42 | | May 23, 2017 | CONTROL | 1.4 | 42.1 | 58.94 | 37 | 23 | 40 | 21.81 |
| IVI 22 | 37 | 37 | | Sep. 6, 2017 | CONTROL | 4.2 | 5 | 226.8 | 52 | 9 | 39 | 117.94 |
| IVI 23 | 43 | 43 | | Sep. 6, 2017 | CONTROL | 1.8 | 13 | 23.4 | 23 | 20 | 57 | 5.38 |
| IVI 24 | 54 | 54 | | Sep. 15, 2017 | CONTROL | 2.5 | 52 | 130 | 48 | 7 | 45 | 62.4 |
| IVI 25 | 38 | 38 | | Sep. 15, 2017 | CONTROL | 5.5 | 1.7 | 9.35 | 11 | 8 | 81 | 1.03 |
| IVI 26 | 43 | 43 | | Sep. 22, 2017 | CONTROL | 1.5 | 96 | 144 | 35 | 17 | 48 | 50.4 |

| | | | | | | | |
|---|---|---|---|---|---|---|---|
| Mean ± SD Offspring Autism Case | 3 ± 1 | 55 ± 33 | 184 ± 128 | 49 ± 11 | 13 ± 5 | 38 ± 12 | 94 ± 71 |
| Mean ± SD Offspring Control | 3 ± 2 | 43 ± 44 | 107 ± 114 | 36 ± 13 | 12 ± 6 | 52 ± 14 | 49 ± 63 |
| Statistical Comparison (Case vs. Control), Not Significant | NS | NS | NS | P < 0.01 | NS | P < 0.01 | NS |
| (NS) p > 0.05 | | | | | | | |

Individual patient sperm samples from the collection upon enrollment were prepared for sperm analysis, and an aliquot taken, and flash frozen with liquid nitrogen and stored at −20 C. until shipment on dry ice for the epigenetic analysis. The DNA was extracted from the sperm then fragmented for a methylated DNA immunoprecipitation (MeDIP) analysis in order to identify differential DNA methylated regions (DMRs). The MeDIP is a genome-wide analysis examining 95% of the genome comprising low density CpG regions in comparison to the less than 5% of the genome of high density regions and CpG islands. The MeDIP DNA was then prepared for next generation DNA sequencing by creating individual patient sequencing libraries. Samples were then sequenced for bioinformatic analysis, as described in the Supplemental Methods section. A comparison of the sequences between the control (non-autism children) and case (autism children) patient sperm samples identified DMRs, FIG. 1A. At a p-value of p<1e-05 there were 805 DMRs identified with the majority being a single 1 kb window with fewer (i.e. six) having multiple adjacent 1 kb windows involved. The DMRs at a number of p-values are presented for p<001 to p<1e-07, FIG. 1A. The DMRs at p<1e-05 were used for subsequent data analysis, and a list of these DMRs with various genomic features are presented in Table 3. Observations demonstrate that males with autistic children have a sperm DMR signature that is distinct from males without autistic children (control).

TABLE 3
Exemplary list of DMRs
DMR List Case vs. Control p < 1e−05

| | | | | | # Sig | | | | CpG | | |
|---|---|---|---|---|---|---|---|---|---|---|---|
| DMR Name | Chr | Start | Stop | Length | Win | minP | maxLFC | CpG # | Density | Gene Annotation | Gene Category |
| DMR1: 386001 | 1 | 386001 | 389000 | 3000 | 1 | 6.10E−06 | 0.8139908 | 31 | 1.033 | AL732372.2 | |
| DMR1: 517001 | 1 | 517001 | 518000 | 1000 | 1 | 3.58E−06 | 0.9372704 | 10 | 1 | AL732372.2; RF00026 | |
| DMR1: 824001 | 1 | 824001 | 825000 | 1000 | 1 | 2.83E−07 | −1.2835435 | 6 | 0.6 | AL669831.3; AL669831.4; | |
| | | | | | | | | | | FAM87B; LINC01128; LINC00115 | |
| DMR1: 2656001 | 1 | 2656001 | 2667000 | 11000 | 3 | 1.10E−08 | 1.0405831 | 267 | 2.427 | TTC34 | |
| DMR1: 2668001 | 1 | 2668001 | 2683000 | 15000 | 3 | 4.42E−08 | 0.8755558 | 363 | 2.42 | TTC34 | |
| DMR1: 3571001 | 1 | 3571001 | 3572000 | 1000 | 1 | 1.60E−06 | 0.9307299 | 10 | 1 | MEGF6 | Growth Factors & Cytokines |
| DMR1: 3920001 | 1 | 3920001 | 3921000 | 1000 | 1 | 2.77E−06 | 0.6803401 | 8 | 0.8 | LINC01134 | |
| DMR1: 6258001 | 1 | 6258001 | 6259000 | 1000 | 1 | 5.33E−09 | 0.6549455 | 24 | 2.4 | GPR153; ACOT7 | Metabolism |
| DMR1: 6522001 | 1 | 6522001 | 6523000 | 1000 | 1 | 9.22E−06 | 0.3394789 | 35 | 3.5 | PLEKHG5; NOL9 | Signaling; Transcription |
| DMR1: 7222001 | 1 | 7222001 | 7224000 | 2000 | 1 | 7.86E−07 | −0.4398214 | 34 | 1.7 | CAMTA1; RNU1-8P | Transcription |
| DMR1: 7966001 | 1 | 7966001 | 7967000 | 1000 | 1 | 4.45E−07 | −0.571204 | 18 | 1.8 | PARK7 | Development |
| DMR1: 9642001 | 1 | 9642001 | 9644000 | 2000 | 1 | 1.02E−06 | 0.705812 | 33 | 1.65 | PIK3CD; PIK3CD-AS1 | Signaling |
| DMR1: 15866001 | 1 | 15866001 | 15867000 | 1000 | 1 | 4.64E−07 | 0.6211895 | 14 | 1.4 | SPEN | Transcription |
| DMR1: 19808001 | 1 | 19808001 | 19811000 | 3000 | 1 | 8.97E−06 | −0.4319503 | 33 | 1.1 | TMCO4; RNF186; AL391883.1 | |
| DMR1: 24159001 | 1 | 24159001 | 24162000 | 3000 | 1 | 3.90E−06 | 0.7355099 | 43 | 1.433 | IFNLR1 | Receptor |
| DMR1: 24226001 | 1 | 24226001 | 24228000 | 2000 | 1 | 1.52E−06 | 0.6173313 | 26 | 1.3 | | |
| DMR1: 26621001 | 1 | 26621001 | 26622000 | 1000 | 1 | 5.27E−06 | 0.7579737 | 69 | 6.9 | | |
| DMR1: 28784001 | 1 | 28784001 | 28785000 | 1000 | 1 | 8.21E−06 | 0.5491186 | 25 | 2.5 | | |
| DMR1: 28907001 | 1 | 28907001 | 28909000 | 2000 | 1 | 9.03E−07 | 0.5713446 | 44 | 2.2 | EPB41 | |
| DMR1: 29480001 | 1 | 29480001 | 29481000 | 1000 | 1 | 6.96E−07 | 0.670635 | 10 | 1 | AL671862.1 | |
| DMR1: 30010001 | 1 | 30010001 | 30011000 | 1000 | 1 | 7.09E−07 | −0.5386825 | 20 | 2 | LINC01648 | |
| DMR1: 33804001 | 1 | 33804001 | 33806000 | 2000 | 1 | 3.15E−07 | −0.5887044 | 12 | 0.6 | CSMD2 | Unknown |
| DMR1: 41502001 | 1 | 41502001 | 41503000 | 1000 | 1 | 9.96E−06 | −0.5315307 | 9 | 0.9 | HIVEP3 | Transcription |
| DMR1: 46799001 | 1 | 46799001 | 46800000 | 1000 | 1 | 8.31E−06 | −0.5854623 | 13 | 1.3 | CYP4B1 | Metabolism |
| DMR1: 54305001 | 1 | 54305001 | 54307000 | 2000 | 1 | 6.42E−06 | −0.4431958 | 30 | 1.5 | SSBP3 | Translation |
| DMR1: 59530001 | 1 | 59530001 | 59532000 | 2000 | 1 | 2.16E−06 | −0.7317057 | 9 | 0.45 | FGGY | Signaling |
| DMR1: 61708001 | 1 | 61708001 | 61709000 | 1000 | 1 | 2.21E−06 | 0.8628242 | 6 | 0.6 | TM2D1 | Unknown |
| DMR1: 69382001 | 1 | 69382001 | 69383000 | 1000 | 1 | 7.36E−06 | −0.5941512 | 10 | 1 | | |
| DMR1: 70268001 | 1 | 70268001 | 70269000 | 1000 | 1 | 5.34E−06 | 0.5314653 | 12 | 1.2 | ANKRD13C | |
| DMR1: 72494001 | 1 | 72494001 | 72495000 | 1000 | 1 | 8.03E−06 | −0.625482 | 1 | 0.1 | | |
| DMR1: 74449001 | 1 | 74449001 | 74450000 | 1000 | 1 | 1.94E−06 | −0.4828113 | 8 | 0.8 | FPGT-TNNI3K; TNNI3K | Transcription |
| DMR1: 79458001 | 1 | 79458001 | 79459000 | 1000 | 1 | 2.48E−06 | −0.5341593 | 9 | 0.9 | | |
| DMR1: 79677001 | 1 | 79677001 | 79678000 | 1000 | 1 | 4.17E−06 | −0.6074266 | 4 | 0.4 | | |
| DMR1: 89368001 | 1 | 89368001 | 89369000 | 1000 | 1 | 6.02E−06 | −0.6720726 | 6 | 0.6 | GBP6 | Signaling |
| DMR1: 97504001 | 1 | 97504001 | 97506000 | 2000 | 1 | 4.69E−06 | −0.8910787 | 12 | 0.6 | DPYD | Metabolism |
| DMR1: 103356001 | 1 | 103356001 | 103358000 | 2000 | 1 | 7.28E−08 | −0.6163035 | 13 | 0.65 | | |
| DMR1: 109039001 | 1 | 109039001 | 109040000 | 1000 | 1 | 5.02E−08 | 0.6790758 | 18 | 1.8 | WDR47; BX679664.1; RANP5 | |
| DMR1: 109276001 | 1 | 109276001 | 109277000 | 1000 | 1 | 7.42E−07 | 0.8406167 | 15 | 1.5 | CELSR2; PSRC1 | Cytoskeleton |
| DMR1: 109537001 | 1 | 109537001 | 109538000 | 1000 | 1 | 2.85E−06 | 0.6361615 | 13 | 1.3 | GPR61; AL355310.3 | Signaling |
| DMR1: 112075001 | 1 | 112075001 | 112077000 | 2000 | 1 | 8.92E−07 | 0.7714121 | 5 | 0.25 | | |
| DMR1: 115225001 | 1 | 115225001 | 115227000 | 2000 | 1 | 4.15E−06 | −0.5591425 | 21 | 1.05 | | |
| DMR1: 115384001 | 1 | 115384001 | 115385000 | 1000 | 1 | 2.98E−06 | −0.9033439 | 1 | 0.1 | | |
| DMR1: 116921001 | 1 | 116921001 | 116923000 | 2000 | 1 | 6.04E−06 | −0.4536816 | 20 | 1 | PTGFRN | |
| DMR1: 117487001 | 1 | 117487001 | 117488000 | 1000 | 1 | 7.49E−08 | −0.670835 | 6 | 0.6 | MAN1A2; AL157902.2 | Metabolism |
| DMR1: 154605001 | 1 | 154605001 | 154607000 | 2000 | 1 | 9.58E−06 | 1.088414 | 23 | 1.15 | ADAR | Transcription |
| DMR1: 156166001 | 1 | 156166001 | 156167000 | 1000 | 1 | 3.15E−06 | 0.8921821 | 8 | 0.8 | SEMA4A | Development |
| DMR1: 156171001 | 1 | 156171001 | 156174000 | 3000 | 1 | 5.84E−06 | 0.5794197 | 48 | 1.6 | SEMA4A | Development |
| DMR1: 158269001 | 1 | 158269001 | 158270000 | 1000 | 1 | 2.47E−06 | −0.5083161 | 10 | 1 | HMGN1P5 | |
| DMR1: 161306001 | 1 | 161306001 | 161307000 | 1000 | 1 | 6.21E−07 | 1.1274576 | 35 | 3.5 | MPZ; SDHC | Extracellular Matrix |
| DMR1: 164199001 | 1 | 164199001 | 164200000 | 1000 | 1 | 4.12E−06 | −0.4587256 | 7 | 0.7 | | |
| DMR1: 166045001 | 1 | 166045001 | 166047000 | 2000 | 1 | 5.38E−06 | −0.586517 | 11 | 0.55 | RNA5SP64 | |
| DMR1: 170280001 | 1 | 170280001 | 170281000 | 1000 | 1 | 2.39E−06 | −0.4550262 | 6 | 0.6 | LINC01142 | |
| DMR1: 173100001 | 1 | 173100001 | 173101000 | 1000 | 1 | 1.46E−06 | −0.5881991 | 5 | 0.5 | | |
| DMR1: 173156001 | 1 | 173156001 | 173157000 | 1000 | 1 | 1.79E−07 | −0.4918186 | 6 | 0.6 | | |
| DMR1: 174089001 | 1 | 174089001 | 174090000 | 1000 | 1 | 3.08E−07 | −0.5918141 | 5 | 0.5 | RPL30P1 | |
| DMR1: 176750001 | 1 | 176750001 | 176751000 | 1000 | 1 | 7.81E−07 | 0.8580497 | 10 | 1 | PAPPA2 | |
| DMR1: 178399001 | 1 | 178399001 | 178400000 | 1000 | 1 | 6.92E−06 | −0.4565437 | 8 | 0.8 | RASAL2 | Signaling |
| DMR1: 193063001 | 1 | 193063001 | 193064000 | 1000 | 1 | 9.19E−06 | −0.6469959 | 6 | 0.6 | UCHL5; SCARNA18B; RO60 | Protease |
| DMR1: 199919001 | 1 | 199919001 | 199920000 | 1000 | 1 | 7.47E−06 | −0.4324768 | 10 | 1 | AL445687.2 | |
| DMR1: 201006001 | 1 | 201006001 | 201008000 | 2000 | 1 | 1.27E−06 | −0.3748639 | 22 | 1.1 | KIF21B | Cytoskeleton |
| DMR1: 201696001 | 1 | 201696001 | 201697000 | 1000 | 1 | 5.80E−06 | −0.5161451 | 23 | 2.3 | NAV1; IPO9-AS1 | |
| DMR1: 204002001 | 1 | 204002001 | 204003000 | 1000 | 1 | 4.95E−06 | 0.9292689 | 10 | 1 | | |
| DMR1: 205229001 | 1 | 205229001 | 205231000 | 2000 | 1 | 2.97E−07 | −0.7229921 | 46 | 2.3 | TMCC2; AC093422.2 | Unknown |
| DMR1: 206732001 | 1 | 206732001 | 206734000 | 2000 | 1 | 7.16E−06 | −0.3181144 | 39 | 1.95 | MAPKAPK2 | Signaling |
| DMR1: 207843001 | 1 | 207843001 | 207844000 | 1000 | 1 | 6.98E−06 | −0.4149983 | 12 | 1.2 | MIR29B2CHG | |
| DMR2: 204061001 | 2 | 204061001 | 204062000 | 1000 | 1 | 1.33E−09 | −0.7914435 | 9 | 0.9 | AC009965.2 | |
| DMR2: 206667001 | 2 | 206667001 | 206669000 | 2000 | 1 | 3.27E−08 | −0.7418124 | 15 | 0.75 | DYTN | |
| DMR2: 208215001 | 2 | 208215001 | 208216000 | 1000 | 1 | 8.26E−06 | 0.6347569 | 15 | 1.5 | RPSAP27; TPT1P2 | |
| DMR2: 211716001 | 2 | 211716001 | 211717000 | 1000 | 1 | 2.11E−06 | 0.4703591 | 30 | 3 | ERBB4 | Signaling |
| DMR2: 216710001 | 2 | 216710001 | 216713000 | 3000 | 1 | 1.59E−07 | −0.5847288 | 39 | 1.3 | AC007563.2 | |
| DMR2: 218561001 | 2 | 218561001 | 218562000 | 1000 | 1 | 4.87E−07 | −0.4202055 | 12 | 1.2 | USP37; CNOT9 | Protease |
| DMR2: 226806001 | 2 | 226806001 | 226808000 | 2000 | 1 | 7.02E−07 | −0.6563554 | 15 | 0.75 | IRS1; AC010735.2; AC010735.1 | Unknown |
| DMR2: 231272001 | 2 | 231272001 | 231273000 | 1000 | 1 | 2.75E−06 | 0.5428854 | 20 | 2 | ARMC9 | |
| DMR2: 235963001 | 2 | 235963001 | 235965000 | 2000 | 1 | 3.57E−07 | −0.5756016 | 36 | 1.8 | AGAP1 | Signaling |
| DMR3: 304001 | 3 | 304001 | 305000 | 1000 | 1 | 2.59E−06 | −0.662561 | 7 | 0.7 | CHL1; RPS8P6 | Extracellular Matrix |
| DMR3: 1034001 | 3 | 1034001 | 1035000 | 1000 | 1 | 1.10E−07 | −0.9882 | 5 | 0.5 | | |
| DMR3: 9344001 | 3 | 9344001 | 9345000 | 1000 | 1 | 7.73E−07 | 0.5947837 | 25 | 2.5 | SRGAP3; AC026191.1; | Signaling |
| | | | | | | | | | | PGAM1P4; THUMPD3-AS1 | |
| DMR3: 12368001 | 3 | 12368001 | 12370000 | 2000 | 1 | 3.30E−06 | 0.7976002 | 17 | 0.85 | PPARG | Receptor |
| DMR3: 14334001 | 3 | 14334001 | 14335000 | 1000 | 1 | 2.11E−07 | −0.5358308 | 12 | 1.2 | | |
| DMR3: 15593001 | 3 | 15593001 | 15596000 | 3000 | 1 | 8.85E−06 | 0.6773648 | 45 | 1.5 | HACL1; BTD | Metabolism |
| DMR3: 19593001 | 3 | 19593001 | 19594000 | 1000 | 1 | 9.42E−06 | −0.379851 | 9 | 0.9 | | |
| DMR3: 20600001 | 3 | 20600001 | 20601000 | 1000 | 1 | 6.74E−07 | −0.5380999 | 10 | 1 | SGO1-AS1 | |
| DMR3: 32765001 | 3 | 32765001 | 32767000 | 2000 | 1 | 5.67E−06 | 1.1020184 | 36 | 1.8 | CNOT10 | |
| DMR3: 34173001 | 3 | 34173001 | 34174000 | 1000 | 1 | 8.35E−06 | −0.733452 | 4 | 0.4 | LINC01811 | |
| DMR3: 46373001 | 3 | 46373001 | 46376000 | 3000 | 1 | 3.79E−07 | 0.7776259 | 30 | 1 | AC098613.1; CCR5 | Growth Factors & Cytokines |
| DMR3: 48201001 | 3 | 48201001 | 48202000 | 1000 | 1 | 9.15E−06 | 0.5055743 | 29 | 2.9 | MIR4443 | |
| DMR3: 50192001 | 3 | 50192001 | 50193000 | 1000 | 1 | 6.23E−06 | 1.3249619 | 25 | 2.5 | SEMA3F; GNAT1 | Growth Factors & Cytokines; |
| | | | | | | | | | | | Signaling |
| DMR3: 51110001 | 3 | 51110001 | 51111000 | 1000 | 1 | 6.32E−06 | −0.5088342 | 4 | 0.4 | DOCK3 | Signaling |
| DMR3: 54078001 | 3 | 54078001 | 54079000 | 1000 | 1 | 9.12E−06 | −0.9024898 | 10 | 1 | | |
| DMR3: 59570001 | 3 | 59570001 | 59572000 | 2000 | 1 | 1.87E−06 | 0.5355753 | 16 | 0.8 | | |
| DMR3: 64181001 | 3 | 64181001 | 64183000 | 2000 | 1 | 2.93E−06 | −0.5960076 | 13 | 0.65 | PRICKLE2; PRDX3P4; PRICKLE2- | Cytoskeleton |
| | | | | | | | | | | AS3 | |
| DMR3: 68392001 | 3 | 68392001 | 68394000 | 2000 | 1 | 4.11E−06 | −0.5435105 | 15 | 0.75 | FAM19A1 | |
| DMR3: 73021001 | 3 | 73021001 | 73022000 | 1000 | 1 | 5.31E−06 | 0.9559614 | 6 | 0.6 | PPP4R2; RNU7-19P | |
| DMR3: 74120001 | 3 | 74120001 | 74121000 | 1000 | 1 | 2.34E−08 | −1.4543578 | 5 | 0.5 | | |
| DMR3: 78490001 | 3 | 78490001 | 78491000 | 1000 | 1 | 7.61E−09 | −0.6011024 | 11 | 1.1 | | |
| DMR3: 87649001 | 3 | 87649001 | 87651000 | 2000 | 1 | 2.10E−06 | −0.8331352 | 8 | 0.4 | AC108749.1 | |
| DMR3: 88836001 | 3 | 88836001 | 88837000 | 1000 | 1 | 9.61E−07 | −0.9823663 | 6 | 0.6 | | |
| DMR3: 89157001 | 3 | 89157001 | 89158000 | 1000 | 1 | 2.80E−07 | −1.0778311 | 8 | 0.8 | EPHA3 | Receptor |
| DMR3: 91375001 | 3 | 91375001 | 91376000 | 1000 | 1 | 3.91E−07 | 1.1716095 | 3 | 0.3 | ABBA01000935.2 | |
| DMR3: 91549001 | 3 | 91549001 | 91554000 | 5000 | 2 | 7.47E−07 | 1.0363492 | 79 | 1.58 | | |
| DMR3: 93705001 | 3 | 93705001 | 93714000 | 9000 | 3 | 3.57E−08 | 0.8443159 | 153 | 1.7 | | |
| DMR3: 100255001 | 3 | 100255001 | 100256000 | 1000 | 1 | 1.04E−07 | 1.0378051 | 5 | 0.5 | TBC1D23 | |
| DMR3: 100289001 | 3 | 100289001 | 100290000 | 1000 | 1 | 7.50E−06 | 0.8356997 | 9 | 0.9 | TBC1D23 | |
| DMR3: 110714001 | 3 | 110714001 | 110715000 | 1000 | 1 | 5.50E−06 | −0.9557768 | 5 | 0.5 | | |
| DMR3: 117669001 | 3 | 117669001 | 117670000 | 1000 | 1 | 1.41E−06 | −0.7118383 | 10 | 1 | AC092691.1; AC092691.2; | |
| | | | | | | | | | | LINC02024 | |
| DMR3: 122419001 | 3 | 122419001 | 122420000 | 1000 | 1 | 2.99E−07 | 0.6840997 | 40 | 4 | FAM162A; WDR5B; AC083798.2; | Unknown; Metabolism |
| | | | | | | | | | | AC083798.1; KPNA1 | |
| DMR3: 129294001 | 3 | 129294001 | 129295000 | 1000 | 1 | 3.67E−07 | 0.6314323 | 16 | 1.6 | HMCES | |
| DMR3: 130403001 | 3 | 130403001 | 130405000 | 2000 | 1 | 3.20E−06 | −0.8355836 | 9 | 0.45 | COL6A5 | |
| DMR3: 134740001 | 3 | 134740001 | 134741000 | 1000 | 1 | 1.67E−06 | 0.5250261 | 21 | 2.1 | EPHB1 | Receptor |
| DMR3: 139029001 | 3 | 139029001 | 139030000 | 1000 | 1 | 8.83E−07 | −0.4752568 | 9 | 0.9 | MRPS22; PRR23B | Transcription |
| DMR3: 143232001 | 3 | 143232001 | 143233000 | 1000 | 1 | 9.77E−06 | −0.7744006 | 3 | 0.3 | | |
| DMR3: 151846001 | 3 | 151846001 | 151847000 | 1000 | 1 | 7.08E−06 | −0.8926749 | 13 | 1.3 | AADACL2-AS1 | |
| DMR3: 155545001 | 3 | 155545001 | 155546000 | 1000 | 1 | 4.90E−06 | −0.4916974 | 7 | 0.7 | PLCH1 | Metabolism |
| DMR3: 159021001 | 3 | 159021001 | 159022000 | 1000 | 1 | 3.82E−07 | −1.0881578 | 2 | 0.2 | IQCJ-SCHIP1; IQCJ | |
| DMR3: 168445001 | 3 | 168445001 | 168449000 | 4000 | 1 | 1.36E−06 | −0.8900465 | 22 | 0.55 | EGFEM1P | |
| DMR3: 170743001 | 3 | 170743001 | 170744000 | 1000 | 1 | 4.53E−06 | −0.6188246 | 7 | 0.7 | AC026316.4; SLC7A14- | |
| | | | | | | | | | | AS1; AC026316.2 | |
| DMR3: 181107001 | 3 | 181107001 | 181108000 | 1000 | 1 | 9.79E−06 | −0.5737711 | 13 | 1.3 | SOX2-OT | |
| DMR3: 184810001 | 3 | 184810001 | 184811000 | 1000 | 1 | 5.88E−06 | 0.774526 | 8 | 0.8 | VPS8 | |
| DMR3: 185345001 | 3 | 185345001 | 185346000 | 1000 | 1 | 5.97E−06 | −0.3851038 | 11 | 1.1 | MAP3K13 | Signaling |
| DMR3: 185844001 | 3 | 185844001 | 185845000 | 1000 | 1 | 3.88E−06 | −0.6385158 | 11 | 1.1 | | |
| DMR3: 188581001 | 3 | 188581001 | 188582000 | 1000 | 1 | 6.75E−07 | −0.4045768 | 8 | 0.8 | LPP | Cytoskeleton |
| DMR3: 192432001 | 3 | 192432001 | 192434000 | 2000 | 1 | 2.43E−06 | −0.5818597 | 20 | 1 | FGF12 | Growth Factors & Cytokines |
| DMR3: 196945001 | 3 | 196945001 | 196946000 | 1000 | 1 | 9.74E−06 | 0.5237731 | 20 | 2 | NCBP2; NCBP2-AS1; NCBP2- | Transcription; Metabolism |
| | | | | | | | | | | AS2; PIGZ | |
| DMR4: 3759001 | 4 | 3759001 | 3760000 | 1000 | 1 | 6.31E−06 | −0.5600381 | 29 | 2.9 | LINC02600; ADRA2C | Receptor |
| DMR4: 6951001 | 4 | 6951001 | 6953000 | 2000 | 1 | 1.99E−06 | −0.4179546 | 26 | 1.3 | TBC1D14 | Signaling |
| DMR4: 7448001 | 4 | 7448001 | 7451000 | 3000 | 1 | 9.05E−06 | 0.8706796 | 45 | 1.5 | SORCS2; MIR4274 | Receptor |
| DMR4: 9862001 | 4 | 9862001 | 9863000 | 1000 | 1 | 2.44E−06 | −0.708581 | 8 | 0.8 | SLC2A9 | Metabolism |
| DMR4: 10066001 | 4 | 10066001 | 10067000 | 1000 | 1 | 5.57E−07 | −0.5455646 | 11 | 1.1 | AC005674.2; WDR1 | Unknown |
| DMR4: 12097001 | 4 | 12097001 | 12098000 | 1000 | 1 | 7.77E−06 | −0.8038681 | 4 | 0.4 | | |
| DMR4: 13176001 | 4 | 13176001 | 13177000 | 1000 | 1 | 1.61E−06 | −0.5710104 | 8 | 0.8 | | |
| DMR4: 16890001 | 4 | 16890001 | 16891000 | 1000 | 1 | 5.75E−07 | −0.7572532 | 10 | 1 | LDB2 | Transcription |
| DMR4: 18209001 | 4 | 18209001 | 18212000 | 3000 | 1 | 2.17E−07 | −0.6757687 | 11 | 0.367 | | |
| DMR4: 49222001 | 4 | 49222001 | 49223000 | 1000 | 1 | 1.02E−06 | 0.5127587 | 6 | 0.6 | AC118282.2; AC118282.4 | |
| DMR4: 49224001 | 4 | 49224001 | 49226000 | 2000 | 1 | 4.26E−07 | 0.611876 | 15 | 0.75 | AC118282.4; SNX18P23 | |
| DMR4: 52666001 | 4 | 52666001 | 52667000 | 1000 | 1 | 3.84E−06 | 0.5704389 | 2 | 0.2 | USP46; USP46-AS1 | Proteolysis |
| DMR4: 52973001 | 4 | 52973001 | 52974000 | 1000 | 1 | 5.63E−06 | −0.6395201 | 5 | 0.5 | SCFD2 | Unknown |
| DMR4: 53467001 | 4 | 53467001 | 53469000 | 2000 | 1 | 5.10E−06 | −0.7313163 | 22 | 1.1 | FIP1L1; AC058822.1; LNX1 | Cytoskeleton |
| DMR4: 63099001 | 4 | 63099001 | 63100000 | 1000 | 1 | 1.24E−06 | −0.6469376 | 4 | 0.4 | | |
| DMR4: 72705001 | 4 | 72705001 | 72706000 | 1000 | 1 | 1.95E−06 | 0.7554899 | 12 | 1.2 | | |
| DMR4: 75551001 | 4 | 75551001 | 75552000 | 1000 | 1 | 6.14E−07 | 0.5856654 | 18 | 1.8 | THAP6; ODAPH | Transcription |
| DMR4: 76025001 | 4 | 76025001 | 76026000 | 1000 | 1 | 3.02E−06 | −0.5129691 | 7 | 0.7 | ART3; CXCL10; CXCL11 | Metabolism; Growth Factors& |
| | | | | | | | | | | | Cytokines |
| DMR4: 87346001 | 4 | 87346001 | 87347000 | 1000 | 1 | 6.15E−07 | 0.7495587 | 15 | 1.5 | AC108516.2; HSD17B11 | Metabolism |
| DMR4: 88979001 | 4 | 88979001 | 88980000 | 1000 | 1 | 5.00E−06 | 0.7908192 | 8 | 0.8 | FAM13A | |
| DMR4: 94591001 | 4 | 94591001 | 94593000 | 2000 | 1 | 1.71E−06 | −0.7403106 | 15 | 0.75 | PDLIM5 | Cytoskeleton |
| DMR4: 98055001 | 4 | 98055001 | 98057000 | 2000 | 1 | 2.10E−06 | −0.8233235 | 13 | 0.65 | STPG2; DUTP8 | Development |
| DMR4: 98282001 | 4 | 98282001 | 98284000 | 2000 | 1 | 2.73E−07 | −0.5139398 | 14 | 0.7 | RAP1GDS1 | Signaling |
| DMR4: 99064001 | 4 | 99064001 | 99066000 | 2000 | 1 | 1.24E−06 | 0.8542533 | 22 | 1.1 | METAP1; AC019131.2; ADH5 | Protease; Metabolism |
| DMR4: 100090001 | 4 | 100090001 | 100091000 | 1000 | 1 | 1.58E−06 | −0.6226641 | 4 | 0.4 | AC097460.1 | |
| DMR4: 100204001 | 4 | 100204001 | 100205000 | 1000 | 1 | 1.90E−06 | 0.9039055 | 21 | 2.1 | AC097460.1; AP001961.1 | |
| DMR4: 104066001 | 4 | 104066001 | 104067000 | 1000 | 1 | 2.28E−07 | −0.8211243 | 9 | 0.9 | | |
| DMR4: 107484001 | 4 | 107484001 | 107485000 | 1000 | 1 | 3.40E−07 | −0.6626713 | 5 | 0.5 | | |
| DMR4: 122751001 | 4 | 122751001 | 122752000 | 1000 | 1 | 1.91E−06 | −0.5261898 | 6 | 0.6 | BBS12 | |
| DMR4: 128916001 | 4 | 128916001 | 128917000 | 1000 | 1 | 7.29E−06 | −0.905045 | 6 | 0.6 | SCLT1 | Metabolism |
| DMR4: 140013001 | 4 | 140013001 | 140015000 | 2000 | 1 | 6.49E−06 | −0.8846538 | 14 | 0.7 | MAML3 | EST |
| DMR4: 143686001 | 4 | 143686001 | 143687000 | 1000 | 1 | 2.54E−06 | −0.5027995 | 10 | 1 | AC107223.1; FREM3 | Transport |
| DMR4: 147213001 | 4 | 147213001 | 147215000 | 2000 | 1 | 2.20E−06 | −0.8128533 | 8 | 0.4 | | |
| DMR4: 153111001 | 4 | 153111001 | 153112000 | 1000 | 1 | 2.55E−06 | −0.6121456 | 14 | 1.4 | | |
| DMR4: 164321001 | 4 | 164321001 | 164322000 | 1000 | 1 | 2.74E−06 | −0.7249916 | 5 | 0.5 | 1-Mar | Metabolism |
| DMR4: 165109001 | 4 | 165109001 | 165111000 | 2000 | 1 | 9.92E−06 | 0.7641435 | 19 | 0.95 | TMEM192 | Unknown |
| DMR4: 184480001 | 4 | 184480001 | 184481000 | 1000 | 1 | 1.01E−06 | −0.4423459 | 6 | 0.6 | IRF2; AC099343.2; AC099343.3 | Transcription |
| DMR4: 188636001 | 4 | 188636001 | 188637000 | 1000 | 1 | 4.63E−06 | 0.681319 | 15 | 1.5 | LINC01060; AC093909.3 | |
| DMR5: 696001 | 5 | 696001 | 699000 | 3000 | 1 | 2.05E−06 | −0.6667814 | 48 | 1.6 | TPPP | Cytoskeleton |
| DMR5: 1461001 | 5 | 1461001 | 1463000 | 2000 | 1 | 6.49E−06 | −1.7427536 | 53 | 2.65 | LPCAT1 | Metabolism |
| DMR5: 3128001 | 5 | 3128001 | 3130000 | 2000 | 1 | 3.94E−06 | −0.6158025 | 51 | 2.55 | | |
| DMR5: 4492001 | 5 | 4492001 | 4493000 | 1000 | 1 | 7.33E−08 | −0.7392452 | 15 | 1.5 | AC106799.2 | |
| DMR5: 20445001 | 5 | 20445001 | 20447000 | 2000 | 1 | 4.42E−06 | −0.5958123 | 13 | 0.65 | CDH18 | Cytoskeleton |
| DMR5: 27224001 | 5 | 27224001 | 27225000 | 1000 | 1 | 1.94E−08 | −0.8761978 | 5 | 0.5 | PURPL | |
| DMR5: 32070001 | 5 | 32070001 | 32071000 | 1000 | 1 | 1.02E−06 | 0.6963497 | 17 | 1.7 | PDZD2 | |
| DMR5: 37976001 | 5 | 37976001 | 37978000 | 2000 | 1 | 5.29E−06 | −0.7481109 | 16 | 0.8 | AC034226.1 | |
| DMR5: 39122001 | 5 | 39122001 | 39123000 | 1000 | 1 | 8.38E−06 | −0.4437573 | 7 | 0.7 | FYB1 | |
| DMR5: 44758001 | 5 | 44758001 | 44759000 | 1000 | 1 | 9.05E−06 | −0.4511642 | 9 | 0.9 | MRPS30-DT; AC093297.1 | |
| DMR5: 46433001 | 5 | 46433001 | 46436000 | 3000 | 1 | 4.69E−06 | −0.5484316 | 37 | 1.233 | | |
| DMR5: 54849001 | 5 | 54849001 | 54850000 | 1000 | 1 | 9.09E−08 | −0.7133944 | 8 | 0.8 | AC112198.2; AC112198.1 | |
| DMR5: 56154001 | 5 | 56154001 | 56155000 | 1000 | 1 | 2.30E−06 | 0.5844628 | 17 | 1.7 | ANKRD55; RNA5SP184 | |
| DMR5: 57379001 | 5 | 57379001 | 57380000 | 1000 | 1 | 1.46E−07 | −0.5826456 | 10 | 1 | | |
| DMR5: 626750017 | 5 | 62675001 | 62676000 | 1000 | 1 | 4.01E−10 | −0.5902725 | 6 | 0.6 | | |
| DMR5: 65984001 | 5 | 65984001 | 65985000 | 1000 | 1 | 3.74E−06 | −0.5831688 | 12 | 1.2 | ERBIN | |
| DMR5: 75179001 | 5 | 75179001 | 75180000 | 1000 | 1 | 2.07E−06 | −0.5246361 | 6 | 0.6 | ANKRD31 | |
| DMR5: 79263001 | 5 | 79263001 | 79264000 | 1000 | 1 | 1.58E−06 | 0.5879923 | 18 | 1.8 | JMY | |
| DMR5: 80306001 | 5 | 80306001 | 80308000 | 2000 | 1 | 3.68E−07 | 0.6320542 | 34 | 1.7 | AC026410.1; RF00322; AC026410.2 | |
| DMR5: 87198001 | 5 | 87198001 | 87199000 | 1000 | 1 | 9.41E−06 | 0.4671994 | 12 | 1.2 | LINC01949 | |
| DMR5: 94045001 | 5 | 94045001 | 94046000 | 1000 | 1 | 4.21E−06 | 0.8850949 | 3 | 0.3 | FAM172A | Unknown |
| DMR5: 102044001 | 5 | 102044001 | 102045000 | 1000 | 1 | 1.42E−06 | −0.4686804 | 8 | 0.8 | | |
| DMR5: 105574001 | 5 | 105574001 | 105575000 | 1000 | 1 | 9.89E−06 | 0.7868771 | 12 | 1.2 | | |
| DMR5: 105598001 | 5 | 105598001 | 105599000 | 1000 | 1 | 7.36E−06 | −0.6168524 | 7 | 0.7 | | |
| DMR5: 122279001 | 5 | 122279001 | 122280000 | 1000 | 1 | 9.77E−06 | −0.8489853 | 3 | 0.3 | | |
| DMR5: 126188001 | 5 | 126188001 | 126189000 | 1000 | 1 | 1.50E−08 | −1.0559611 | 8 | 0.8 | AC116362.1; LINC02039 | |
| DMR5: 126715001 | 5 | 126715001 | 126716000 | 1000 | 1 | 3.10E−06 | 0.5800275 | 7 | 0.7 | | |
| DMR5: 133134001 | 5 | 133134001 | 133136000 | 2000 | 1 | 8.76E−06 | −0.55855 | 10 | 0.5 | | |
| DMR5: 134757001 | 5 | 134757001 | 134758000 | 1000 | 1 | 6.46E−09 | 1.082406 | 12 | 1.2 | CAMLG; DDX46 | Transcription |
| DMR5: 135090001 | 5 | 135090001 | 135091000 | 1000 | 1 | 7.16E−06 | −0.6150929 | 3 | 0.3 | C5orf66 | |
| DMR5: 135780001 | 5 | 135780001 | 135781000 | 1000 | 1 | 5.96E−06 | 1.1879249 | 25 | 2.5 | SLC25A48 | |
| DMR5: 138562001 | 5 | 138562001 | 138564000 | 2000 | 1 | 1.95E−06 | 0.5819006 | 26 | 1.3 | HSPA9; SNORD63B; SNORD63 | Signaling |
| DMR5: 139469001 | 5 | 139469001 | 139470000 | 1000 | 1 | 2.22E−06 | −0.5321188 | 10 | 1 | AC142391.1; ECSCR; SMIM33; | |
| | | | | | | | | | | TMEM173 | |
| DMR5: 139858001 | 5 | 139858001 | 139860000 | 2000 | 1 | 1.17E−06 | 0.4361855 | 21 | 1.05 | NRG2; AC008667.2 | Signaling |
| DMR5: 141114001 | 5 | 141114001 | 141115000 | 1000 | 1 | 4.39E−07 | −0.4926117 | 6 | 0.6 | AC244517.2; AC244517.1; PCDHB4 | Cytoskeleton |
| DMR5: 160271001 | 5 | 160271001 | 160272000 | 1000 | 1 | 1.26E−06 | −0.533452 | 19 | 1.9 | CCNJL | Signaling |
| DMR5: 164170001 | 5 | 164170001 | 164171000 | 1000 | 1 | 4.13E−06 | −0.6355043 | 8 | 0.8 | AC008662.1 | |
| DMR5: 165244001 | 5 | 165244001 | 165245000 | 1000 | 1 | 2.19E−06 | −0.5904441 | 6 | 0.6 | LINC01938 | |
| DMR5: 173415001 | 5 | 173415001 | 173417000 | 2000 | 1 | 3.69E−06 | −0.427507 | 15 | 0.75 | AC016573.1 | |
| DMR5: 173729001 | 5 | 173729001 | 173730000 | 1000 | 1 | 3.40E−06 | −0.8118145 | 12 | 1.2 | LINC01484 | |
| DMR5: 175062001 | 5 | 175062001 | 175063000 | 1000 | 1 | 6.94E−07 | −0.4382482 | 11 | 1.1 | | |
| DMR5: 177954001 | 5 | 177954001 | 177955000 | 1000 | 1 | 8.84E−06 | 0.5201743 | 14 | 1.4 | AC106795.1; AC106795.3; AC106795.2 | |
| DMR5: 179132001 | 5 | 179132001 | 179138000 | 6000 | 1 | 8.10E−06 | 0.9730536 | 168 | 2.8 | ADAMTS2 | Protease |
| DMR5: 179969001 | 5 | 179969001 | 179970000 | 1000 | 1 | 5.15E−06 | 0.6155863 | 15 | 1.5 | RNF130; AC010285.1; AC010285.3 | Metabolism |
| DMR5: 180779001 | 5 | 180779001 | 180780000 | 1000 | 1 | 3.90E−06 | −0.4453688 | 12 | 1.2 | MGAT1 | Metabolism |
| DMR6: 1402001 | 6 | 1402001 | 1404000 | 2000 | 2 | 2.66E−06 | −0.4116056 | 34 | 1.7 | FOXF2 | Transcription |
| DMR6: 3924001 | 6 | 3924001 | 3925000 | 1000 | 1 | 3.73E−06 | −0.5157897 | 13 | 1.3 | AL590004.2; AL590004.1 | |
| DMR6: 6430001 | 6 | 6430001 | 6431000 | 1000 | 1 | 9.25E−06 | −0.5263292 | 5 | 0.5 | LY86-AS1 | |
| DMR6: 6718001 | 6 | 6718001 | 6721000 | 3000 | 1 | 1.16E−06 | −0.8640551 | 34 | 1.133 | AL031123.1 | |
| DMR6: 11738001 | 6 | 11738001 | 11741000 | 3000 | 1 | 6.40E−06 | −0.7211799 | 27 | 0.9 | ADTRP | Development |
| DMR6: 12921001 | 6 | 12921001 | 12922000 | 1000 | 1 | 1.52E−06 | 0.9800264 | 2 | 0.2 | PHACTR1 | Signaling |
| DMR6: 13133001 | 6 | 13133001 | 13134000 | 1000 | 1 | 7.27E−06 | 0.602249 | 51 | 5.1 | PHACTR1 | Signaling |
| DMR6: 14622001 | 6 | 14622001 | 14623000 | 1000 | 1 | 5.64E−06 | −0.4587547 | 22 | 2.2 | | |
| DMR6: 19698001 | 6 | 19698001 | 19699000 | 1000 | 1 | 6.34E−07 | −0.3771312 | 17 | 1.7 | AL022068.1 | |
| DMR6: 21055001 | 6 | 21055001 | 21056000 | 1000 | 1 | 2.87E−06 | −1.1051213 | 3 | 0.3 | CDKAL1 | Cell Cycle |
| DMR6: 24039001 | 6 | 24039001 | 24041000 | 2000 | 1 | 8.51E−06 | −0.9233759 | 7 | 0.35 | | |
| DMR6: 28926001 | 6 | 28926001 | 28927000 | 1000 | 1 | 8.45E−06 | 0.8705091 | 15 | 1.5 | TRIM27 | Metabolism |
| DMR6: 29618001 | 6 | 29618001 | 29619000 | 1000 | 1 | 1.19E−06 | −0.636553 | 6 | 0.6 | GABBR1 | Receptor |
| DMR6: 38496001 | 6 | 38496001 | 38497000 | 1000 | 1 | 1.42E−06 | −0.7941924 | 7 | 0.7 | BTBD9 | Unknown |
| DMR6: 38624001 | 6 | 38624001 | 38625000 | 1000 | 1 | 2.89E−07 | −0.6006061 | 15 | 1.5 | BTBD9 | Unknown |
| DMR6: 43546001 | 6 | 43546001 | 43547000 | 1000 | 1 | 6.01E−06 | −1.0896769 | 5 | 0.5 | POLR1C; XPO5; AL355802.1; RF00426 | Translation; Receptor |
| DMR6: 43856001 | 6 | 43856001 | 43857000 | 1000 | 1 | 4.81E−07 | −1.1220187 | 6 | 0.6 | LINC02537; AL157371.1 | |
| DMR6: 49550001 | 6 | 49550001 | 49551000 | 1000 | 1 | 9.52E−07 | 0.8714945 | 49 | 4.9 | C6orf141 | |
| DMR6: 52624001 | 6 | 52624001 | 52625000 | 1000 | 1 | 6.99E−06 | 0.7442294 | 16 | 1.6 | | |
| DMR6: 79220001 | 6 | 79220001 | 79221000 | 1000 | 1 | 4.37E−06 | 0.6615165 | 9 | 0.9 | HMGN3 | Epigenetic |
| DMR6: 89472001 | 6 | 89472001 | 89473000 | 1000 | 1 | 7.35E−06 | −0.7215289 | 11 | 1.1 | ANKRD6; RN7SL11P | |
| DMR6: 91335001 | 6 | 91335001 | 91336000 | 1000 | 1 | 4.31E−06 | −0.5591004 | 7 | 0.7 | | |
| DMR6: 97192001 | 6 | 97192001 | 97193000 | 1000 | 1 | 4.66E−06 | 1.016525 | 3 | 0.3 | MMS22L | |
| DMR6: 99166001 | 6 | 99166001 | 99168000 | 2000 | 1 | 9.50E−07 | −0.6259245 | 9 | 0.45 | BDH2P1 | |
| DMR6: 99861001 | 6 | 99861001 | 99862000 | 1000 | 1 | 8.83E−09 | −1.0353924 | 3 | 0.3 | | |
| DMR6: 102673001 | 6 | 102673001 | 102675000 | 2000 | 1 | 9.49E−08 | −0.6446693 | 12 | 0.6 | | |
| DMR6: 104679001 | 6 | 104679001 | 104680000 | 1000 | 1 | 2.88E−06 | 0.7593947 | 6 | 0.6 | AL356967.1 | |
| DMR6: 110799001 | 6 | 110799001 | 110801000 | 2000 | 1 | 1.52E−06 | −0.6955559 | 14 | 0.7 | CDK19 | Signaling |
| DMR6: 119104001 | 6 | 119104001 | 119105000 | 1000 | 1 | 1.35E−06 | 1.016355 | 8 | 0.8 | AL137009.1; FAM184A | Unknown |
| DMR6: 119412001 | 6 | 119412001 | 119414000 | 2000 | 1 | 6.30E−06 | −0.7922276 | 6 | 0.3 | | |
| DMR6: 119589001 | 6 | 119589001 | 119590000 | 1000 | 1 | 3.17E−06 | −0.4975542 | 3 | 0.3 | | |
| DMR6: 122964001 | 6 | 122964001 | 122965000 | 1000 | 1 | 8.16E−06 | −1.0593466 | 1 | 0.1 | | |
| DMR6: 124759001 | 6 | 124759001 | 124761000 | 2000 | 1 | 5.75E−07 | 0.6666712 | 21 | 1.05 | NKAIN2 | Transport |
| DMR6: 135329001 | 6 | 135329001 | 135332000 | 3000 | 1 | 3.71E−06 | −0.869098 | 25 | 0.833 | AHI1 | Development |
| DMR6: 150690001 | 6 | 150690001 | 150691000 | 1000 | 1 | 9.60E−06 | −0.5202252 | 10 | 1 | PLEKHG1 | Signaling |
| DMR6: 151058001 | 6 | 151058001 | 151059000 | 1000 | 1 | 2.29E−06 | 0.6011066 | 16 | 1.6 | MTHFD1L; AL133260.2 | Metabolism |
| DMR6: 154006001 | 6 | 154006001 | 154007000 | 1000 | 1 | 3.02E−06 | −0.7391696 | 3 | 0.3 | OPRM1 | Receptor |
| DMR6: 156358001 | 6 | 156358001 | 156360000 | 2000 | 1 | 3.95E−06 | −0.435247 | 27 | 1.35 | AL512658.2 | |
| DMR6: 159658001 | 6 | 159658001 | 159659000 | 1000 | 1 | 4.31E−07 | −0.510362 | 5 | 0.5 | | |
| DMR6: 159707001 | 6 | 159707001 | 159709000 | 2000 | 1 | 2.36E−07 | −0.6397778 | 13 | 0.65 | SOD2; HNRNPH1P1 | Metabolism |
| DMR6: 160945001 | 6 | 160945001 | 160948000 | 3000 | 1 | 2.05E−06 | −0.4429917 | 33 | 1.1 | AL139393.1 | |
| DMR6: 167422001 | 6 | 167422001 | 167424000 | 2000 | 1 | 5.02E−07 | 0.7600747 | 143 | 7.15 | | |
| DMR7: 1273001 | 7 | 1273001 | 1279000 | 6000 | 1 | 5.10E−06 | 0.5570609 | 200 | 3.333 | AC073094.1 | |
| DMR7: 2902001 | 7 | 2902001 | 2904000 | 2000 | 1 | 1.27E−07 | 0.7713864 | 35 | 1.75 | CARD11 | Unknown |
| DMR7: 7648001 | 7 | 7648001 | 7650000 | 2000 | 1 | 5.29E−07 | −0.6083148 | 20 | 1 | RPA3; UMAD1; AC007161.3 | Cytoskeleton |
| DMR7: 17916001 | 7 | 17916001 | 17918000 | 2000 | 1 | 1.28E−06 | −0.6276699 | 13 | 0.65 | SNX13 | Signaling |
| DMR7: 26081001 | 7 | 26081001 | 26083000 | 2000 | 1 | 1.79E−06 | −0.7509067 | 14 | 0.7 | | |
| DMR7: 28416001 | 7 | 28416001 | 28418000 | 2000 | 1 | 4.34E−06 | −0.53156 | 16 | 0.8 | CREB5 | Transcription |
| DMR7: 30512001 | 7 | 30512001 | 30514000 | 2000 | 1 | 8.02E−06 | −0.4580246 | 20 | 1 | GGCT; AC005154.5; GARS-DT; | |
| | | | | | | | | | | AC005154.2 | |
| DMR7: 32601001 | 7 | 32601001 | 32602000 | 1000 | 1 | 1.07E−06 | −0.6311666 | 8 | 0.8 | DPY19L1P1 | |
| DMR7: 35347001 | 7 | 35347001 | 35348000 | 1000 | 1 | 6.54E−06 | 0.8059713 | 12 | 1.2 | | |
| DMR7: 35750001 | 7 | 35750001 | 35751000 | 1000 | 1 | 3.61E−06 | −0.456453 | 5 | 0.5 | SEPT7-AS1 | |
| DMR7: 37123001 | 7 | 37123001 | 37124000 | 1000 | 1 | 2.27E−07 | −0.5588348 | 5 | 0.5 | ELMO1; RPS17P13 | Signaling |
| DMR7: 37689001 | 7 | 37689001 | 37691000 | 2000 | 1 | 9.39E−06 | −0.4653141 | 19 | 0.95 | GPR141; EPDR1 | Receptor |
| DMR7: 38672001 | 7 | 38672001 | 38673000 | 1000 | 1 | 8.17E−06 | −0.5160182 | 12 | 1.2 | | |
| DMR7: 40481001 | 7 | 40481001 | 40482000 | 1000 | 1 | 4.84E−07 | 1.0367547 | 9 | 0.9 | SUGCT | EST |
| DMR7: 42733001 | 7 | 42733001 | 42734000 | 1000 | 1 | 5.91E−09 | −0.6650931 | 10 | 1 | | |
| DMR7: 42927001 | 7 | 42927001 | 42929000 | 2000 | 1 | 6.42E−06 | 0.7891598 | 19 | 0.95 | AC010132.3; PSMA2; AC010132.2; | |
| | | | | | | | | | | MRPL32 | |
| DMR7: 44024001 | 7 | 44024001 | 44026000 | 2000 | 1 | 3.41E−06 | 0.7581505 | 18 | 0.9 | POLR2J4; AC004951.4; AC017116.2; | |
| | | | | | | | | | | RASA4CP | |
| DMR7: 44804001 | 7 | 44804001 | 44805000 | 1000 | 1 | 7.50E−06 | 0.6919024 | 13 | 1.3 | PPIA | |
| DMR7: 46116001 | 7 | 46116001 | 46117000 | 1000 | 1 | 1.39E−06 | −0.5695176 | 8 | 0.8 | | |
| DMR7: 50060001 | 7 | 50060001 | 50061000 | 1000 | 1 | 2.21E−06 | −0.6013637 | 5 | 0.5 | ZP BP | Development |
| DMR7: 50814001 | 7 | 50814001 | 50815000 | 1000 | 1 | 5.01E−06 | 0.5820185 | 11 | 1.1 | | |
| DMR7: 53275001 | 7 | 53275001 | 53276000 | 1000 | 1 | 2.40E−06 | 0.6137477 | 9 | 0.9 | | |
| DMR7: 65521001 | 7 | 65521001 | 65522000 | 1000 | 1 | 6.72E−06 | −0.8485457 | 9 | 0.9 | AC114501.2 | |
| DMR7: 68057001 | 7 | 68057001 | 68058000 | 1000 | 1 | 8.28E−06 | 0.7511854 | 9 | 0.9 | | |
| DMR7: 72788001 | 7 | 72788001 | 72791000 | 3000 | 1 | 9.28E−06 | 0.5741053 | 42 | 1.4 | TYW1B | |
| DMR7: 73698001 | 7 | 73698001 | 73699000 | 1000 | 1 | 3.21E−06 | 0.5988557 | 12 | 1.2 | BUD23; STX1A | Metabolism; Transport |
| DMR7: 74011001 | 7 | 74011001 | 74012000 | 1000 | 1 | 1.47E−06 | 0.7238667 | 18 | 1.8 | | |
| DMR7: 74096001 | 7 | 74096001 | 74097000 | 1000 | 1 | 5.46E−06 | 0.5571859 | 22 | 2.2 | LIMK1 | Signaling |
| DMR7: 98826001 | 7 | 98826001 | 98827000 | 1000 | 1 | 7.36E−06 | −0.4244376 | 31 | 3.1 | | |
| DMR7: 103693001 | 7 | 103693001 | 103695000 | 2000 | 1 | 6.89E−06 | −0.7201378 | 12 | 0.6 | RELN | Protease |
| DMR7: 110708001 | 7 | 110708001 | 110712000 | 4000 | 1 | 4.02E−08 | −0.7197439 | 41 | 1.025 | IMMP2L | Protease |
| DMR7: 110843001 | 7 | 110843001 | 110845000 | 2000 | 1 | 6.88E−07 | −0.7812022 | 15 | 0.75 | IMMP2L | Protease |
| DMR7: 130079001 | 7 | 130079001 | 130080000 | 1000 | 1 | 3.97E−06 | 0.5701097 | 13 | 1.3 | KLHDC10 | |
| DMR7: 131148001 | 7 | 131148001 | 131150000 | 2000 | 1 | 3.71E−07 | −0.6301175 | 19 | 0.95 | MKLN1 | Signaling |
| DMR7: 133296001 | 7 | 133296001 | 133297000 | 1000 | 1 | 4.30E−06 | −0.815133 | 3 | 0.3 | EXOC4; MIR6133 | Transport |
| DMR7: 142062001 | 7 | 142062001 | 142063000 | 1000 | 1 | 5.33E−07 | −0.8628877 | 12 | 1.2 | MGAM | Metabolism |
| DMR7: 142930001 | 7 | 142930001 | 142931000 | 1000 | 1 | 2.48E−06 | −0.547 | 15 | 1.5 | TRPV5; LLCFC1 | Transport |
| DMR7: 146096001 | 7 | 146096001 | 146097000 | 1000 | 1 | 2.61E−06 | 0.8492238 | 39 | 3.9 | | |
| DMR7: 157874001 | 7 | 157874001 | 157875000 | 1000 | 1 | 1.39E−06 | −0.3499685 | 27 | 2.7 | PTPRN2; AC011899.3 | Signaling |
| DMR8: 482001 | 8 | 482001 | 483000 | 1000 | 1 | 4.98E−07 | −0.6810516 | 17 | 1.7 | FBXO25; AC083964.2; TDRP | |
| DMR8: 1242001 | 8 | 1242001 | 1244000 | 2000 | 1 | 1.88E−07 | −0.4707801 | 44 | 2.2 | DLGAP2; AC110288.1 | Receptor |
| DMR8: 1824001 | 8 | 1824001 | 1826000 | 2000 | 2 | 5.56E−10 | 1.0155084 | 96 | 4.8 | AC019257.8; MIR596; ARHGEF10 | Protease |
| DMR8: 5650001 | 8 | 5650001 | 5654000 | 4000 | 1 | 1.64E−07 | −0.5336835 | 46 | 1.15 | AC084768.1 | |
| DMR8: 6559001 | 8 | 6559001 | 6560000 | 1000 | 1 | 1.10E−06 | 0.8944343 | 14 | 1.4 | MCPH1; ANGPT2 | DNA Repair; Signaling |
| DMR8: 7690001 | 8 | 7690001 | 7691000 | 1000 | 1 | 2.61E−07 | 1.0332821 | 14 | 1.4 | AC084121.3; AC084121.4; AC084121.2 | |
| DMR8: 10430001 | 8 | 10430001 | 10432000 | 2000 | 1 | 2.86E−06 | −0.4179805 | 33 | 1.65 | MSRA; AC104964.3 | Metabolism |
| DMR8: 12623001 | 8 | 12623001 | 12625000 | 2000 | 1 | 2.45E−07 | −1.1402102 | 29 | 1.45 | AC068587.4; RPS3AP35 | |
| DMR8: 18621001 | 8 | 18621001 | 18623000 | 2000 | 1 | 5.96E−06 | 0.6731904 | 20 | 1 | PSD3 | Signaling |
| DMR8: 22162001 | 8 | 22162001 | 22164000 | 2000 | 1 | 1.60E−06 | −0.5803564 | 39 | 1.95 | LGI3; SFTPC; BMP1; AC105206.1 | Receptor; Unknown; Protease |
| DMR8: 25681001 | 8 | 25681001 | 25683000 | 2000 | 1 | 1.05E−06 | −0.4786134 | 10 | 0.5 | AC009623.1 | |
| DMR8: 26563001 | 8 | 26563001 | 26565000 | 2000 | 1 | 8.10E−07 | −0.3632575 | 14 | 0.7 | DPYSL2 | Metabolism |
| DMR8: 35097001 | 8 | 35097001 | 35098000 | 1000 | 1 | 7.68E−06 | −0.9679379 | 6 | 0.6 | | |
| DMR8: 38869001 | 8 | 38869001 | 38870000 | 1000 | 1 | 3.10E−06 | 1.0914025 | 9 | 0.9 | | |
| DMR8: 48530001 | 8 | 48530001 | 48531000 | 1000 | 1 | 2.53E−06 | −0.9712279 | 6 | 0.6 | | |
| DMR8: 51871001 | 8 | 51871001 | 51872000 | 1000 | 1 | 8.41E−06 | −0.5646483 | 6 | 0.6 | PCMTD1 | Metabolism |
| DMR8: 53301001 | 8 | 53301001 | 53302000 | 1000 | 1 | 8.87E−06 | −0.8694708 | 5 | 0.5 | | |
| DMR8: 59382001 | 8 | 59382001 | 59383000 | 1000 | 1 | 9.68E−08 | −0.6404769 | 11 | 1.1 | | |
| DMR8: 67401001 | 8 | 67401001 | 67402000 | 1000 | 1 | 2.11E−06 | 0.7281808 | 7 | 0.7 | AC011037.1 | |
| DMR8: 71115001 | 8 | 71115001 | 71116000 | 1000 | 1 | 9.76E−06 | 0.8768569 | 13 | 1.3 | AC015687.1 | |
| DMR8: 76198001 | 8 | 76198001 | 76199000 | 1000 | 1 | 3.52E−08 | −0.6295675 | 5 | 0.5 | | |
| DMR8: 81328001 | 8 | 81328001 | 81329000 | 1000 | 1 | 6.17E−07 | −0.4740234 | 6 | 0.6 | | |
| DMR8: 84728001 | 8 | 84728001 | 84730000 | 2000 | 1 | 1.43E−06 | −0.5480921 | 10 | 0.5 | RALYL | Transcription |
| DMR8: 85216001 | 8 | 85216001 | 85217000 | 1000 | 1 | 1.62E−06 | −0.5208883 | 7 | 0.7 | E2F5; C8orf59; CA13; AC011773.3 | Transcription; Metabolism |
| DMR8: 86444001 | 8 | 86444001 | 86446000 | 2000 | 1 | 4.72E−07 | −0.8015767 | 9 | 0.45 | WWP1 | Proteolysis |
| DMR8: 88448001 | 8 | 88448001 | 88449000 | 1000 | 1 | 1.24E−06 | 0.8389684 | 10 | 1 | AC090578.1 | |
| DMR8: 88646001 | 8 | 88646001 | 88647000 | 1000 | 1 | 9.72E−08 | −0.8520037 | 3 | 0.3 | AC090578.1 | |
| DMR8: 91548001 | 8 | 91548001 | 91550000 | 2000 | 1 | 1.28E−07 | −1.1503793 | 21 | 1.05 | AC103409.1 | |
| DMR8: 95873001 | 8 | 95873001 | 95875000 | 2000 | 1 | 8.32E−06 | −0.6286364 | 8 | 0.4 | | |
| DMR8: 96325001 | 8 | 96325001 | 96326000 | 1000 | 1 | 2.37E−09 | −0.6699008 | 6 | 0.6 | PTDSS1 | Metabolism |
| DMR8: 97331001 | 8 | 97331001 | 97332000 | 1000 | 1 | 9.10E−06 | −0.5777146 | 10 | 1 | | |
| DMR8: 114583001 | 8 | 114583001 | 114585000 | 2000 | 1 | 3.44E−07 | −0.5416655 | 17 | 0.85 | | |
| DMR8: 118551001 | 8 | 118551001 | 118553000 | 2000 | 1 | 6.80E−07 | −0.5612178 | 17 | 0.85 | SAMD12 | Unknown |
| DMR8: 118685001 | 8 | 118685001 | 118687000 | 2000 | 1 | 3.20E−07 | −0.6557276 | 15 | 0.75 | SAMD12-AS1 | |
| DMR8: 120190001 | 8 | 120190001 | 120192000 | 2000 | 1 | 8.49E−06 | −0.5971808 | 10 | 0.5 | COL14A1 | Cytoskeleton |
| DMR8: 122964001 | 8 | 122964001 | 122966000 | 2000 | 1 | 1.56E−06 | −0.7544156 | 9 | 0.45 | ZHX2 | Transcription |
| DMR8: 124750001 | 8 | 124750001 | 124751000 | 1000 | 1 | 5.93E−06 | 0.7510927 | 14 | 1.4 | | |
| DMR8: 125639001 | 8 | 125639001 | 125641000 | 2000 | 1 | 7.56E−07 | −0.5941291 | 27 | 1.35 | AC016074.2 | |
| DMR8: 127990001 | 8 | 127990001 | 127991000 | 1000 | 1 | 3.65E−06 | −0.5753967 | 7 | 0.7 | PVT1; RNU1-106P | |
| DMR8: 133091001 | 8 | 133091001 | 133093000 | 2000 | 1 | 1.34E−07 | −0.4136718 | 30 | 1.5 | TG; SLA | Signaling |
| DMR8: 133250001 | 8 | 133250001 | 133251000 | 1000 | 1 | 6.01E−06 | −0.6032632 | 18 | 1.8 | NDRG1 | Transcription |
| DMR8: 136037001 | 8 | 136037001 | 136039000 | 2000 | 1 | 2.28E−07 | 1.0371973 | 23 | 1.15 | LINC02055 | |
| DMR8: 142072001 | 8 | 142072001 | 142073000 | 1000 | 1 | 6.37E−07 | 0.8235722 | 13 | 1.3 | | |
| DMR9: 24711001 | 9 | 24711001 | 24712000 | 1000 | 1 | 1.13E−06 | −1.0081227 | 3 | 0.3 | | |
| DMR9: 25308001 | 9 | 25308001 | 25310000 | 2000 | 1 | 5.33E−07 | 0.8622406 | 19 | 0.95 | | |
| DMR9: 27371001 | 9 | 27371001 | 27372000 | 1000 | 1 | 6.39E−06 | −0.4952696 | 11 | 1.1 | MOB3B | Signaling |
| DMR9: 31375001 | 9 | 31375001 | 31376000 | 1000 | 1 | 3.62E−08 | −0.4665531 | 10 | 1 | LINC01243 | |
| DMR9: 62708001 | 9 | 62708001 | 62710000 | 2000 | 1 | 4.99E−06 | 0.7495045 | 39 | 1.95 | FKBP4P7 | |
| DMR9: 65560001 | 9 | 65560001 | 65561000 | 1000 | 1 | 7.15E−06 | −0.6755017 | 7 | 0.7 | | |
| DMR9: 72375001 | 9 | 72375001 | 72377000 | 2000 | 1 | 5.56E−07 | 0.6838577 | 33 | 1.65 | ZFAND5 | Transcription |
| DMR9: 82141001 | 9 | 82141001 | 82142000 | 1000 | 1 | 3.98E−06 | 0.8524656 | 32 | 3.2 | AL162726.3; AL158047.1 | |
| DMR9: 87814001 | 9 | 87814001 | 87816000 | 2000 | 1 | 1.99E−07 | −0.9849743 | 9 | 0.45 | AL772337.4; FBP2P1; ELF2P3; NPAP1P7 | |
| DMR9: 88286001 | 9 | 88286001 | 88287000 | 1000 | 1 | 3.02E−08 | 0.6938888 | 18 | 1.8 | | |
| DMR9: 90190001 | 9 | 90190001 | 90192000 | 2000 | 1 | 9.63E−06 | −1.0380804 | 7 | 0.35 | | |
| DMR9: 90411001 | 9 | 90411001 | 90413000 | 2000 | 1 | 6.89E−06 | −0.5865592 | 13 | 0.65 | LINC01508 | |
| DMR9: 94745001 | 9 | 94745001 | 94747000 | 2000 | 1 | 3.85E−06 | −0.5994547 | 27 | 1.35 | C9orf3 | Proteolysis |
| DMR9: 98603001 | 9 | 98603001 | 98606000 | 3000 | 1 | 6.91E−06 | −0.7573327 | 44 | 1.467 | GABBR2; SEPT7P7 | Receptor |
| DMR9: 100217001 | 9 | 100217001 | 100218000 | 1000 | 1 | 9.55E−06 | 0.8744344 | 11 | 1.1 | INVS | |
| DMR9: 102024001 | 9 | 102024001 | 102027000 | 3000 | 1 | 6.18E−06 | −0.8691895 | 11 | 0.367 | | |
| DMR9: 116117001 | 9 | 116117001 | 116118000 | 1000 | 1 | 8.04E−07 | −0.6406795 | 7 | 0.7 | | |
| DMR9: 121919001 | 9 | 121919001 | 121920000 | 1000 | 1 | 9.38E−06 | −0.6209179 | 4 | 0.4 | TTLL11; TTLL11-IT1 | Cytoskeleton |
| DMR9: 124096001 | 9 | 124096001 | 124097000 | 1000 | 1 | 2.94E−06 | 0.5522826 | 18 | 1.8 | | |
| DMR9: 125187001 | 9 | 125187001 | 125189000 | 2000 | 1 | 1.44E−06 | 0.8385809 | 58 | 2.9 | PPP6C; RPSAP76 | Signaling |
| DMR9: 128010001 | 9 | 128010001 | 128012000 | 2000 | 1 | 9.26E−06 | 0.532175 | 31 | 1.55 | | |
| DMR9: 128486001 | 9 | 128486001 | 128487000 | 1000 | 1 | 2.59E−06 | −1.0404849 | 3 | 0.3 | ODF2 | Cytoskeleton |
| DMR9: 129113001 | 9 | 129113001 | 129114000 | 1000 | 1 | 3.34E−07 | 0.587374 | 23 | 2.3 | CRAT; PTPA | Metabolism |
| DMR9: 134443001 | 9 | 134443001 | 134446000 | 3000 | 1 | 9.49E−07 | 0.9415373 | 53 | 1.767 | RXRA | Receptor |
| DMR9: 137140001 | 9 | 137140001 | 137142000 | 2000 | 1 | 1.23E−06 | −0.7865738 | 51 | 2.55 | GRIN1 | Receptor |
| DMR10: 5881001 | 10 | 5881001 | 5883000 | 2000 | 1 | 1.72E−06 | 0.645441 | 33 | 1.65 | ANKRD16; FBH1 | |
| DMR10: 6852001 | 10 | 6852001 | 6853000 | 1000 | 1 | 7.94E−06 | 1.0386449 | 9 | 0.9 | LINC00707; AL392086.2 | |
| DMR10: 6930001 | 10 | 6930001 | 6931000 | 1000 | 1 | 6.57E−06 | −0.8284854 | 4 | 0.4 | AL392086.1 | |
| DMR10: 7527001 | 10 | 7527001 | 7528000 | 1000 | 1 | 3.74E−06 | −0.4612989 | 7 | 0.7 | AL445070.1 | |
| DMR10: 19091001 | 10 | 19091001 | 19092000 | 1000 | 1 | 1.35E−07 | −0.5344647 | 8 | 0.8 | MALRD1 | |
| DMR10: 20290001 | 10 | 20290001 | 20292000 | 2000 | 1 | 2.52E−07 | 0.7460668 | 15 | 0.75 | PLXDC2 | Binding Protein |
| DMR10: 20821001 | 10 | 20821001 | 20822000 | 1000 | 1 | 1.28E−09 | −0.7634124 | 6 | 0.6 | NEBL | Cytoskeleton |
| DMR10: 26085001 | 10 | 26085001 | 26087000 | 2000 | 1 | 7.85E−06 | −0.7895772 | 9 | 0.45 | MYO3A | Cytoskeleton |
| DMR10: 27491001 | 10 | 27491001 | 27492000 | 1000 | 1 | 1.51E−08 | −0.6822888 | 7 | 0.7 | | |
| DMR10: 27628001 | 10 | 27628001 | 27629000 | 1000 | 1 | 3.66E−06 | −0.6216715 | 13 | 1.3 | | |
| DMR10: 34836001 | 10 | 34836001 | 34837000 | 1000 | 1 | 7.09E−06 | −0.3568398 | 15 | 1.5 | | |
| DMR10: 37507001 | 10 | 37507001 | 37508000 | 1000 | 1 | 3.45E−06 | 1.0486368 | 16 | 1.6 | | |
| DMR10: 43225001 | 10 | 43225001 | 43226000 | 1000 | 1 | 1.77E−06 | −0.3470552 | 12 | 1.2 | RASGEF1A | Signaling |
| DMR10: 49630001 | 10 | 49630001 | 49633000 | 3000 | 1 | 2.75E−06 | −0.3770004 | 37 | 1.233 | CHAT | Metabolism |
| DMR10: 49806001 | 10 | 49806001 | 49807000 | 1000 | 1 | 1.26E−06 | 0.7055622 | 18 | 1.8 | RPL21P89 | |
| DMR10: 54518001 | 10 | 54518001 | 54519000 | 1000 | 1 | 9.94E−06 | −0.4557692 | 9 | 0.9 | PCDH15; AL353784.1 | Extracellular Matrix |
| DMR10: 57997001 | 10 | 57997001 | 57998000 | 1000 | 1 | 2.56E−06 | −0.5250618 | 6 | 0.6 | | |
| DMR10: 64949001 | 10 | 64949001 | 64951000 | 2000 | 1 | 9.17E−07 | −0.5648882 | 6 | 0.3 | | |
| DMR10: 69258001 | 10 | 69258001 | 69260000 | 2000 | 1 | 7.13E−07 | −0.5566237 | 14 | 0.7 | HKDC1; AL596223.2; HK1 | Signaling |
| DMR10: 70813001 | 10 | 70813001 | 70814000 | 1000 | 1 | 6.98E−06 | −0.4802403 | 11 | 1.1 | SGPL1 | Metabolism |
| DMR10: 72197001 | 10 | 72197001 | 72199000 | 2000 | 1 | 2.65E−06 | 0.5785468 | 22 | 1.1 | ASCC1; RPL15P14 | Binding Protein |
| DMR10: 73625001 | 10 | 73625001 | 73626000 | 1000 | 1 | 1.86E−06 | 0.7193911 | 55 | 5.5 | USP54; AC073389.1; AC073389.3; | |
| | | | | | | | | | | MYOZ1 | |
| DMR10: 76218001 | 10 | 76218001 | 76222000 | 4000 | 1 | 5.21E−06 | −0.653641 | 30 | 0.75 | LRMDA | |
| DMR10: 77091001 | 10 | 77091001 | 77092000 | 1000 | 1 | 9.93E−07 | −0.6685994 | 14 | 1.4 | KCNMA1 | Metabolism |
| DMR10: 79033001 | 10 | 79033001 | 79035000 | 2000 | 1 | 1.90E−06 | −0.2902581 | 32 | 1.6 | ZMIZ1-AS1 | |
| DMR10: 79325001 | 10 | 79325001 | 79328000 | 3000 | 1 | 5.79E−06 | −0.3867976 | 21 | 0.7 | ZMIZ1 | Metabolism |
| DMR10: 86365001 | 10 | 86365001 | 86366000 | 1000 | 1 | 4.36E−07 | 0.767855 | 62 | 6.2 | GRID1 | Receptor |
| DMR10: 91469001 | 10 | 91469001 | 91472000 | 3000 | 1 | 7.56E−06 | −0.8092263 | 13 | 0.433 | HECTD2; AL161798.1 | EST |
| DMR10: 100809001 | 10 | 100809001 | 100810000 | 1000 | 1 | 2.62E−06 | 0.6844549 | 15 | 1.5 | PAX2 | Transcription |
| DMR10: 101619001 | 10 | 101619001 | 101622000 | 3000 | 1 | 1.67E−06 | 1.2099467 | 40 | 1.333 | DPCD; FBXW4; RNU6-1165P | Unknown |
| DMR10: 101661001 | 10 | 101661001 | 101663000 | 2000 | 1 | 4.42E−08 | −0.5988523 | 19 | 0.95 | FBXW4 | Unknown |
| DMR10: 103389001 | 10 | 103389001 | 103392000 | 3000 | 1 | 8.61E−06 | 0.6316328 | 44 | 1.467 | TAF5; ATP5MD; MIR1307; PDCD11 | Transcription; Apoptosis |
| DMR10: 106795001 | 10 | 106795001 | 106796000 | 1000 | 1 | 4.54E−06 | −0.6829713 | 7 | 0.7 | SORCS1 | Receptor |
| DMR10: 113356001 | 10 | 113356001 | 113357000 | 1000 | 1 | 6.23E−06 | −0.9072859 | 7 | 0.7 | RNU7-165P | |
| DMR10: 117499001 | 10 | 117499001 | 117501000 | 2000 | 1 | 8.78E−06 | −0.6012907 | 30 | 1.5 | AC005871.2; EMX2OS | |
| DMR10: 118021001 | 10 | 118021001 | 118023000 | 2000 | 1 | 5.68E−08 | −0.4882908 | 13 | 0.65 | RAB11FIP2; AC022395.1 | |
| DMR10: 125943001 | 10 | 125943001 | 125944000 | 1000 | 1 | 4.47E−06 | 1.042737 | 12 | 1.2 | FANK1 | |
| DMR10: 133012001 | 10 | 133012001 | 133013000 | 1000 | 1 | 7.74E−06 | −0.4565435 | 49 | 4.9 | | |
| DMR11: 3911001 | 11 | 3911001 | 3913000 | 2000 | 1 | 3.62E−06 | 0.633939 | 9 | 0.45 | STIM1; RF00409 | Signaling |
| DMR11: 6281001 | 11 | 6281001 | 6282000 | 1000 | 1 | 9.32E−06 | 0.5672051 | 16 | 1.6 | CCKBR | Receptor |
| DMR11: 14276001 | 11 | 14276001 | 14278000 | 2000 | 1 | 5.87E−08 | 0.5808539 | 17 | 0.85 | SPON1; SPON1-AS1; RRAS2 | Growth Factors & Cytokines; |
| | | | | | | | | | | | Signaling |
| DMR11: 19359001 | 11 | 19359001 | 19360000 | 1000 | 1 | 2.69E−08 | −0.6964187 | 8 | 0.8 | NAV2 | Development |
| DMR11: 22614001 | 11 | 22614001 | 22615000 | 1000 | 1 | 5.04E−07 | −0.72362 | 5 | 0.5 | FANCF | |
| DMR11: 24281001 | 11 | 24281001 | 24283000 | 2000 | 1 | 4.83E−06 | −0.5355246 | 17 | 0.85 | | |
| DMR11: 24960001 | 11 | 24960001 | 24962000 | 2000 | 1 | 9.40E−07 | −0.490419 | 11 | 0.55 | LUZP2 | |
| DMR11: 31576001 | 11 | 31576001 | 31578000 | 2000 | 1 | 8.37E−06 | −0.4835536 | 17 | 0.85 | ELP4 | Transcription |
| DMR11: 33571001 | 11 | 33571001 | 33573000 | 2000 | 1 | 4.44E−06 | 0.7680501 | 21 | 1.05 | KIAA1549L | |
| DMR11: 35771001 | 11 | 35771001 | 35772000 | 1000 | 1 | 3.97E−07 | 0.5739109 | 14 | 1.4 | TRIM44 | |
| DMR11: 40588001 | 11 | 40588001 | 40589000 | 1000 | 1 | 8.95E−06 | −0.3503883 | 9 | 0.9 | LRRC4C | Extracellular Matrix |
| DMR11: 46022001 | 11 | 46022001 | 46025000 | 3000 | 1 | 6.14E−08 | 0.7306804 | 24 | 0.8 | PHF21A | Metabolism |
| DMR11: 49397001 | 11 | 49397001 | 49398000 | 1000 | 1 | 5.12E−06 | −0.6419734 | 4 | 0.4 | TYRL; CBX3P8 | |
| DMR11: 56338001 | 11 | 56338001 | 56340000 | 2000 | 1 | 7.61E−06 | −0.816767 | 16 | 0.8 | FAM8A2P; OR8K2P; OR8K1 | Receptor |
| DMR11: 61100001 | 11 | 61100001 | 61101000 | 1000 | 1 | 3.13E−06 | 0.6428472 | 2 | 0.2 | CD5 | Receptor |
| DMR11: 62013001 | 11 | 62013001 | 62014000 | 1000 | 1 | 6.72E−06 | −0.8931881 | 12 | 1.2 | AP003733.1 | |
| DMR11: 63013001 | 11 | 63013001 | 63014000 | 1000 | 1 | 2.77E−06 | −0.6064386 | 10 | 1 | SLC22A8 | Transport |
| DMR11: 71288001 | 11 | 71288001 | 71289000 | 1000 | 1 | 5.82E−06 | −0.6601629 | 7 | 0.7 | | |
| DMR11: 72648001 | 11 | 72648001 | 72650000 | 2000 | 1 | 3.42E−07 | −0.4059606 | 18 | 0.9 | PDE2A; AP003065.1 | Signaling |
| DMR11: 76945001 | 11 | 76945001 | 76946000 | 1000 | 1 | 1.93E−06 | −0.7038751 | 7 | 0.7 | ACER3; AP002498.1 | Metabolism |
| DMR11: 78428001 | 11 | 78428001 | 78431000 | 3000 | 1 | 1.93E−06 | −0.6269496 | 17 | 0.567 | GAB2; AP003086.2; AP003086.1; | Receptor; Transcription |
| | | | | | | | | | | NARS2 | |
| DMR11: 79350001 | 11 | 79350001 | 79352000 | 2000 | 1 | 9.99E−07 | −0.3244548 | 21 | 1.05 | TENM4 | Signaling |
| DMR11: 79876001 | 11 | 79876001 | 79877000 | 1000 | 1 | 3.53E−07 | 1.447963 | 6 | 0.6 | | |
| DMR11: 93175001 | 11 | 93175001 | 93176000 | 1000 | 1 | 9.74E−06 | −0.42818 | 12 | 1.2 | SLC36A4; AP003072.5 | Transport |
| DMR11: 94281001 | 11 | 94281001 | 94282000 | 1000 | 1 | 1.64E−07 | −0.8646871 | 9 | 0.9 | AP002784.1 | |
| DMR11: 95592001 | 11 | 95592001 | 95593000 | 1000 | 1 | 4.67E−07 | −0.730889 | 7 | 0.7 | AP000820.2 | |
| DMR11: 97733001 | 11 | 97733001 | 97737000 | 4000 | 1 | 3.85E−06 | −0.6907272 | 10 | 0.25 | | |
| DMR11: 106275001 | 11 | 106275001 | 106276000 | 1000 | 1 | 3.84E−06 | −0.7619225 | 6 | 0.6 | | |
| DMR11: 109564001 | 11 | 109564001 | 109565000 | 1000 | 1 | 6.72E−07 | −0.6557736 | 2 | 0.2 | AP003049.2 | |
| DMR11: 111872001 | 11 | 111872001 | 111873000 | 1000 | 1 | 4.27E−06 | −0.6308238 | 8 | 0.8 | ALG9; AP001781.2; GNG5P3; FDXACB1; | Metabolism |
| | | | | | | | | | | C11orf1 | |
| DMR11: 112401001 | 11 | 112401001 | 112403000 | 2000 | 1 | 2.73E−06 | −0.4219308 | 18 | 0.9 | AP003063.1 | |
| DMR11: 120177001 | 11 | 120177001 | 120179000 | 2000 | 1 | 3.39E−07 | 1.0832786 | 18 | 0.9 | TRIM29; AP000679.1 | Metabolism |
| DMR11: 121501001 | 11 | 121501001 | 121502000 | 1000 | 1 | 4.35E−06 | −0.7893128 | 6 | 0.6 | SORL1 | Receptor |
| DMR11: 128972001 | 11 | 128972001 | 128973000 | 1000 | 1 | 1.59E−06 | −0.5045185 | 10 | 1 | ARHGAP32 | Signaling |
| DMR12: 4746001 | 12 | 4746001 | 4748000 | 2000 | 1 | 3.52E−07 | 0.697345 | 14 | 0.7 | AC005833.1; GALNT8 | Metabolism |
| DMR12: 5142001 | 12 | 5142001 | 5143000 | 1000 | 1 | 3.22E−06 | −0.4060174 | 17 | 1.7 | | |
| DMR12: 7100001 | 12 | 7100001 | 7105000 | 5000 | 1 | 7.21E−06 | −0.4409029 | 58 | 1.16 | C1R; C1RL; C1RL-AS1 | Immune; Protease |
| DMR12: 9415001 | 12 | 9415001 | 9416000 | 1000 | 1 | 8.47E−06 | 0.4767515 | 21 | 2.1 | AC141557.1; AC141557.2; DDX12P | |
| DMR12: 9579001 | 12 | 9579001 | 9580000 | 1000 | 1 | 6.05E−06 | −1.3024967 | 8 | 0.8 | AC092821.3; AC092821.1 | |
| DMR12: 10651001 | 12 | 10651001 | 10653000 | 2000 | 1 | 1.93E−08 | −0.7747836 | 15 | 0.75 | STYK1 | Receptor |
| DMR12: 13564001 | 12 | 13564001 | 13565000 | 1000 | 1 | 5.08E−06 | −0.9862011 | 35 | 3.5 | GRIN2B | Receptor |
| DMR12: 19669001 | 12 | 19669001 | 19670000 | 1000 | 1 | 2.29E−07 | −0.8077415 | 3 | 0.3 | AEBP2 | Transcription |
| DMR12: 20061001 | 12 | 20061001 | 20062000 | 1000 | 1 | 5.69E−06 | −0.5184235 | 8 | 0.8 | LINC02398 | |
| DMR12: 27229001 | 12 | 27229001 | 27230000 | 1000 | 1 | 9.20E−06 | −0.4982085 | 3 | 0.3 | | |
| DMR12: 32916001 | 12 | 32916001 | 32917000 | 1000 | 1 | 3.67E−06 | −0.5129993 | 6 | 0.6 | | |
| DMR12: 43584001 | 12 | 43584001 | 43585000 | 1000 | 1 | 1.56E−06 | −0.5114725 | 10 | 1 | | |
| DMR12: 47009001 | 12 | 47009001 | 47010000 | 1000 | 1 | 9.47E−06 | −0.5548508 | 9 | 0.9 | | |
| DMR12: 49758001 | 12 | 49758001 | 49759000 | 1000 | 1 | 1.67E−06 | −0.6080902 | 10 | 1 | TMBIM6 | Apoptosis |
| DMR12: 53498001 | 12 | 53498001 | 53500000 | 2000 | 1 | 1.79E−06 | 0.8645961 | 61 | 3.05 | MAP3K12; AC023509.3; TARBP2; | Signaling; Transcription |
| | | | | | | | | | | ATF7; NPFF | |
| DMR12: 55811001 | 12 | 55811001 | 55812000 | 1000 | 1 | 6.24E−06 | −0.5246924 | 9 | 0.9 | SARNP; AC023055.1; ORMDL2; | Transcription; Translation; Protein |
| | | | | | | | | | | DNAJC14 | Binding |
| DMR12: 62445001 | 12 | 62445001 | 62446000 | 1000 | 1 | 2.93E−07 | −0.7140059 | 4 | 0.4 | | |
| DMR12: 68739001 | 12 | 68739001 | 68740000 | 1000 | 1 | 6.63E−06 | 0.8115642 | 20 | 2 | NUP107; SLC35 E3 | Transport |
| DMR12: 71103001 | 12 | 71103001 | 71104000 | 1000 | 1 | 7.41E−06 | 1.1122277 | 6 | 0.6 | AC025575.1; AC025575.2 | |
| DMR12: 75480001 | 12 | 75480001 | 75482000 | 2000 | 1 | 2.99E−06 | −0.5648618 | 21 | 1.05 | GLIPR1; AC121761.1; KRR1 | Transcription |
| DMR12: 81780001 | 12 | 81780001 | 81781000 | 1000 | 1 | 3.89E−06 | −1.1812637 | 4 | 0.4 | | |
| DMR12: 86917001 | 12 | 86917001 | 86919000 | 2000 | 1 | 5.12E−06 | −0.6624892 | 13 | 0.65 | | |
| DMR12: 89311001 | 12 | 89311001 | 89314000 | 3000 | 1 | 5.48E−06 | 0.6391671 | 34 | 1.133 | LINC02458; MRPS6P4 | |
| DMR12: 99154001 | 12 | 99154001 | 99155000 | 1000 | 1 | 4.73E−06 | −0.4861876 | 17 | 1.7 | ANKS1B | Receptor |
| DMR12: 101460001 | 12 | 101460001 | 101461000 | 1000 | 1 | 6.30E−06 | 0.5985655 | 16 | 1.6 | RNU5E-5P | |
| DMR12: 102110001 | 12 | 102110001 | 102112000 | 2000 | 1 | 8.81E−06 | −0.9481755 | 13 | 0.65 | NUP37; PARPBP | Metabolism |
| DMR12: 102176001 | 12 | 102176001 | 102177000 | 1000 | 1 | 4.75E−06 | 0.7412656 | 9 | 0.9 | PARPBP | |
| DMR12: 102498001 | 12 | 102498001 | 102499000 | 1000 | 1 | 4.99E−06 | −0.4957777 | 2 | 0.2 | | |
| DMR12: 105630001 | 12 | 105630001 | 105632000 | 2000 | 1 | 2.23E−06 | −0.4978292 | 15 | 0.75 | | |
| DMR12: 106215001 | 12 | 106215001 | 106216000 | 1000 | 1 | 2.79E−06 | 0.4783249 | 19 | 1.9 | | |
| DMR12: 109267001 | 12 | 109267001 | 109269000 | 2000 | 1 | 4.92E−06 | −0.3766094 | 33 | 1.65 | ACACB; FOXN4 | Metabolism; Transcription |
| DMR12: 110325001 | 12 | 110325001 | 110327000 | 2000 | 1 | 1.30E−07 | −0.9564508 | 18 | 0.9 | ATP2A2 | Metabolism |
| DMR12: 110336001 | 12 | 110336001 | 110337000 | 1000 | 1 | 5.90E−06 | −0.7809265 | 7 | 0.7 | ATP2A2 | Metabolism |
| DMR12: 110441001 | 12 | 110441001 | 110445000 | 4000 | 1 | 4.96E−07 | 0.7889969 | 75 | 1.875 | AC144548.1; ARPC3; GPN3 | Cytoskeleton; Transcription |
| DMR12: 112178001 | 12 | 112178001 | 112179000 | 1000 | 1 | 3.32E−06 | 0.868337 | 21 | 2.1 | HECTD4 | |
| DMR12: 114323001 | 12 | 114323001 | 114324000 | 1000 | 1 | 4.39E−06 | −0.6522382 | 11 | 1.1 | | |
| DMR12: 114875001 | 12 | 114875001 | 114879000 | 4000 | 1 | 3.57E−06 | 0.5413825 | 50 | 1.25 | | |
| DMR12: 119809001 | 12 | 119809001 | 119810000 | 1000 | 1 | 3.51E−06 | −0.5548171 | 11 | 1.1 | CIT | Signaling |
| DMR12: 121514001 | 12 | 121514001 | 121516000 | 2000 | 1 | 3.39E−07 | 0.8815815 | 34 | 1.7 | KDM2B | |
| DMR12: 123452001 | 12 | 123452001 | 123453000 | 1000 | 1 | 1.90E−06 | −0.4962994 | 9 | 0.9 | SNRNP35 | Translation |
| DMR12: 124005001 | 12 | 124005001 | 124006000 | 1000 | 1 | 8.23E−06 | 0.9747191 | 10 | 1 | ZNF664; RFLNA; AC068790.8; | Transcription |
| | | | | | | | | | | AC068790.1 | |
| DMR12: 125102001 | 12 | 125102001 | 125104000 | 2000 | 1 | 1.21E−06 | 0.725551 | 42 | 2.1 | AACS | Metabolism |
| DMR12: 125211001 | 12 | 125211001 | 125212000 | 1000 | 1 | 4.67E−06 | −0.6246062 | 9 | 0.9 | TMEM132B | Unknown |
| DMR12: 128045001 | 12 | 128045001 | 128046000 | 1000 | 1 | 5.88E−06 | 0.5920729 | 15 | 1.5 | | |
| DMR12: 128609001 | 12 | 128609001 | 128610000 | 1000 | 1 | 5.10E−06 | 0.7145798 | 6 | 0.6 | TMEM132C | Unknown |
| DMR12: 128663001 | 12 | 128663001 | 128666000 | 3000 | 1 | 1.78E−06 | −0.5331325 | 66 | 2.2 | TMEM132C | Unknown |
| DMR12: 128711001 | 12 | 128711001 | 128714000 | 3000 | 1 | 7.13E−06 | 0.7826136 | 51 | 1. | TMEM132C | Unknown |
| DMR12: 131432001 | 12 | 131432001 | 131436000 | 4000 | 1 | 8.59E−06 | 1.0151738 | 177 | 4.425 | AC073578.4 | |
| DMR12: 132316001 | 12 | 132316001 | 132317000 | 1000 | 1 | 4.95E−06 | −0.4315296 | 31 | 3.1 | GALNT9 | Metabolism |
| DMR12: 132731001 | 12 | 132731001 | 132734000 | 3000 | 1 | 1.20E−06 | 0.6993745 | 151 | 5.033 | PGAM5; RNA5SP379; ANKLE2 | |
| DMR13: 19292001 | 13 | 19292001 | 19293000 | 1000 | 1 | 2.74E−06 | −0.6175243 | 12 | 1.2 | ANKRD26P3 | |
| DMR13: 21960001 | 13 | 21960001 | 21962000 | 2000 | 1 | 9.31E−06 | −0.6041032 | 13 | 0.65 | | |
| DMR13: 26666001 | 13 | 26666001 | 26667000 | 1000 | 1 | 3.98E−06 | 0.5971087 | 16 | 1.6 | WASF3 | Cytoskeleton |
| DMR13: 26944001 | 13 | 26944001 | 26945000 | 1000 | 1 | 3.80E−06 | −0.5491039 | 5 | 0.5 | | |
| DMR13: 28996001 | 13 | 28996001 | 28999000 | 3000 | 1 | 3.18E−07 | −0.5977335 | 29 | 0.967 | MTUS2 | Cytoskeleton |
| DMR13: 36497001 | 13 | 36497001 | 36498000 | 1000 | 1 | 3.79E−06 | 1.036701 | 5 | 0.5 | HIST1H2APS6 | |
| DMR13: 44088001 | 13 | 44088001 | 44089000 | 1000 | 1 | 2.34E−06 | −0.6920414 | 8 | 0.8 | | |
| DMR13: 45282001 | 13 | 45282001 | 45284000 | 2000 | 1 | 2.40E−06 | 0.7220002 | 18 | 9 | GTF2F2 | Transcription |
| DMR13: 56816001 | 13 | 56816001 | 56818000 | 2000 | 1 | 1.38E−06 | −0.6806105 | 7 | 0.35 | | |
| DMR13: 59560001 | 13 | 59560001 | 59561000 | 1000 | 1 | 6.94E−06 | −0.5272228 | 3 | 0.3 | | |
| DMR13: 59957001 | 13 | 59957001 | 59958000 | 1000 | 1 | 7.18E−07 | −0.6158558 | 4 | 0.4 | DIAPH3 | Cytoskeleton |
| DMR13: 60867001 | 13 | 60867001 | 60869000 | 2000 | 1 | 1.10E−08 | −0.7810444 | 3 | 0.15 | | |
| DMR13: 65751001 | 13 | 65751001 | 65752000 | 1000 | 1 | 1.98E−06 | −0.9786599 | 2 | 0.2 | | |
| DMR13: 67510001 | 13 | 67510001 | 67511000 | 1000 | 1 | 9.08E−06 | −0.6254222 | 9 | 0.9 | | |
| DMR13: 69426001 | 13 | 69426001 | 69430000 | 4000 | 1 | 5.58E−06 | 0.5709394 | 20 | 0.5 | | |
| DMR13: 73229001 | 13 | 73229001 | 73231000 | 2000 | 1 | 3.07E−08 | 0.9013651 | 18 | 0.9 | RNY1P8 | |
| DMR13: 75109001 | 13 | 75109001 | 75112000 | 3000 | 1 | 7.76E−06 | −0.4875661 | 21 | 0.7 | RNU6-38P | |
| DMR13: 79970001 | 13 | 79970001 | 79972000 | 2000 | 1 | 1.69E−06 | −0.6902922 | 11 | 0.55 | AL158064.1 | |
| DMR13: 95689001 | 13 | 95689001 | 95690000 | 1000 | 1 | 2.64E−06 | −0.4309865 | 4 | 0.4 | DNAJC3; MTND5P2; MTND6P18; | Protein Binding |
| | | | | | | | | | | MTCYBP3 | |
| DMR13: 98266001 | 13 | 98266001 | 98267000 | 1000 | 1 | 6.40E−06 | 0.6814442 | 15 | 1.5 | FARP1 | Signaling |
| DMR13: 99311001 | 13 | 99311001 | 99316000 | 5000 | 1 | 5.06E−07 | −0.4679861 | 64 | 1.28 | UBAC2; GPR183 | Signaling |
| DMR13: 105201001 | 13 | 105201001 | 105202000 | 1000 | 1 | 4.30E−07 | −1.2078866 | 4 | 0.4 | | |
| DMR13: 107931001 | 13 | 107931001 | 107932000 | 1000 | 1 | 4.44E−07 | 0.7650271 | 5 | 0.5 | | |
| DMR13: 113470001 | 13 | 113470001 | 113471000 | 1000 | 1 | 2.93E−06 | −0.5195481 | 15 | 1.5 | DCUN1D2; DCUN1D2- | Proteolysis |
| | | | | | | | | | | AS; RNU1-16P | |
| DMR14: 24027001 | 14 | 24027001 | 24028000 | 1000 | 1 | 8.41E−06 | −0.5667869 | 3 | 0.3 | AL136419.1; DHRS4L1 | Metabolism |
| DMR14: 24415001 | 14 | 24415001 | 24416000 | 1000 | 1 | 1.05E−06 | −0.4544707 | 28 | 2.8 | NYNRIN | Transcription |
| DMR14: 26918001 | 14 | 26918001 | 26919000 | 1000 | 1 | 8.53E−06 | −1.0542583 | 9 | 0.9 | AL110292.1; MIR4307HG; MIR4307 | |
| DMR14: 27063001 | 14 | 27063001 | 27064000 | 1000 | 1 | 9.70E−06 | −0.675709 | 12 | 1.2 | AL110292.1 | |
| DMR14: 30687001 | 14 | 30687001 | 30689000 | 2000 | 1 | 4.95E−07 | −0.5831677 | 17 | 0.85 | SCFD1; UBE2CP1 | Receptor |
| DMR14: 34790001 | 14 | 34790001 | 34792000 | 2000 | 1 | 6.58E−06 | 0.6923428 | 19 | 0.95 | BAZ1A | Metabolism |
| DMR14: 48570001 | 14 | 48570001 | 48572000 | 2000 | 1 | 4.86E−06 | −0.7430836 | 17 | 0.85 | | |
| DMR14: 52035001 | 14 | 52035001 | 52037000 | 2000 | 1 | 2.24E−06 | −0.6898251 | 19 | 0.95 | NID2 | Extracellular Matrix |
| DMR14: 63549001 | 14 | 63549001 | 63551000 | 2000 | 1 | 4.26E−06 | 0.4715623 | 40 | 2 | PPP2R5E; AL136038.3 | Signaling |
| DMR14: 66714001 | 14 | 66714001 | 66716000 | 2000 | 1 | 2.42E−06 | −0.4104103 | 12 | 0.6 | GPHN | Receptor |
| DMR14: 67093001 | 14 | 67093001 | 67094000 | 1000 | 1 | 1.31E−06 | −0.9221725 | 3 | 0.3 | GPHN | Receptor |
| DMR14: 69838001 | 14 | 69838001 | 69840000 | 2000 | 1 | 2.99E−07 | 0.5105971 | 14 | 0.7 | | |
| DMR14: 76610001 | 14 | 76610001 | 76611000 | 1000 | 1 | 2.87E−07 | 1.1097524 | 6 | 0.6 | AC008050.1 | |
| DMR14: 79029001 | 14 | 79029001 | 79031000 | 2000 | 1 | 3.07E−07 | −0.7369459 | 10 | 0.5 | NRXN3 | Receptor |
| DMR14: 80091001 | 14 | 80091001 | 80092000 | 1000 | 1 | 4.87E−06 | −0.4813456 | 7 | 0.7 | | |
| DMR14: 86746001 | 14 | 86746001 | 86748000 | 2000 | 1 | 2.02E−06 | −0.6001955 | 11 | 0.55 | | |
| DMR14: 88290001 | 14 | 88290001 | 88292000 | 2000 | 1 | 2.92E−06 | −0.6365923 | 21 | 1.05 | KCNK10 | Transport |
| DMR14: 90106001 | 14 | 90106001 | 90110000 | 4000 | 1 | 7.56E−07 | 0.6835915 | 76 | 1.9 | KCNK13; GLRXP2 | Metabolism |
| DMR14: 90379001 | 14 | 90379001 | 90380000 | 1000 | 1 | 5.95E−06 | −0.4452028 | 8 | 0.8 | AL512791.2 | |
| DMR14: 92547001 | 14 | 92547001 | 92548000 | 1000 | 1 | 6.70E−06 | 0.9411342 | 6 | 0.6 | RIN3 | Signaling |
| DMR14: 95446001 | 14 | 95446001 | 95448000 | 2000 | 1 | 3.08E−07 | −0.5229343 | 30 | 1.5 | SYNE3 | |
| DMR14: 96645001 | 14 | 96645001 | 96648000 | 3000 | 1 | 7.46E−07 | −0.6205166 | 10 | 0.333 | RN7SKP108 | |
| DMR14: 99448001 | 14 | 99448001 | 99449000 | 1000 | 1 | 1.12E−06 | −0.4549786 | 13 | 1.3 | SETD3; RNU6-91P | |
| DMR14: 101827001 | 14 | 101827001 | 101828000 | 1000 | 1 | 9.70E−06 | 0.7856008 | 12 | 1.2 | PPP2R5C; AL137779.1 | Signaling |
| DMR14: 103701001 | 14 | 103701001 | 103702000 | 1000 | 1 | 1.39E−06 | 0.4950851 | 36 | 3.6 | KLC1; AL049840.2; AL049840.3; | Cytoskeleton; Transcription |
| | | | | | | | | | | AL049840.4; XRCC3 | |
| DMR14: 106189001 | 14 | 106189001 | 106190000 | 1000 | 1 | 3.29E−06 | 0.7841305 | 11 | 1.1 | SLC20A1P2; IGHV1-18; IGHV3-19 | |
| DMR15: 30831001 | 15 | 30831001 | 30833000 | 2000 | 1 | 3.79E−07 | −0.4597954 | 32 | 1.6 | HERC2P10 | |
| DMR15: 34840001 | 15 | 34840001 | 34843000 | 3000 | 1 | 8.34E−06 | −0.630889 | 23 | 0.767 | AC018868.2; AQR | Transcription |
| DMR15: 36684001 | 15 | 36684001 | 36688000 | 4000 | 1 | 6.49E−06 | −0.4764924 | 26 | 0.65 | C15orf41 | EST |
| DMR15: 40174001 | 15 | 40174001 | 40175000 | 1000 | 1 | 3.70E−07 | 0.7124244 | 14 | 1.4 | BUB1B | Signaling |
| DMR15: 40422001 | 15 | 40422001 | 40423000 | 1000 | 1 | 1.30E−06 | 0.6713205 | 48 | 4.8 | IVD | Metabolism |
| DMR15: 42047001 | 15 | 42047001 | 42048000 | 1000 | 1 | 6.07E−08 | −0.8507646 | 8 | 0.8 | PLA2G4E | Metabolism |
| DMR15: 42282001 | 15 | 42282001 | 42283000 | 1000 | 1 | 3.90E−06 | 1.0894247 | 10 | 1 | TMEM87A; GANC | Unknown; Metabolism |
| DMR15: 43596001 | 15 | 43596001 | 43599000 | 3000 | 1 | 1.34E−06 | −0.5614992 | 29 | 0.967 | PPIP5K1; CKMT1B; STRC; RNU6-554P | Signaling; Extracellular Matrix |
| DMR15: 44253001 | 15 | 44253001 | 44256000 | 3000 | 1 | 8.70E−06 | −0.4641334 | 29 | 0.967 | | |
| DMR15: 49400001 | 15 | 49400001 | 49401000 | 1000 | 1 | 5.26E−06 | −0.6751511 | 5 | 0.5 | FAM227B | |
| DMR15: 52365001 | 15 | 52365001 | 52366000 | 1000 | 1 | 1.70E−06 | −0.6316008 | 7 | 0.7 | MYO5A | Cytoskeleton |
| DMR15: 53743001 | 15 | 53743001 | 53744000 | 1000 | 1 | 5.22E−06 | 0.9684616 | 20 | 2 | WDR72 | |
| DMR15: 61121001 | 15 | 61121001 | 61124000 | 3000 | 1 | 3.76E−06 | −1.1658575 | 35 | 1.167 | RORA | Receptor |
| DMR15: 62743001 | 15 | 62743001 | 62744000 | 1000 | 1 | 6.34E−06 | −0.8823453 | 9 | 0.9 | TLN2 | Cytoskeleton |
| DMR15: 62963001 | 15 | 62963001 | 62966000 | 3000 | 1 | 9.65E−08 | −0.6067509 | 39 | 1.3 | AC079328.1 | |
| DMR15: 64230001 | 15 | 64230001 | 64231000 | 1000 | 1 | 4.83E−06 | 0.4355004 | 21 | 2.1 | CSNK1G1; AC087632.1 | Signaling |
| DMR15: 66241001 | 15 | 66241001 | 66242000 | 1000 | 1 | 1.99E−09 | −0.6412479 | 13 | 1.3 | MEGF11 | Extracellular Matrix |
| DMR15: 68771001 | 15 | 68771001 | 68772000 | 1000 | 1 | 2.89E−06 | −0.4701824 | 5 | 0.5 | ANP32A | Signaling |
| DMR15: 70433001 | 15 | 70433001 | 70434000 | 1000 | 1 | 1.79E−07 | −0.5189136 | 18 | 1.8 | | |
| DMR15: 70529001 | 15 | 70529001 | 70532000 | 3000 | 1 | 6.97E−06 | −0.3902115 | 25 | 0.833 | | |
| DMR15: 71213001 | 15 | 71213001 | 71214000 | 1000 | 1 | 8.58E−07 | −1.1917199 | 6 | 0.6 | THSD4 | Extracellular Matrix |
| DMR15: 75185001 | 15 | 75185001 | 75186000 | 1000 | 1 | 7.13E−06 | 0.5836484 | 17 | 1.7 | RPL36AP45; C15orf39 | Growth Factors & Cytokines |
| DMR15: 77892001 | 15 | 77892001 | 77893000 | 1000 | 1 | 4.85E−06 | 0.5930174 | 16 | 1.6 | CSPG4P13 | |
| DMR15: 77963001 | 15 | 77963001 | 77964000 | 1000 | 1 | 3.35E−06 | −0.6939627 | 8 | 0.8 | AC104758.5 | |
| DMR15: 78209001 | 15 | 78209001 | 78211000 | 2000 | 1 | 6.59E−06 | 0.5237414 | 36 | 1.8 | ACSBG1 | Metabolism |
| DMR15: 78624001 | 15 | 78624001 | 78626000 | 2000 | 1 | 7.74E−06 | −0.360405 | 29 | 1.45 | CHRNA3; CHRNB4; AC067863.1 | Receptor |
| DMR15: 78920001 | 15 | 78920001 | 78921000 | 1000 | 1 | 3.45E−06 | 0.984213 | 8 | 0.8 | CTSH | Protease |
| DMR15: 86743001 | 15 | 86743001 | 86744000 | 1000 | 1 | 7.44E−06 | −0.6118818 | 10 | 1 | AGBL1 | Signaling |
| DMR15: 87023001 | 15 | 87023001 | 87026000 | 3000 | 1 | 8.07E−09 | −0.7509167 | 9 | 0.3 | AGBL1 | Signaling |
| DMR15: 91262001 | 15 | 91262001 | 91264000 | 2000 | 1 | 2.19E−06 | −0.3980937 | 28 | 1.4 | SV2B | Binding Protein |
| DMR15: 98978001 | 15 | 98978001 | 98980000 | 2000 | 1 | 1.95E−06 | 0.7588471 | 34 | 1.7 | AC036108.1; PGPEP1L | Protease |
| DMR15: 101548001 | 15 | 101548001 | 101550000 | 2000 | 1 | 2.53E−06 | −0.4412748 | 38 | 1.9 | | |
| DMR16: 3791001 | 16 | 3791001 | 3792000 | 1000 | 1 | 8.93E−07 | −0.4352327 | 24 | 2.4 | CREBBP | Transcription |
| DMR16: 5472001 | 16 | 5472001 | 5475000 | 3000 | 1 | 2.42E−06 | 0.5231262 | 35 | 1.167 | RBFOX1 | Unknown |
| DMR16: 6397001 | 16 | 6397001 | 6399000 | 2000 | 1 | 4.39E−06 | −0.8114704 | 16 | 0.8 | RBFOX1 | Unknown |
| DMR16: 6851001 | 16 | 6851001 | 6852000 | 1000 | 1 | 4.59E−06 | −0.563282 | 11 | 1.1 | RBFOX1 | Unknown |
| DMR16: 6971001 | 16 | 6971001 | 6972000 | 1000 | 1 | 2.10E−06 | −0.3482249 | 14 | 1.4 | RBFOX1 | Unknown |
| DMR16: 11992001 | 16 | 11992001 | 11995000 | 3000 | 1 | 1.99E−06 | 0.5223881 | 37 | 1.233 | SNX29 | Cytoskeleton |
| DMR16: 12633001 | 16 | 12633001 | 12634000 | 1000 | 1 | 4.43E−06 | 0.6979455 | 18 | 1.8 | | |
| DMR16: 19052001 | 16 | 19052001 | 19053000 | 1000 | 1 | 1.12E−06 | 0.6511852 | 15 | 1.5 | TMC7; AC099518.4 | Transport |
| DMR16: 24437001 | 16 | 24437001 | 24440000 | 3000 | 1 | 5.38E−06 | −0.7692339 | 19 | 0.633 | | |
| DMR16: 27243001 | 16 | 27243001 | 27244000 | 1000 | 1 | 4.77E−06 | −0.4181808 | 16 | 1.6 | NSMCE1 | |
| DMR16: 28616001 | 16 | 28616001 | 28617000 | 1000 | 1 | 1.97E−06 | −0.4645907 | 22 | 2.2 | SULT1A1; AC020765.5 | Metabolism |
| DMR16: 31116001 | 16 | 31116001 | 31117000 | 1000 | 1 | 1.98E−06 | 0.6873786 | 60 | 6 | BCKDK; KAT8; AC135050.5; AC135050.6 | Signaling; Epigenetic |
| DMR16: 46538001 | 16 | 46538001 | 46540000 | 2000 | 1 | 5.53E−06 | −0.4745983 | 13 | 0.65 | ANKRD26P1 | |
| DMR16: 55737001 | 16 | 55737001 | 55738000 | 1000 | 1 | 3.91E−06 | −0.4457135 | 7 | 0.7 | CES1P2 | |
| DMR16: 56968001 | 16 | 56968001 | 56969000 | 1000 | 1 | 3.35E−06 | −0.4016815 | 14 | 1.4 | CETP | Binding Protein |
| DMR16: 66282001 | 16 | 66282001 | 66283000 | 1000 | 1 | 8.56E−07 | −0.9281135 | 4 | 0.4 | | |
| DMR16: 68303001 | 16 | 68303001 | 68305000 | 2000 | 1 | 1.43E−06 | 0.642697 | 29 | 1.45 | SLC7A6; SLC7A6OS; PRMT7 | Metabolism |
| DMR16: 75102001 | 16 | 75102001 | 75103000 | 1000 | 1 | 8.16E−06 | −0.3433842 | 21 | 2.1 | ZNRF1; AC099508.1; LDHD | Transcription; Metabolism |
| DMR16: 75181001 | 16 | 75181001 | 75182000 | 1000 | 1 | 4.54E−06 | −0.7466281 | 6 | 0.6 | ZFP1 | |
| DMR16: 76252001 | 16 | 76252001 | 76255000 | 3000 | 1 | 4.04E−06 | −0.4643694 | 35 | 1.167 | AC010528.1 | |
| DMR16: 77339001 | 16 | 77339001 | 77340000 | 1000 | 1 | 8.60E−07 | −0.785832 | 8 | 0.8 | ADAMTS18 | Protease |
| DMR16: 87824001 | 16 | 87824001 | 87825000 | 1000 | 1 | 3.42E−06 | −0.5329661 | 20 | 2 | SLC7A5; MIR6775 | Metabolism |
| DMR16: 87970001 | 16 | 87970001 | 87973000 | 3000 | 1 | 5.79E−06 | −0.3894862 | 42 | 1.4 | BANP | Transcription |
| DMR16: 89402001 | 16 | 89402001 | 89403000 | 1000 | 1 | 5.94E−06 | 0.4520241 | 28 | 2.8 | ANKRD11 | EST |
| DMR16: 89713001 | 16 | 89713001 | 89714000 | 1000 | 1 | 4.47E−06 | 0.5205058 | 32 | 3.2 | VPS9D1; VPS9D1-AS1; ZNF276 | Transcription |
| DMR17: 963001 | 17 | 963001 | 964000 | 1000 | 1 | 8.05E−10 | 0.5073656 | 27 | 2.7 | NXN; AC036164.1 | Signaling |
| DMR17: 6433001 | 17 | 6433001 | 6434000 | 1000 | 1 | 2.06E−06 | −0.3925399 | 25 | 2.5 | AIPL1 | Protein Binding |
| DMR17: 6984001 | 17 | 6984001 | 6985000 | 1000 | 1 | 1.41E−06 | 0.6759975 | 13 | 1.3 | ALOX12-AS1; AC027763.2; AC040977.2; | |
| | | | | | | | | | | AC040977.1 | |
| DMR17: 7838001 | 17 | 7838001 | 7839000 | 1000 | 1 | 2.42E−07 | 1.1202783 | 12 | 1.2 | DNAH2; KDM6B | Cytoskeleton; Transcription |
| DMR17: 8909001 | 17 | 8909001 | 8911000 | 2000 | 1 | 1.70E−06 | −0.5784843 | 34 | 1.7 | PIK3R5 | Signaling |
| DMR17: 9707001 | 17 | 9707001 | 9708000 | 1000 | 1 | 9.47E−06 | 0.6569972 | 22 | 2.2 | USP43 | |
| DMR17: 10595001 | 17 | 10595001 | 10597000 | 2000 | 1 | 5.43E−06 | 0.8541415 | 19 | 0.95 | MYHAS; AC005323.1 | |
| DMR17: 11763001 | 17 | 11763001 | 11765000 | 2000 | 1 | 1.26E−08 | −0.736115 | 21 | 1.05 | DNAH9 | Cytoskeleton |
| DMR17: 11863001 | 17 | 11863001 | 11865000 | 2000 | 1 | 3.77E−07 | −0.5703788 | 13 | 0.65 | DNAH9; AC005209.1 | Cytoskeleton |
| DMR17: 16245001 | 17 | 16245001 | 16246000 | 1000 | 1 | 7.66E−06 | 0.4462797 | 23 | 2.3 | PIGL | Metabolism |
| DMR17: 18278001 | 17 | 18278001 | 18280000 | 2000 | 1 | 1.94E−06 | 0.5858592 | 38 | 1.9 | AC127537.1; TOP3A | Transcription |
| DMR17: 28031001 | 17 | 28031001 | 28033000 | 2000 | 1 | 4.47E−06 | −0.6934768 | 18 | 0.9 | AC090287.1; RF00156; NLK | Signaling |
| DMR17: 30727001 | 17 | 30727001 | 30728000 | 1000 | 1 | 7.92E−07 | −0.4150466 | 20 | 2 | SUZ12P1; AC127024.7; AC127024.3; AC12 7024.2 | |
| DMR17: 33222001 | 17 | 33222001 | 33223000 | 1000 | 1 | 1.04E−06 | −0.8983157 | 1 | 0.1 | ASIC2 | Transport |
| DMR17: 34755001 | 17 | 34755001 | 34756000 | 1000 | 1 | 2.17E−06 | −0.4877424 | 12 | 1.2 | | |
| DMR17: 35682001 | 17 | 35682001 | 35684000 | 2000 | 1 | 6.58E−06 | 0.9483375 | 25 | 1.25 | AP2B1 | Transport |
| DMR17: 36222001 | 17 | 36222001 | 36224000 | 2000 | 1 | 7.79E−09 | 0.9564993 | 26 | 1.3 | CCL4L2; AC243829.5 | |
| DMR17: 36740001 | 17 | 36740001 | 36741000 | 1000 | 1 | 4.32E−07 | −0.4195057 | 18 | 1.8 | | |
| DMR17: 40506001 | 17 | 40506001 | 40508000 | 2000 | 1 | 8.91E−06 | 0.634816 | 18 | 0.9 | TNS4; AC004585.1 | Signaling |
| DMR17: 40846001 | 17 | 40846001 | 40847000 | 1000 | 1 | 4.02E−06 | −0.4223231 | 8 | 0.8 | TMEM99; AC004231.3 | |
| DMR17: 42436001 | 17 | 42436001 | 42439000 | 3000 | 1 | 3.26E−06 | 0.5821806 | 46 | 1.533 | RNU7-97P | |
| DMR17: 43109001 | 17 | 43109001 | 43112000 | 3000 | 1 | 3.20E−06 | 0.5820137 | 54 | 1.8 | BRCA1 | Transcription |
| DMR17: 44496001 | 17 | 44496001 | 44497000 | 1000 | 1 | 2.48E−06 | 0.8977599 | 12 | 1.2 | GPATCH8; AC103703.1 | |
| DMR17: 50774001 | 17 | 50774001 | 50776000 | 2000 | 1 | 3.92E−07 | 0.6612705 | 43 | 2.15 | ANKRD40CL; MIR8059; AC005921.4 | |
| DMR17: 55556001 | 17 | 55556001 | 55558000 | 2000 | 1 | 7.06E−07 | −0.5026078 | 24 | 1.2 | AC105021.1; GARSP1; RNU6-1249P | |
| DMR17: 60736001 | 17 | 60736001 | 60738000 | 2000 | 1 | 2.22E−07 | 0.5793683 | 19 | 0.95 | BCAS3; AC110602.1 | Transcription |
| DMR17: 61238001 | 17 | 61238001 | 61239000 | 1000 | 1 | 1.97E−06 | 0.8992406 | 19 | 1.9 | BCAS3 | Transcription |
| DMR17: 63464001 | 17 | 63464001 | 63465000 | 1000 | 1 | 2.09E−06 | −0.6950074 | 5 | 0.5 | AC005828.5; PPIAP55 | |
| DMR17: 64998001 | 17 | 64998001 | 64999000 | 1000 | 1 | 2.28E−06 | 0.494202 | 18 | 1.8 | | |
| DMR17: 67168001 | 17 | 67168001 | 67169000 | 1000 | 1 | 8.02E−07 | 0.7367953 | 11 | 1.1 | HELZ | Transcription |
| DMR17: 67631001 | 17 | 67631001 | 67634000 | 3000 | 1 | 7.11E−07 | −0.5059349 | 61 | 2.033 | PITPNC1; AC079331.1 | |
| DMR17: 68913001 | 17 | 68913001 | 68914000 | 1000 | 1 | 1.15E−08 | −0.6930886 | 6 | 0.6 | ABCA8 | Receptor |
| DMR17: 73378001 | 17 | 73378001 | 73379000 | 1000 | 1 | 1.80E−06 | 0.5528568 | 16 | 1.6 | SDK2 | Development |
| DMR17: 73741001 | 17 | 73741001 | 73742000 | 1000 | 1 | 2.61E−06 | −0.4850082 | 19 | 1.9 | AC125421.1; LINC00469 | |
| DMR17: 75194001 | 17 | 75194001 | 75195000 | 1000 | 1 | 4.19E−06 | 0.5828536 | 45 | 4.5 | | |
| DMR17: 75537001 | 17 | 75537001 | 75538000 | 1000 | 1 | 3.32E−07 | 0.621861 | 18 | 1.8 | LLGL2 | Development |
| DMR17: 76900001 | 17 | 76900001 | 76902000 | 2000 | 1 | 4.88E−06 | −0.3985727 | 36 | 1.8 | MGAT5B | Metabolism |
| DMR17: 77598001 | 17 | 77598001 | 77599000 | 1000 | 1 | 8.09E−06 | 0.7931789 | 16 | 1.6 | AC021683.5 | |
| DMR17: 78336001 | 17 | 78336001 | 78337000 | 1000 | 1 | 4.25E−06 | 0.5847373 | 15 | 1.5 | AC087645.2 | |
| DMR18: 3760001 | 18 | 3760001 | 3762000 | 2000 | 1 | 9.46E−06 | −0.4058592 | 28 | 1.4 | DLGAP1; AP002478.2 | Signaling |
| DMR18: 8749001 | 18 | 8749001 | 8750000 | 1000 | 1 | 3.17E−06 | −0.5126872 | 11 | 1.1 | MTCL1 | |
| DMR18: 9902001 | 18 | 9902001 | 9903000 | 1000 | 1 | 5.82E−06 | 0.4980064 | 23 | 2.3 | AC006238.1 | |
| DMR18: 10568001 | 18 | 10568001 | 10569000 | 1000 | 1 | 5.04E−07 | 1.172583 | 10 | 1 | | |
| DMR18: 21429001 | 18 | 21429001 | 21430000 | 1000 | 1 | 4.35E−06 | 0.6792457 | 5 | 0.5 | GREB1L; AC015878.1 | |
| DMR18: 22336001 | 18 | 22336001 | 22337000 | 1000 | 1 | 1.66E−08 | −0.5445338 | 17 | 1.7 | | |
| DMR18: 26821001 | 18 | 26821001 | 26822000 | 1000 | 1 | 1.04E−06 | 1.0010425 | 2 | 0.2 | AQP4-AS1; AC018371.1; AC018371.2 | |
| DMR18: 31938001 | 18 | 31938001 | 31939000 | 1000 | 1 | 1.18E−06 | 1.0044881 | 5 | 0.5 | TRAPPC8; RNU6-1050P; AC009831.1 | |
| DMR18: 35725001 | 18 | 35725001 | 35727000 | 2000 | 1 | 1.72E−06 | −0.5902583 | 8 | 0.4 | AC090229.1 | |
| DMR18: 55250001 | 18 | 55250001 | 55252000 | 2000 | 1 | 7.32E−06 | −0.6984778 | 24 | 1.2 | TCF4 | Transcription |
| DMR18: 56496001 | 18 | 56496001 | 56498000 | 2000 | 1 | 3.03E−06 | −0.5625773 | 14 | 0.7 | | |
| DMR18: 56968001 | 18 | 56968001 | 56969000 | 1000 | 1 | 5.66E−06 | −0.4509659 | 5 | 0.5 | WDR7 | Unknown |
| DMR18: 58343001 | 18 | 58343001 | 58344000 | 1000 | 1 | 4.45E−06 | 0.6257183 | 15 | 1.5 | NEDD4L | Protease |
| DMR18: 62436001 | 18 | 62436001 | 62438000 | 2000 | 1 | 6.86E−06 | −0.7114954 | 34 | 1.7 | ACTBP9 | |
| DMR18: 63599001 | 18 | 63599001 | 63601000 | 2000 | 1 | 9.61E−06 | −0.8856543 | 15 | 0.75 | SERPINB13 | Protease |
| DMR18: 67261001 | 18 | 67261001 | 67263000 | 2000 | 1 | 7.94E−07 | −0.7200381 | 13 | 0.65 | | |
| DMR18: 67302001 | 18 | 67302001 | 67303000 | 1000 | 1 | 6.31E−07 | −1.1893104 | 6 | 0.6 | | |
| DMR18: 73132001 | 18 | 73132001 | 73133000 | 1000 | 1 | 1.40E−06 | −0.5854772 | 3 | 0.3 | | |
| DMR18: 74204001 | 18 | 74204001 | 74205000 | 1000 | 1 | 4.98E−06 | 0.8467968 | 10 | 1 | AC090398.1 | |
| DMR18: 78053001 | 18 | 78053001 | 78054000 | 1000 | 1 | 5.01E−06 | −0.4758 | 1 | 1.7 | | |
| DMR19: 603001 | 19 | 603001 | 605000 | 2000 | 1 | 6.20E−06 | 0.7706511 | 75 | 3.75 | HCN2 | Metabolism |
| DMR19: 862001 | 19 | 862001 | 864000 | 2000 | 1 | 7.70E−06 | 0.8872191 | 103 | 5.15 | ELANE; CFD; MED16 | Protease; Immune; Transcription |
| DMR19: 3230001 | 19 | 3230001 | 3231000 | 1000 | 1 | 6.08E−08 | −0.5573396 | 10 | 1 | CELF5 | Translation |
| DMR19: 7500001 | 19 | 7500001 | 7504000 | 4000 | 1 | 7.11E−06 | 0.6057991 | 151 | 3.775 | PEX11G; TEX45; AC008878.1 | |
| DMR19: 7599001 | 19 | 7599001 | 7601000 | 2000 | 1 | 4.15E−06 | 0.6055752 | 22 | 1.1 | CAMSAP3 | |
| DMR19: 7830001 | 19 | 7830001 | 7831000 | 1000 | 1 | 9.26E−06 | 0.6855874 | 85 | 8.5 | EVI5L | |
| DMR19: 10197001 | 19 | 10197001 | 10199000 | 2000 | 1 | 3.21E−06 | 0.7194797 | 31 | 1.55 | DNMT1 | Epigenetic |
| DMR19: 13626001 | 19 | 13626001 | 13627000 | 1000 | 1 | 6.20E−08 | 0.5860472 | 15 | 1.5 | CACNA1A | Transport |
| DMR19: 13800001 | 19 | 13800001 | 13801000 | 1000 | 1 | 7.08E−06 | 0.4843254 | 41 | 4.1 | ZSWIM4; AC020916.2 | Transcription |
| DMR19: 16948001 | 19 | 16948001 | 16950000 | 2000 | 1 | 2.06E−07 | 0.7652186 | 21 | 1.05 | CPAMD8 | |
| DMR19: 18085001 | 19 | 18085001 | 18087000 | 2000 | 1 | 1.27E−06 | −0.5950983 | 29 | 1.45 | IL12RB1 | |
| DMR19: 21043001 | 19 | 21043001 | 21044000 | 1000 | 1 | 4.89E−06 | −0.427829 | 7 | 0.7 | ZNF430 | Transcription |
| DMR19: 27955001 | 19 | 27955001 | 27956000 | 1000 | 1 | 8.25E−06 | −0.5328916 | 6 | 0.6 | AC006504.7; AC005357.2 | |
| DMR19: 29076001 | 19 | 29076001 | 29078000 | 2000 | 1 | 5.28E−06 | −0.2891072 | 27 | 1.35 | | |
| DMR19: 31144001 | 19 | 31144001 | 31145000 | 1000 | 1 | 1.69E−06 | −0.4941799 | 8 | 0.8 | AC020912.1; TSHZ3 | Transcription |
| DMR19: 33107001 | 19 | 33107001 | 33110000 | 3000 | 1 | 7.18E−06 | −0.4886915 | 60 | 2 | GPATCH1 | |
| DMR19: 36978001 | 19 | 36978001 | 36980000 | 2000 | 1 | 8.79E−06 | −0.5872276 | 23 | 1.15 | ZNF568 | Transcription |
| DMR19: 39861001 | 19 | 39861001 | 39862000 | 1000 | 1 | 1.19E−07 | 0.7759882 | 8 | 0.8 | FCGBP | Extracellular Matrix |
| DMR19: 42889001 | 19 | 42889001 | 42891000 | 2000 | 1 | 1.02E−06 | −0.5300016 | 34 | 1.7 | PSG1 | Extracellular Matrix |
| DMR19: 44922001 | 19 | 44922001 | 44924000 | 2000 | 1 | 9.88E−06 | 0.4064002 | 42 | 2.1 | AC011481.4; APOC1; APOC1P1 | Transport |
| DMR19: 46318001 | 19 | 46318001 | 46319000 | 1000 | 1 | 1.24E−07 | 0.8315254 | 15 | 1.5 | HIF3A; AC007193.2 | Transcription |
| DMR19: 50906001 | 19 | 50906001 | 50907000 | 1000 | 1 | 6.16E−06 | 0.6470385 | 8 | 0.8 | KLKP1; KLK4 | Protease |
| DMR20: 9582001 | 20 | 9582001 | 9583000 | 1000 | 1 | 6.46E−06 | −0.7489359 | 6 | 0.6 | PAK5; AL353612.1 | |
| DMR20: 11768001 | 20 | 11768001 | 11770000 | 2000 | 1 | 1.93E−06 | −0.6202546 | 14 | 0.7 | | |
| DMR20: 13703001 | 20 | 13703001 | 13704000 | 1000 | 1 | 4.88E−08 | 0.8215851 | 13 | 1.3 | | |
| DMR20: 15075001 | 20 | 15075001 | 15076000 | 1000 | 1 | 4.59E−06 | −0.6158566 | 7 | 0.7 | MACROD2 | |
| DMR20: 18989001 | 20 | 18989001 | 18990000 | 1000 | 1 | 5.57E−06 | −0.8119199 | 7 | 0.7 | | |
| DMR20: 22597001 | 20 | 22597001 | 22600000 | 3000 | 1 | 1.82E−06 | −0.5801319 | 29 | 0.967 | LNCNEF | |
| DMR20: 23455001 | 20 | 23455001 | 23456000 | 1000 | 1 | 3.68E−07 | −0.587544 | 11 | 1.1 | CST11; RF00019; AL109954.2 | Signaling |
| DMR20: 26117001 | 20 | 26117001 | 26118000 | 1000 | 1 | 7.19E−06 | −0.4663103 | 12 | 1.2 | NCOR1P1 | |
| DMR20: 34042001 | 20 | 34042001 | 34043000 | 1000 | 1 | 9.08E−06 | 0.4914578 | 16 | 1.6 | RALY; MIR4755 | Transcription |
| DMR20: 36082001 | 20 | 36082001 | 36084000 | 2000 | 1 | 5.12E−06 | 0.8193762 | 22 | 1.1 | AL035420.1; HMGB3P2; AL035420.2; | |
| | | | | | | | | | | EPB 41L1 | |
| DMR20: 39079001 | 20 | 39079001 | 39080000 | 1000 | 1 | 3.50E−06 | 0.9926674 | 17 | 1.7 | | |
| DMR20: 47404001 | 20 | 47404001 | 47405000 | 1000 | 1 | 1.44E−06 | 0.5621608 | 14 | 1.4 | LINC01754 | |
| DMR20: 47941001 | 20 | 47941001 | 47942000 | 1000 | 1 | 2.38E−07 | −0.484401 | 21 | 2.1 | AL357558.2 | |
| DMR20: 48875001 | 20 | 48875001 | 48877000 | 2000 | 1 | 3.41E−06 | −0.6567095 | 32 | 1.6 | | |
| DMR20: 52445001 | 20 | 52445001 | 52447000 | 2000 | 1 | 1.88E−06 | −0.3016192 | 23 | 1.15 | LINC01524; AL109610.1 | |
| 20: 53996001 | 20 | 53996001 | 53997000 | 1000 | 1 | 2.25E−07 | −0.4277728 | 11 | 1.1 | BCAS1 | |
| 20: 55763001 | 20 | 55763001 | 55764000 | 1000 | 1 | 5.23E−06 | −0.5593399 | 7 | 0.7 | | |
| 20: 55944001 | 20 | 55944001 | 55945000 | 1000 | 1 | 2.32E−06 | 1.1446512 | 8 | 0.8 | | |
| 20: 61483001 | 20 | 61483001 | 61484000 | 1000 | 1 | 4.29E−08 | −0.5607195 | 16 | 1.6 | CDH4 | Extracellular Matrix |
| 20: 64093001 | 20 | 64093001 | 64095000 | 2000 | 1 | 4.08E−06 | 0.8474669 | 26 | 1.3 | OPRL1; LKAAEAR1; MYT1 | Receptor; Transcription |
| 21: 6152001 | 21 | 6152001 | 6153000 | 1000 | 1 | 8.97E−06 | −0.4006685 | 2 | 2.1 | | |
| 21: 6342001 | 21 | 6342001 | 6344000 | 2000 | 1 | 9.96E−06 | −0.5381794 | 17 | 0.85 | CU633906.2 | |
| 21: 10741001 | 21 | 10741001 | 10743000 | 2000 | 1 | 6.11E−06 | −0.5337132 | 16 | 0.8 | | |
| 21: 17965001 | 21 | 17965001 | 17966000 | 1000 | 1 | 2.16E−06 | 0.6491482 | 4 | 0.4 | CHODL | |
| 21: 19680001 | 21 | 19680001 | 19681000 | 1000 | 1 | 5.08E−07 | −0.7726042 | 5 | 0.5 | | |
| 21: 23640001 | 21 | 23640001 | 23641000 | 1000 | 1 | 2.15E−06 | −0.9618059 | 6 | 0.6 | | |
| 21: 23835001 | 21 | 23835001 | 23836000 | 1000 | 1 | 3.71E−08 | 1.1225167 | 11 | 1.1 | | |
| 21: 26824001 | 21 | 26824001 | 26825000 | 1000 | 1 | 2.31E−06 | 0.8413207 | 26 | 2.6 | | |
| 21: 31508001 | 21 | 31508001 | 31509000 | 1000 | 1 | 8.63E−06 | −0.4623201 | 19 | 1.9 | TIAM1 | Transcription |
| 21: 33493001 | 21 | 33493001 | 33494000 | 1000 | 1 | 5.79E−06 | 0.5975765 | 29 | 2.9 | AP000302.1; DNAJC28; GART | Transcription; Metabolism |
| 21: 38356001 | 21 | 38356001 | 38357000 | 1000 | 1 | 9.45E−08 | 0.4944057 | 22 | 2.2 | | |
| 21: 43583001 | 21 | 43583001 | 43585000 | 2000 | 1 | 3.04E−07 | −0.5202146 | 12 | 0.6 | HSF2BP | |
| 21: 46148001 | 21 | 46148001 | 46149000 | 1000 | 1 | 5.34E−07 | −0.5375053 | 17 | 1.7 | FTCD; FTCD-AS1 | |
| 22: 17210001 | 22 | 17210001 | 17212000 | 2000 | 1 | 8.66E−06 | 0.4740007 | 51 | 2.55 | ADA2; FAM32BP | Transcription |
| 22: 18386001 | 22 | 18386001 | 18388000 | 2000 | 1 | 5.01E−06 | 0.7548866 | 11 | 0.55 | FAM230J | |
| 22: 29607001 | 22 | 29607001 | 29609000 | 2000 | 1 | 9.53E−06 | 0.4201027 | 30 | 1.5 | NF2; RPEP4 | Cytoskeleton |
| 22: 34327001 | 22 | 34327001 | 34328000 | 1000 | 1 | 1.63E−06 | −1.0055776 | 4 | 0.4 | | |
| 22: 39704001 | 22 | 39704001 | 39706000 | 2000 | 1 | 3.50E−07 | 0.5982911 | 49 | 2.45 | | |
| 22: 46325001 | 22 | 46325001 | 46329000 | 4000 | 1 | 6.89E−06 | 0.428583 | 91 | 2.275 | GTSE1; TRMU | |
| 22: 49957001 | 22 | 49957001 | 49958000 | 1000 | 1 | 5.09E−07 | −0.4575814 | 23 | 2.3 | PIM3; MIR6821 | Epigenetic |
| X: 1364001 | x | 1364001 | 1369000 | 5000 | 1 | 4.56E−06 | 0.9289401 | 494 | 9.88 | IL3RA | Receptor |
| X: 13651001 | X | 13651001 | 13653000 | 2000 | 1 | 6.31E−06 | 1.0192184 | 30 | 1.5 | TCEANC | Transcription |
| X: 16276001 | X | 16276001 | 16277000 | 1000 | 1 | 5.26E−06 | −0.6609142 | 4 | 0.4 | | |
| X: 17491001 | X | 17491001 | 17492000 | 1000 | 1 | 3.02E−07 | −0.6759206 | 10 | 1 | NHS | |
| X: 22781001 | X | 22781001 | 22782000 | 1000 | 1 | 7.68E−06 | −0.8046573 | 5 | 0.5 | PTCHD1-AS | |
| X: 23695001 | X | 23695001 | 23697000 | 2000 | 1 | 2.79E−06 | 0.8706407 | 42 | 2.1 | PRDX4; ACOT9 | Electron Transport; Metabolism |
| X: 24165001 | X | 24165001 | 24166000 | 1000 | 1 | 2.98E−06 | 0.9300575 | 13 | 1.3 | ZFX | Transcription |
| X: 31900001 | x | 31900001 | 31901000 | 1000 | 1 | 3.63E−06 | −0.9599591 | 13 | 1.3 | DMD | Development |
| X: 44792001 | X | 44792001 | 44793000 | 1000 | 1 | 5.94E−06 | 0.6076124 | 11 | 1.1 | | |
| X: 46370001 | X | 46370001 | 46372000 | 2000 | 1 | 3.53E−06 | 1.0238952 | 34 | 1.7 | | |
| X: 46581001 | X | 46581001 | 46582000 | 1000 | 1 | 2.05E−08 | 0.6105555 | 27 | 2.7 | CHST7 | Metabolism |
| X: 51310001 | x | 51310001 | 51312000 | 2000 | 1 | 9.77E−06 | −0.9422977 | 18 | 0.9 | | |
| X: 52488001 | X | 52488001 | 52489000 | 1000 | 1 | 2.93E−06 | −0.5199447 | 18 | 1.8 | BX510359.8; BX510359.7; RBM22P6; | |
| | | | | | | | | | | XAGE1A | |
| X: 53469001 | X | 53469001 | 53470000 | 1000 | 1 | 8.43E−06 | 0.9332251 | 7 | 0.7 | VTRNA3-1P | |
| X: 72115001 | X | 72115001 | 72116000 | 1000 | 1 | 1.31E−06 | −0.8874394 | 13 | 1.3 | NHSL2 | |
| X: 73379001 | X | 73379001 | 73380000 | 1000 | 1 | 9.24E−06 | −0.8777041 | 5 | 0.5 | | |
| X: 91549001 | X | 91549001 | 91550000 | 1000 | 1 | 3.45E−06 | 0.8262897 | 21 | 2.1 | | |
| X: 97607001 | X | 97607001 | 97609000 | 2000 | 1 | 2.72E−06 | −0.7725409 | 24 | 1.2 | DIAPH2; DIAPH2-AS1 | Cytoskeleton |
| X: 118476001 | X | 118476001 | 118479000 | 3000 | 1 | 3.48E−07 | 0.7506308 | 56 | 1.867 | | |
| X: 123148001 | X | 123148001 | 123149000 | 1000 | 1 | 8.23E−06 | 0.9915717 | 5 | 0.5 | | |
| X: 123827001 | X | 123827001 | 123831000 | 4000 | 1 | 3.22E−06 | 0.5590727 | 108 | 2.7 | | |
| X: 125538001 | X | 125538001 | 125539000 | 1000 | 1 | 4.60E−07 | −1.1559361 | 3 | 0.3 | | |
| X: 127483001 | X | 127483001 | 127484000 | 1000 | 1 | 1.80E−06 | −1.0343179 | 7 | 0.7 | | |
| X: 130717001 | X | 130717001 | 130718000 | 1000 | 1 | 1.53E−06 | −0.4949376 | 5 | 0.5 | ENOX2 | Transcription |
| X: 135215001 | X | 135215001 | 135216000 | 1000 | 1 | 9.76E−06 | −0.939075 | 13 | 1.3 | AC234771.2 | |
| X: 141265001 | X | 141265001 | 141266000 | 1000 | 1 | 1.11E−08 | 0.9501535 | 14 | 1.4 | RBMX2P2 | |
| X: 151196001 | X | 151196001 | 151197000 | 1000 | 1 | 7.27E−06 | −1.0056685 | 6 | 0.6 | | |
| X: 155134001 | X | 155134001 | 155135000 | 1000 | 1 | 1.52E−08 | −1.1075893 | 6 | 0.6 | BX293995.1; MTCP1 | |
| X: 155830001 | X | 155830001 | 155832000 | 2000 | 1 | 2.05E−07 | −0.5866864 | 13 | 0.65 | AMD1P2 | |
| Y: 26328001 | Y | 26328001 | 26329000 | 1000 | 1 | 7.16E−06 | 0.7192894 | 36 | 3.6 | PPP1R12BP1 | |

The genomic features of the offspring autism susceptibility DMRs were investigated. The chromosomal locations of the DMRs at p<1e-05 within the human genome are presented in FIG. 1B. The arrowheads (triangles) indicate the individual DMRs, and the black boxes represent a cluster of DMRs. The DMRs are present on all chromosomes. The CpG density of the DMRs is generally less than 10 CpG per 100 bp with 1-3 CpG predominant for the paternal offspring autism susceptibility DMRs, FIG. 1C. The size of the DMRs was predominantly 1-3 kb for the sperm DMRs, FIG. 1D. Additional genomic features are presented in Table 3. The log-fold-change (LFC) in Table 3 demonstrated approximately 60% of the DMRs have an increase in DNA methylation, and the rest a decrease in DNA methylation. Therefore, the majority of the sperm DMRs had low CpG density, termed a CpG desert, and were 1 kb in length with both an increase or decrease in DNA methylation.

The paternal offspring autism susceptibility sperm DMR associated genes and corresponding gene functional categories were determined, as presented in Table 3. The functional categories corresponding to each DMR associated gene are summarized in FIG. 2A. The signaling, transcription, and metabolism functional categories are predominant. This reflects that these gene functional categories have the highest number of genes within them. A comparison of previously identified genes associated with neurodegenerative disease and autism with the DMR associated genes of this study are summarized in FIG. 2B. These autism-associated genes have previously been shown to be regulated or involve genetic mutations within autism patients and the gene symbols, descriptions and associated references are presented in Table 4. The DMR associated genes were also used in a gene pathway or gene set analysis to identify associated pathways. Interestingly, the top pathway or gene set identified was autism and the majority of the subsequent pathways with greater than three genes were all neurodevelopmental or neuro-pathology associated pathways, listed in Table 2. All those gene sets were found to be significant and a list of the specific DMR associated genes are provided in Table 2. Therefore, the DMR associated genes did correlate well with previously identified autism and neurodevelopment associated genes.

TABLE 2
DMR Associated Gene Pathways or Gene Sets

| Pathway or | Total # of | Pathology | | Percent | | |
|---|---|---|---|---|---|---|
| GeneSet Name | Neighbors | Gene Set Pathway | Overlap | Overlap | Overlapping Genes | p-value |
| Protein regulators | 313 | autism | 19 | 6 | RBFOX1, CD5, CCR5, GRIN1, | 9.02E−05 |
| ofautism | | | | | GPHN, SLC7A5, SNTG2, | |
| | | | | | RELN, ARHGAP32, OPRM1, | |
| | | | | | DLGAP2, DIAPH3, CACNA1A, | |
| | | | | | KCNMA1, TSHZ3, SLC25A12, | |
| | | | | | NRXN3, SEMA3F, RORA | |
| Protein regulators | 616 | intellectualdisability | 28 | 4 | SRGAP3, ERBB4, DMD, TG, AHI1, | 3.42E−04 |
| ofintellectual | | | | | ANKRD11, CHL1, ST3GAL5, | |
| disability | | | | | BCKDK, CDK19, DLGAP2, NARS2, | |
| | | | | | PHF21A, KDM2B, CAMTA1, | |
| | | | | | MCPH1, CREBBP, ARHGEF10, | |
| | | | | | TCF4, LIMK1, STIM1, RELN, | |
| | | | | | PRMT7, DPYSL2, SORL1, LRMDA, | |
| | | | | | CDH18, EXOC4 | |
| Protein regulators | 273 | neurodevelopmental | 16 | 5 | RBFOX1, SRGAP3, GRIN1, | 4.70E−04 |
| of | | disorder | | | LIMK1, AHI1, DOCK3, ANKS1B, | |
| neurodevelopmentaldisorder | | | | | ELP4, ANKRD11, RELN, DPYSL2, | |
| | | | | | ST3GAL5, AGAP1, MCPH1, | |
| | | | | | CREBBP, TCF4 | |
| Protein regulators | 538 | psychiatricdisorder | 25 | 4 | GRIN2B, PDE2A, ERBB4, | 5.12E−04 |
| ofpsychiatric | | | | | AHI1, CCKBR, ANKS1B, | |
| disorder | | | | | DLGAP1, CCDC141, OPRM1, | |
| | | | | | PDLIM5, GABBR1, SORCS2, | |
| | | | | | CREBBP, ARHGEF10, TCF4, | |
| | | | | | DNMT1, RBFOX1, GPHN, | |
| | | | | | USP46, RELN, DPYSL2, | |
| | | | | | ADRA2C, IMMP2L, NRXN3, ALK | |
| Protein regulators | 18 | intellectual | 4 | 21 | GRIN2B, GRIN1, DMD, RELN | 5.95E−04 |
| ofintellectual | | impairment | | | | |
| impairment | | | | | | |
| Protein regulators of | 86 | estrogen | 8 | 9 | KDM6B, PPARG, ERBB4, | 6.90E−04 |
| estrogen receptor- | | receptor- | | | PIK3CD, NDRG1, CREBBP, | |
| positive breast cancer | | positive | | | SLC7A5, STIM1 | |
| | | breast cancer | | | | |
| Protein regulators | 147 | behavioral | 10 | 6 | GRIN2B, GRIN1, ERBB4, PARK7, | 1.80E−03 |
| ofbehavioral | | disorder | | | STX1A, RORA, DNMT1, | |
| disorder | | | | | TMEM173, AP2B1, OPRM1 | |
| Protein regulators | 14 | severe | 3 | 20 | PAX2, AIPL1, MERTK | 3.39E−03 |
| ofsevere visual | | visual | | | | |
| impairment | | impairment | | | | |

TABLE 4
DMR associated Gene and Protein Regulators of Autism

| Gene | | Literature References |
|---|---|---|
| Name | Gene Description | PMID |
| SLC25A12 | solute carrier family 25 member 12 | 19913066; 16205742; |
| | | 19360665; 19913066; |
| | | 18180767 |
| NRXN3 | neurexin 3 | 23306218 |
| SEMA3F | semaphorin 3F | 30635860 |
| RORA | RAR related orphan receptor A | 27179922; 26625251; |
| | | 1336 |
| RBFOX1 | RNA binding fox-1 homolog 1 | 18329129; 17503474 |
| CD5 | CD5 molecule | 28979127 |
| CCR5 | C-C motif chemokine receptor 5 | 28986277 |
| | (gene/ | |
| GRIN1 | glutamate ionotropic receptor | 31299220 |
| | NMDA ty | |
| GPHN | gephyrin | 25149987 |
| SLC7A5 | solute carrier family 7 member 5 | 27912058 |
| SNTG2 | syntrophin gamma 2 | 17292328; 17292328 |
| RELN | reelin | 15749247; 15560956; |
| | | 19359144; 25450950; |
| | | 28966264; 26285919; |
| | | 28966264; 15820235; |
| | | 12192627 |
| ARHGAP32 | Rho GTPase activating protein 32 | 30045817 |
| OPRM1 | opioid receptor mu 1 | 21525276 |
| DLGAP2 | DLG associated protein 2 | 28407363 |
| DIAPH3 | diaphanous related formin 3 | 20308993; 20308993 |
| CACNA1A | calcium voltage-gated channel | 28799511; 26566276; |
| | subunit | 26566276; 25735478; |
| | | 26566276 |
| KCNMA1 | potassium calcium-activated | 17236127 |
| | channel s | |
| TSHZ3 | teashirt zinc finger homeobox 3 | 27668656 |

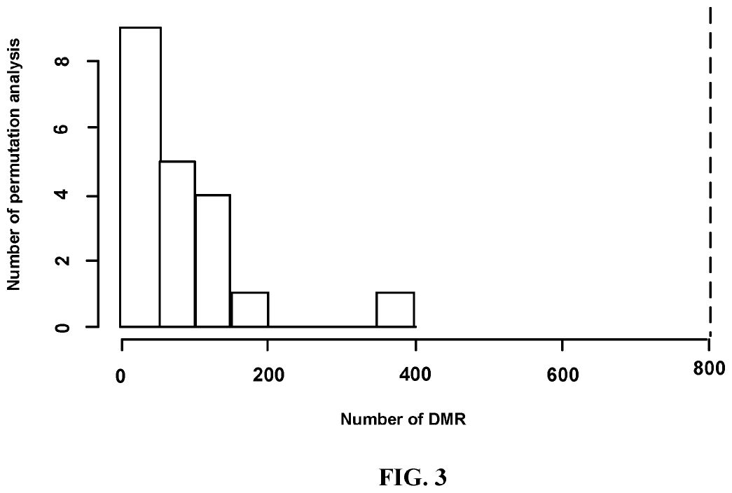

The final analysis examines the statistical significance and validation of the DMRs for the paternal offspring autism susceptibility. Initially, a permutation analysis was performed on the DMRs to demonstrate the DMRs were not due to background variation in the data and randomly generated. The permutation analysis shows the number of DMRs generated from the control versus autism case comparison was significantly greater than the DMRs generated from random subsets within the analysis, in FIG. 3. The dashed line to the right indicates the comparison DMRs versus the low numbers from the random subset comparison. Another analysis involved a cross validation of the DMRs and demonstrated approximately 80% accuracy in the confirmation of the DMRs to assess autism susceptibility. A principal component analysis (PCA) of the control male sperm without an autistic child versus the male sperm with an autistic child is presented in FIG. 2C. A clear separation of the DMR principal components is seen between the groups. This demonstrates a distinction between the DMR principal components. The current disclosure provides that epigenetic biomarkers exist and may be used to diagnose that a father may have an autistic child.

The frequency of autism in the population has dramatically increased over tenfold the past several decades. This increase appears to be due in part to increased diagnosis efficiency from 1975 to the early 2000's, as well as greater public awareness of the disease. The more recent increase in the last couple of decades suggests environmental factors and exposures also have a role in autism prevalence. Although many suggestions have been made on specific toxicants and factors being involved, more extensive analysis and better understanding of autism etiology can be needed to understand this increase in autism frequency. An example is the suggestion assisted reproduction and in vitro fertilization are involved but follow up studies demonstrated no risk of ASD in children born after assisted reproduction. One factor that has been correlated with autism is paternal age and sperm DNA methylation alterations. Previous studies have shown a hypermethylation of sperm DNA can be associated with male infertility, abnormal sperm parameters, and increasing age. Therefore, the majority of DMR involve an increase in DNA methylation when associated with infertility or age. The current study demonstrated 60% of the DMRs have an increase in DNA methylation and 40% of DMRs decreases in DNA methylation, as listed in Table 3. Therefore, a mixture of an increase and decrease in methylation can be observed, which can be distinct from the sperm hypermethylation observed in male infertility and aging. Since all the paternal patients were fertile and generally younger ages, the current study observations appear to be distinct from infertility and aging DNA hypermethylation. Therefore, the current disclosure was designed to identify a sperm epigenetic biomarker to assess a father's ability to transmit autism susceptibility to his offspring.

Altered germline epigenetics has been shown to impact offspring health later in life, and if permanently programmed, to promote the epigenetic transgenerational inheritance of disease and pathology to subsequent generations. Since sperm or egg epigenetics can impact the zygote epigenetics and transcription following fertilization, as well as the subsequent stem cell population in the early embryo epigenetics and transcription, all subsequently derived somatic cells also have the potential to have an altered cell type specific epigenomes and transcriptomes later in development. This molecular alteration has been shown to be associated with adult somatic cell epigenetics, transcriptomes, and associated diseases. The ability of an ancestral or early life exposure to impact the germline epigenetics to then subsequently impact the offspring epigenetics and susceptibility to develop pathology and disease has been established, and may be anticipated to be a component of autism etiology as well.

The application of a sperm molecular diagnostic can be used in an assisted reproduction setting. Routine semen analysis and genetic testing can be used in most in vitro fertilization clinical settings. Although epigenetic analysis is not as routine, the proposal for such analysis may be made. The analysis of male infertility using sperm DNA methylation alterations has been developed. Epigenetic alterations (DNA methylation) in sperm have been shown to associate in fathers of families with autistic children. That study used a targeted array-based approach that focused on high density CpG islands that constitute approximately 1% of the genome, but does demonstrate such an analysis can be feasible. The current disclosure provides a genome-wide approach to identify altered DNA methylation for paternal sperm and offspring autism susceptibility.

Although genetics may be involved in autism etiology, genome-wide association studies (GWAS) have demonstrated generally less than 1% of the patients with a specific disease, such as neurodegenerative disease, have a correlated genetic mutation. ASD can be similar with only a few percent correlation with associated genetic mutations. An additional molecular mechanism to consider for ASD disease etiology involves epigenetics. The current study uses a more genome-wide approach to investigate sperm DNA methylation in fathers with or without autistic children. A procedure to assess DNA methylation alterations in low density CpG regions that constitute over 95% of the human genome was used in comparison to the high density CpG procedures previously used. A highly significant and reproducible signature of differential DNA methylation regions (DMRs) was identified comparing the sperm from fathers with or without autistic children. The genomic features of the DMRs were identified and demonstrated generally 1 kb lengths and low density CpG regions. The DMR associated genes were identified, and a number of previously identified autism linked genes were present (FIG. 2B, Tables 2 and 4). In regard to the autism sperm DMR biomarkers, a strong separation in a principal component analysis (PCA) was observed. In addition to this validation, the permutation and cross validation analysis demonstrated the robustness and sensitivity of the analysis. The observations demonstrate the paternal sperm epigenetic analysis can be effective at identifying offspring susceptibility for autism. The current disclosure provides a diagnostic for autism susceptibility may be developed.

Although a reproducible epigenetic signature was identified for paternal transmission of susceptibility of autism children was identified and statistically significant, a limitation of the current study may be the low number of samples used for the analysis. Expanded clinical trials are required with increase numbers, greater ethnic diversity, and more thorough assessment of the impacts of paternal age. The impacts of these variables need to be elucidated to improve and expand the accuracy of the analysis. The expanded clinical trial with greater numbers and diverse subpopulations may be important to develop a diagnostic. However, the current study does provide a diagnostic may be developed.

Applications of the paternal offspring autism susceptibility biomarker / diagnostic may potentially improve the health care for ASD patients. This would allow IVF patients to assess risk and determine management procedures. Importantly, this would allow clinicians to plan the offspring's clinical management options more efficiently. Potential preventative treatments could be considered to reduce the severity of the autism spectrum disorder. The availability of the assay could also be used in a research setting to facilitate the identification of environmental factors potentially involved in the ASD etiology. Therefore, potential therapeutic and preventative options not previously considered could be taken.

The current disclosure identified a genome-wide signature of DNA methylation sites that are associated with the paternal transmission of offspring autism susceptibility. The current disclosure provides the proof of concept for the assay and biomarkers. Therefore, the identification of offspring susceptibility can be assessed, allowing better clinical management of ASD. The potential for therapy options may be expanded to improve health care for ASD. Such epigenetic biomarkers are anticipated to exist for many disease and pathology conditions, which may facilitate the future preventative medicine strategies for health care.

### Methods

### Clinical Sample Collection

A single center (IVIRMA Valencia, Spain) prospective and open clinical study was performed. The participant approval was obtained prior to the clinical sample collection. The study protocols were approved by the Institutional Review Board 1311-VLC-136-FC. The semen was analyzed as described in the Supplemental Methods. Samples were immersed in liquid nitrogen and then stored at −20 C. prior to analysis.

### Epigenetic Analysis, Statistics and Bioinformatics

Sperm DNA was isolated as previously described 15. Methylated DNA immunoprecipitation (MeDIP), followed by next generation sequencing (MeDIP-Seq) was performed. MeDIP-Seq, sequencing libraries, next generation sequencing, and bioinformatics analysis were performed as described, and are found in the Supplemental Methods. The statistical analysis and validation protocols were performed as previously described, and are found in the Supplemental Methods. All molecular data has been deposited into the public database at NCBI (GEO # pending), and R code computational tools are available at GitHub (https://github.com/skinnerlab/MeDIP-seq) and www.skinner.wsu.edu.

### Example 2

Further studies are conducted to increase the sensitivity of the prediction model of DNA methylation signature in father's sperm that is predictive of ASD in context of more study and control participants. The commercialization of a validated DNA methylation signature to predict the susceptibility of a father having offspring with ASD would be instrumental in increasing the rates of early diagnosis and therapeutic interventions. With this test, expecting parents at higher risk or concerned about a potential ASD diagnosis for their child can better understand their potential of having a child with ASD and drive more vigilant developmental assessments and diagnosis.

### Example 3

More studies are conducted to transition the examination of these biomarkers to a more commercial platform. The current discovery research and algorithmic model was developed using methylation immunoprecipitation (MeDIP) technology. A scalable and more cost-effective platform to conduct the ASD prediction test from the father's sperm using targeted sequencing technology is implemented. An at-home semen collection kit provided to expecting fathers by a couple's obstetrician. The fathers may collect a semen sample and ship the sample directly to a lab for processing and analysis. Results may be provided to the ordering obstetrician, similar to the standard data-flow for paternal carrier testing. Additional research, may focus on 1) integrating the refined and scalable test into a fully regulated, CAP accredited and CLIA certified, workflow and 2) developing an appropriate physician and patient facing report. A report of this type may require significant input from both patients and physicians due to the sensitivities of an ASD prediction. Subsequent research may interrogate the existence of any of the paternal methylation patterns, or other unique methylation patterns, in young children diagnosed with ASD. This subsequent research may hopefully lead to commercialization of a newborn screening diagnostic for ASD.

### Patient Cohort Distribution

About 60 fathers between the ages of 30-45 who have a single offspring with the diagnosis of ASD, level 1, 2, or 3, no known family history of ASD, and no identified genetic diagnosis of ASD are participating in the study. ASD diagnosis is required from a comprehensive diagnostic evaluation following the criteria and standardization provided by the American Psychiatric Association's Diagnostic and Statistical Manual, Fifth Edition (DSM-5). Additionally, for this study, diagnosis is required by a qualified Pediatric Psychologist, Pediatric Physiatrist, Pediatric neurologist, or Developmental Pediatrician. Currently participants with a known family history of ASD, or an identified genetic diagnosis may be excluded to remove the variable of genetic inheritance into this study. In the cases of familial inheritance or germline genetic mutations, it is likely that the DNA code plays a more significant role in ASD risk than DNA methylation. In addition to the diagnosis, all co-occurring conditions associated with the ASD individuals as well as basic anthropometric measurements (i.e. height, weight, sex, age etc.) of father and offspring may be collected.

Further semen samples are collected from the subjects and processed. Processing includes:
1. Counting of sperm under a microscope to determine concentration, following the World Health Organization guidelines for sperm counting
2. Isolation of sperm cells from somatic cells
3. DNA extraction from a pure sperm cell population
4. DNA fragmentation into 200-400 base pairs
5. Methylated DNA immunoprecipitation (MeDIP) to capture methylated genomic regions
6. DNA purification
7. High-Throughput DNA Illumina DNA sequencing

### Data Analysis

Bioinformatic analysis of sequencing results may first be done blinded to cohort type. Reads from each sample may be mapped back to HG19 human genome. Utilizing the R programming language, the differential sequencing coverage as well as the relative DNA methylation coverage between samples are calculated. Samples are re-identified and analyzed for consistent and reproducible patterns that are predictive of offspring with an ASD diagnosis. A process for high-fidelity analysis that includes is utilized:
1. Filtering out locations with inadequate coverage.
2. Aligning all reads to the human genome to identify methylated regions of DNA.
3. Quantifying how many sequencing reads fall into each genomic window of differential methylation.
4. Comparing the methylated regions between samples.
5. Cohort Analysis identifying any statistical difference in the DNA methylation patterns between samples.

The model using sequencing data is retained. The model for application on targeted sequencing data is updated and tuned to accommodate nuances in the data that arise from the data being generated on a different platform. The model is adjusted to compensate for any differences that are present between MeDIP and sequencing data. Further, the model is continuously updated using larger numbers of samples when more samples are collected over time.

### Sample Preparation

All validation steps required under the regulatory guidance of CAP/CLIA are followed for sample preparation, sequencing, and analysis. Effort may be focused on the amplification of previously identified 223 genomic regions (ranging in size from 500 -2000 kb).

The samples are thawed and subjected to somatic cell lysis to ensure the elimination of any potentially contaminating non-sperm cells followed by DNA extraction. For somatic cell lysis, the thawed samples are washed in 14 ml of PBS followed by two washes in 14 ml of distilled water. The sample are then centrifuged, and the resulting pellet incubated for a minimum of 60 minutes a 4° C. in 14 ml of a somatic cell lysis buffer (0.1% SDS, 0.5% Triton X-100 in DEPC H2O). Following somatic cell lysis, sperm DNA is isolated using a sperm-specific modification to a column-based extraction protocol using the DNeasy DNA isolation kit (Qiagen, Valencia CA). Extracted sperm DNA is bisulfite converted with EZ-96 DNA Methylation-Gold kit (Zymo Research, Irvine CA).

Targeted amplification and sequencing of the differentially methylated genomic regions are completed using ThermoFisher's Ion Ampliseq technology which includes QuantStudio real-time PCR (amplification) and the Ion Torrent S5 (next generation sequencing). Due to the bisulfite converted state of the sperm DNA, primer design requires a manual design service provided by ThermoFisher. As bisulfite conversion changes unmethylated cytosines to uracil, primers need to be designed to bind to regions that do not contain base-pairs that may be converted to uracil. Additionally, bisulfite converted DNA requires three-times the number of primers compared to native DNA in order to effectively amplify the genomic regions. The proper primer design can be important to get the depth of amplification needed for analysis. Additionally, due to the subtle changes of methylation, we require 1000× depth of coverage for each site.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. A method, comprising:
obtaining a sperm sample from a human male subject;
isolating deoxyribonucleic acid (DNA) from the sample;
determining a methylation level of a differential DNA methylation region (DMR) comprised in the isolated DNA; and
comparing the methylation level of the DMR to a reference level of a corresponding reference DMR;
wherein:
the comparing comprises comparing employing a computer comprising a computer processor and computer readable memory comprising computer readable instructions contained thereon;
the determining comprises a methylated DNA immunoprecipitation (MeDIP), a sequencing, a bisulfite treatment, a bisulfite conversion, a deamination of an unmethylated cytosine base, employing an array, or any combination of these;
wherein about 90 to about 1000 distinct DMRs are detected and compared; and
the about 90 to about 1000 distinct DMRs are selected from the DMRs in Table 3.

2. The method of claim 1, wherein about 200 to about 1000 distinct DMRs, about 300 to about 1000 distinct DMRs, about 400 to about 1000 distinct DMRs, about 500 to about 1000 distinct DMRs, about 600 to about 1000 distinct DMRs, about 700 to about 1000 distinct DMRs, about 800 to about 1000, or about 900 to about 1000 distinct DMRs are detected.

3. The method of claim 1, comprising sequencing, and wherein the sequencing comprises sequencing by synthesis, ion semiconductor sequencing, single molecule real time sequencing, nanopore sequencing, next-generation sequencing, or any combination thereof.

4. The method of claim 1, wherein
the detected DMRs comprise DMRs from at least about: 1, 2, 3, 4, 5, 6, 7, 8, 9, 10, 11, 12, 13, 14, 15, 16, 17, 18 19, 20, 21, 22, or 23, chromosomes; or
the detected DMRs are DMRs are from at least about: 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.

5. The method of claim 1, wherein the sperm sample is obtained from a human male subject at least about: 1 day, 2, days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 51 years, 52 years, 53 years, 54 years, 55 years, 56 years, 57 years, 58 years, 59 years, 60 years, 61 years, 62 years, 63 years, 64 years, 65 years, 66 years, 67 years, 68 years, 69 years, 70 years, 71 years, 72 years, 73 years, 74 years, 75 years, 76 years, 77 years, 78 years, 79 years, 80 years, 81 years, 82 years, 83 years, 84 years, 85 years, 86 years, 87 years, 88 years, 89 years, 90 years, 91 years, 92 years, 93 years, 94 years, 95 years, 96 years, 97 years, 98 years, 99 years, or 100 years of age.

6. The method of claim 1, wherein the sperm sample is obtained from a human male subject of an age ranging from about 15 years to about 80 years of age.

7. The method of claim 1, wherein the DMRs that are determined and compared, individually, range from about 100 to about 17000 adjacent nucleotides.

8. The method of claim 1, wherein at least a plurality of the DMRs that are determined and compared comprise a CpG density of less than about 10 CpG per 100 nucleotides.

9. The method of claim 8, wherein at least a plurality of the DMRs that are determined and compared comprise a CpG density of less than about 3 CpG per 100 nucleotides.

10. The method of claim 2, wherein at least about: 30, 40, 50, 60, or 70 percent of the DMRs that are determined and compared are hypermethylated when compared, individually, to individual reference methylation levels of corresponding individual reference DMRs.

11. (canceled)

12. The method of claim 1, wherein the method further comprises, determining with a computer, a risk of an offspring of the human male subject having a disease or condition.

13. The method of claim 1, wherein the method further comprises, determining with a computer, a severity of autism spectrum disorder of an offspring of the human male subject.

14. The method of claim 1, wherein the method further comprises, determining with a computer, a severity of autism spectrum disorder of the human male subject.

15. The method of claim 12, wherein the disease or condition comprises autism or autism spectrum disorder.

16. The method of claim 12, wherein the disease or condition is selected from the group consisting of disease related to autism or neurodegenerative disease, such as Asperger's syndrome.

17. The method of claim 1, further comprising performing a further analysis using a computer

18. The method of claim 17, wherein the further analysis comprises a principle component analysis (PCA), a dendrogram analysis, a machine learning analysis, or any combination thereof.

19. The method of claim 17, wherein the further analysis generates data points, and wherein the data points in the further analysis are grouped into two spatially distinct categories—a first category which indicates the subject or an offspring of the subject is at increased risk of having a disease or condition and second category which indicates the subject or the offspring of the subject is not at increased risk of having the disease or condition.

20. A method, comprising:
obtaining a sperm sample from a human male subject;
isolating deoxyribonucleic acid (DNA) from the sample;
determining a methylation level of a differential DNA methylation region (DMR) comprised in the isolated DNA; and
comparing the methylation level of the DMR to a reference level of a corresponding reference DMR;
wherein:
the comparing comprises comparing employing a computer comprising a computer processor and computer readable memory comprising computer readable instructions contained thereon;
the determining comprises a methylated DNA immunoprecipitation (MeDIP), a sequencing, a bisulfite treatment, a bisulfite conversion, a deamination of an unmethylated cytosine base, employing an array, or any combination of these; and
wherein a number of determined DMRs are sufficient to determine, from a process comprising the comparing and employing a computer, whether the human male subject, or an offspring of the human male subject, has or is at increased risk of having autism or autism spectrum disorder, or determine a severity of autism spectrum disorder.

21. The method of claim 20, wherein about 90 to about 1000 distinct DMRs are determined and compared.

22. (canceled)

23. The method of claim 20, further comprising treating the human male subject or an offspring thereof.

24. The method of claim 23, comprising treating the offspring of the human male subject, wherein the offspring comprises at least one cell, treating the human male subject, or treating a sperm cell of the human male subject or a male offspring of the human male subject.

25. The method of claim 23, comprising treating the offspring of the human male subject, wherein the offspring is less than about 2 years old.

26. The method of claim 23, wherein the treating comprises administering an applied behavior analysis, a cognitive behavior therapy, an educational therapy, a joint attention therapy, a nutritional therapy, an occupational therapy, a physical therapy, a social skills training, a speech language therapy, an antipsychotic drug or a salt thereof, risperidone or a salt thereof, aripiprazole or a salt thereof, a selective serotonin re-uptake inhibitor or a salt thereof, citalopram or a salt thereof, escitalopram or a salt thereof, fluoxetine or a salt thereof, fluvoxamine or a salt thereof, paroxetine or a salt thereof, sertraline or a salt thereof, dapoxetine or a salt thereof, indalpine or a salt thereof, zimelidine or a salt thereof, alaproclate or a salt thereof, centpropazine or a salt thereof, femoxetine or a salt thereof, omiloxetine or a salt thereof, panuramine or a salt thereof, seproxetine or a salt thereof, venlafaxine or a salt thereof, clomipramine or a salt thereof, methylphenidate or a salt thereof, mixed amphetamine salts, a psychoactive medication or a salt thereof, a stimulant or a salt thereof, a valproic acid or a salt thereof, phenytoin or a salt thereof, clonazepam or a salt thereof, carbamazepine or a salt thereof, a social skills therapy, speech therapy, supplementing a vitamin or a salt thereof, a mineral or a salt thereof, or both, a restricted diet, a risperidone or a salt thereof, or any combination thereof.

27. The method of claim 24, wherein the treating comprises administering a therapeutically effective amount of a pharmaceutical formulation to the subject.

28. The method of claim 27, wherein the pharmaceutical formulation comprises a pharmaceutically acceptable: excipient, diluent, or carrier.

29. The method of claim 27, wherein the pharmaceutical formulation is in unit dose form.

30. The method of claim 27, wherein the pharmaceutical formulation is administered orally, intranasally, by inhalation, sublingually, by injection, by a transdermally, intravenously, subcutaneously, intramuscularly, in an eye, in an ear, in a rectum, intrathecally, or any combination thereof.

31. The method of claim 27, wherein the pharmaceutical formulation is administered in an amount ranging from about 0.0001 to about 100,000 mg of pharmaceutical formulation per kg of subject body weight or offspring of subject body weight.

32. The method of claim 1, further comprising transmitting data, a result, or both, via an electronic communication medium.

33. A kit comprising at least about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence present in Table 3; and a container.

34. A kit comprising at least about: 1, 2, 3, 4, 5, 6, 7, 8, 9 10, 11, 12, 13 14, 14, 16, 17, 18, 19, 20, 30, 40, 50, 60,70, 80, 90, 100, 200, 300, 400, 500, 600, 700, 800, 900, or 1000 distinct probes, each distinct probe complementary to a distinct DMR sequence present in Table 3; and a container.

35. The kit of claim 34, wherein the distinct probes further comprises at least one of fluorophore, a chromophore, a barcode, or any combination thereof.

36. The kit of claim 35, wherein each probe comprises a unique:
fluorophore, a chromophore, barcode, or any combination thereof.

37. The kit of claim 33, wherein the distinct primers or pairs of primers each further comprise a unique barcode.

38. The kit of claim 33, wherein the probes or the primers are not bound to an array or a microarray.

39. The kit of claim 33, wherein the probes or the primers are bound to an array or a microarray.

40. The kit of claim 33, wherein the probes, the primers, or both comprise DNA.

41. A method, comprising:
obtaining a sperm sample from a human male subject;
isolating deoxyribonucleic acid (DNA) from the sample;
fragmenting the DNA;
isolating fragmented methylated DNA;
determining a methylation level of a differential DNA methylation region (DMR) comprised in the isolated fragmented methylated DNA; and
comparing the methylation level of the DMR to a reference level of a corresponding reference DMR;
wherein:
the comparing comprises comparing employing a computer comprising a computer processor and computer readable memory comprising computer readable instructions contained thereon;
the determining comprises: amplification of the isolated fragmented methylated DNA, sequencing the isolated fragmented methylated DNA, an amplicon thereof, or both, employing an array, or any combination of these;
wherein about 90 to about 1000 distinct DMRs are detected and compared; and
the about 90 to about 1000 distinct DMRs are selected from the DMRs in Table 3.

42. The method of claim 41, wherein the isolating the fragmented methylated DNA comprises methylated DNA immunoprecipitation (MeDIP).

43. A method, comprising:
obtaining a sperm sample from a human male subject;
isolating deoxyribonucleic acid (DNA) from the sample;
fragmenting the DNA;
isolating fragmented methylated DNA;
determining a methylation level of a differential DNA methylation region (DMR) comprised in the isolated fragmented methylated DNA; and
comparing the methylation level of the DMR to a reference level of a corresponding reference DMR;
wherein:
the comparing comprises comparing employing a computer comprising a computer processor and computer readable memory comprising computer readable instructions contained thereon;
the determining comprises: amplification of the isolated fragmented methylated DNA, sequencing the isolated fragmented methylated DNA, an amplicon thereof, or both, employing an array, or any combination of these; and
wherein a number of determined DMRs are sufficient to determine, from a process comprising the comparing and employing a computer, whether the human male subject, or an offspring of the human male subject, has or is at increased risk of having autism or autism spectrum disorder, or determine a severity of autism spectrum disorder.

44. The method of claim 43, wherein the isolating the fragmented methylated DNA comprises methylated DNA immunoprecipitation (MeDIP).
